(19) United States
(12) Patent Application Publication
(10) Pub. No.: US 2024/0415828 A1
(43) Pub. Date: Dec. 19, 2024

(54) TLR7 AGONIST AND COMBINATIONS FOR CANCER TREATMENT

(71) Applicant: BIONTECH SE, Mainz (DE)

(72) Inventors: Ugur SAHIN, Mainz (DE); Hariz Iskandar Bin HASSAN, Mainz (DE); Stefan STROBL, Mainz (DE); Roman RÖSEMANN, Mainz (DE)

(21) Appl. No.: 18/704,537

(22) PCT Filed: Nov. 9, 2022
(86) PCT No.: PCT/EP2022/081274

§ 371 Date: Apr. 25, 2024

(30) Foreign Application Priority Data
Nov. 9, 2021 (WO) PCT/EP2021/081164

(51) Int. Cl. A61K 31/4745; A61K 9/00; A61K 31/555; A61K 31/7048; A61K 39/00; A61P 35/00; C07D 471/04; C07K 16/28

(52) U.S. Cl. CPC A61K 31/4745; A61K 9/0019; A61K 31/555; A61K 31/7048; A61P 35/00; C07D 471/04; C07K 16/2827; A61K 2039/505

(57) ABSTRACT

The present disclosure relates to the specific TLR7 agonist BNT411, such as compositions and kits comprising BNT411, and the use of BNT411 as immunotherapeutic agent in monotherapy and combination therapy to reduce or prevent progression of a tumor, in particular a solid tumor, or treating cancer, in particular a solid cancer.

### TECHNICAL FIELD

The present disclosure relates to the specific TLR7 agonist BNT411, such as compositions and kits comprising BNT411, and the use of BNT411 as immunotherapeutic agent in monotherapy and combination therapy to reduce or prevent progression of a tumor, in particular a solid tumor, or treating cancer, in particular a solid cancer.

### BACKGROUND

Cancer immunotherapy offers the option of long-term disease control by activating the patient's own immune system and inducing T-cell memory. A promising approach involves Toll-like receptors (TLRs), a group of receptors belonging to the innate immune system. TLR7 in particular is known to contribute to potent anti-tumor responses. Stimulation of TLR7 triggers intracellular pathways that culminate in the activation of the adaptive and innate immune system, characterized by the induction of type 1 interferons (IFNs), proinflammatory cytokines, chemokines and in the upregulation of costimulatory molecules. These TLR7-mediated effects can contribute to tip the balance towards robust cancer immunity by enhancing pre-existing immunity via pleiotropic mechanisms and by inducing de novo anti-tumor immune responses.

Various synthetic TLR7 agonists, including the imidazoquinolines imiquimod (R837), resiquimod (R848) and 852A, are being studied as immunotherapy drugs.

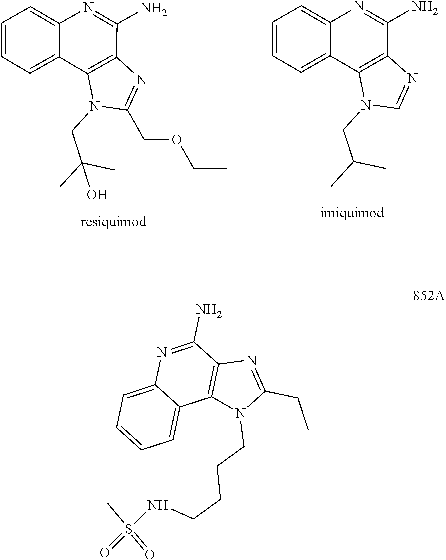 [SMILES: CCOCc1nc2c(N)nc3ccccc3c2n1CC(C)(C)O ; CC(C)Cn1cnc2c(N)nc3ccccc3c21 ; CCc1nc2c(N)nc3ccccc3c2n1CCCCNS(C)(=O)=O]

So far only imiquimod is approved for human use, and only topical cream for basal cell carcinoma. Clinical application of these compounds is severely limited partly due to dose limiting adverse reactions upon systemic administration. This safety concern is thought to be linked to TLR8 coactivation resulting in induction of a broader spectrum of proinflammatory cytokines.

Thus, there is a considerable need for improved therapies, in particular therapies having less adverse reactions upon systemic administration, to prevent progression of a tumor or treating cancer.

Furthermore, there is a strong unmet medical need to develop new efficacious therapies for patients with advanced malignancies whose disease no longer responds to standard therapy, but also for patients where current standard of care results in only marginal long-term disease-free survival (e.g., extensive-stage small cell lung (ES-SCLC)). Refinements in conventional therapies such as chemotherapy, radiotherapy, surgery, and targeted therapies and recent advances in immunotherapies have improved outcomes in patients with advanced cancers. However, many immunotherapy treatments have demonstrated efficacy in only a selected group of cancers (Ventola, Cancer Immunotherapy, Part 3: Challenges and Future Trends. 2017; PT 42 (8): 514-21; Yang, J Clin Invest. 2015; 125 (9): 3335-7).

SCLC accounts for approximately 15% of bronchogenic carcinomas. At the time of diagnosis, approximately 30% of patients with SCLC will have tumors confined to the ipsilateral hemithorax and thus limited-stage small cell lung cancer (LS-SCLC) (Murray et al., J. Clin Oncol. 1993; 11 (2): 336-44). Patients with tumors beyond the ipsilateral hemithorax, including malignant pleural or pericardial effusion or hematogenous metastases have ES-SCLC.

Regardless of stage, the current prognosis for patients with SCLC is unsatisfactory despite improvements in diagnosis and therapy made during the past 25 years. Without treatment, SCLC has the most aggressive clinical course of any type of pulmonary tumor, with median survival from diagnosis of only 2 to 4 months. In patients with ES-SCLC, median survival of 6 to 12 months is reported with currently available therapy, but long-term disease-free survival is rare.

Currently, chemotherapy has remained the mainstay of treatment for ES-SCLC. It is commonly given as a two-drug combination of platinum and etoposide in doses associated with at least moderate toxic effects (as in limited-stage small cell lung cancer (LS-SCLC)) (Okamoto et al., Br J Cancer. 2007; 97 (2): 162-9). Cisplatin is associated with significant toxic effects and requires fluid hydration, which can be problematic in patients with cardiovascular disease. Carboplatin is active in SCLC, is dosed according to renal function, and is associated with less non-hematological toxic effects. Doses and schedules used in current programs yield overall response rates of 50% to 80% and complete response rates of 0% to 30% (Pujol et al., Br J Cancer. 2000; 83 (1): 8-15; Roth et al., J Clin Oncol. 1992; 10 (2): 282-91). Radiation therapy to sites of metastatic disease especially brain, epidural, and bone metastases, is a standard treatment option since chemotherapy is unlikely to palliate. Furthermore, ES-SCLC patients treated with chemotherapy who have achieved a response are considered for thoracic radiation therapy. Recently, atezolizumab in combination with chemotherapy has been approved as first-line (1L) treatment. This combination has proven effective but the outcomes are still unsatisfactory compared to other malignant diseases (i.e., 2-month overall survival benefit and 0.9-month progression free survival benefit; NCT02763579. A Study of Carboplatin Plus Etoposide with or Without Atezolizumab in Participants with Untreated Extensive-Stage (ES) Small Cell Lung Cancer (SCLC) (IMpower133)). Almost all patients finally succumb to the diagnosis upon relapse; median survival of 6 to 12 months is reported with currently available therapy, but long-term disease-free survival is rare. Therefore, there is a high unmet medical need to develop new therapies in ES-SCLC, especially an innovative combination therapy to improve outcomes.

### SUMMARY

The present inventors have developed BNT411 which is a highly potent TLR7 agonist of the imidazoquinoline family and has the following formula:

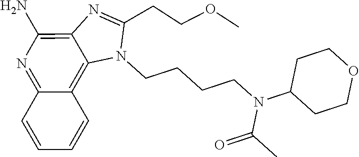 [SMILES: COCCc1nc2c(N)nc3ccccc3c2n1CCCCN(C(C)=O)C1CCOCC1]

BNT411 is a selective TLR7 agonist and showed in vitro, in comparison to the most active imidazoquinoline comparator resiquimod, primarily IFNα release. Moreover, the present inventors surprisingly found that upon BNT411 exposure, a higher amount of IFNα was induced at concentrations much lower than the comparative imidazoquinolines imiquimod, resiquimod and 852A; cf., e.g., FIG. 1. For inducing secretion of pyrogenic proinflammatory cytokines (e.g., TNFα) up to two orders of magnitude higher concentrations of BNT411 are required; cf., e.g., FIG. 7. Taken together, this stronger activity and greater selectivity indicates a higher potency and broader therapeutic window for systemically administered BNT411. In vitro and in vivo studies with BNT411 in mouse models demonstrate release of high levels of therapeutically relevant IFNα; cf., e.g., FIG. 2. In addition, the present disclosure demonstrates that in mouse tumor models BNT411 exhibits anti-tumor activity in monotherapy as well as in combination with standard therapies (such as in combination with a platinum-based chemotherapeutic agent or a checkpoint inhibitor); cf., e.g., FIGS. 4 to 6. Moreover, the present inventors show for the first time that BNT411 (i) has an acceptable safety profile at several doses tested in human patients; (ii) induces a substantial type-I IFN-dominated cytokine response in human patients; and (iii) does not lead to a substantial increase of pyrogenic proinflammatory cytokines such as tumor necrosis factor alpha (TNFα) at least at some therapeutically relevant doses; cf., e.g., Examples 6 and 7 as well as FIG. 8 of the present application.

Thus, in a first aspect, the present disclosure provides a compound having the following formula

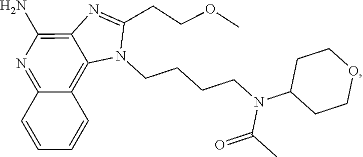 [SMILES: COCCc1nc2c(N)nc3ccccc3c2n1CCCCN(C(C)=O)C1CCOCC1]

or a pharmaceutically acceptable solvate or salt thereof (collectively also referred to as “BNT411” herein unless otherwise stated or contradicted by the context herein), for use in a method for reducing or preventing progression of a tumor or treating cancer in a subject, said method comprising administering to said subject BNT411 in a suitable amount. In some embodiments of the first aspect, the suitable amount of BNT411 is a therapeutically effective and safe amount. In some embodiments, the suitable amount of BNT411 is about 0.1 μg/kg to about 50 μg/kg body weight or about 8 μg to about 4000 μg in total; and/or about 0.23×10⁻⁹ mol/kg body weight to about 114×10⁻⁹ mol/kg body weight or about 18×10⁻⁹ mol to about 9100×10⁻⁹ mol in total. In some embodiments, the suitable amount of BNT411 is about 0.5 μg/kg to about 20 μg/kg body weight or about 40 μg to about 1600 μg in total; and/or about 1.14×10⁻⁹ mol/kg body weight to about 45.5×10⁻⁹ mol/kg body weight or about 91×10⁻⁹ mol to about 3640×10⁻⁹ mol in total. In some embodiments, the suitable amount of BNT411 is about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total. In some embodiments, the suitable amount of BNT411 is about 2.4 μg/kg to about 9.6 μg/kg body weight (such as about 4.8 μg/kg to about 9.6 μg/kg body weight) or about 192 μg to about 768 μg (such as about 384 μg to about 768 μg) in total; and/or about 5.46×10⁻⁹ mol/kg body weight to about 21.8×10⁻⁹ mol/kg body weight (such as about 10.9×10⁻⁹ mol/kg body weight to about 21.8×10⁻⁹ mol/kg body weight) or about 437×10⁻⁹ mol to about 1747×10⁻⁹ mol (such as about 873×10⁻⁹ mol to about 1747×10⁻⁹ mol) in total. In some embodiments, the suitable amount of BNT411 is about 4.8 μg/kg body weight or about 384 μg in total; and/or about 10.9×10⁻⁹ mol/kg body weight or about 874×10⁻⁹ mol in total.

In a second aspect, the present disclosure provides a composition comprising BNT411, i.e., a compound having the following formula

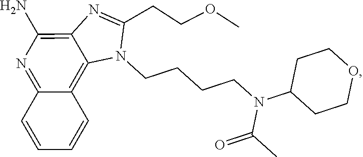 [SMILES: COCCc1nc2c(N)nc3ccccc3c2n1CCCCN(C(C)=O)C1CCOCC1]

or a pharmaceutically acceptable solvate or salt thereof, wherein the amount of BNT411 in the composition is between about 8 μg to 4000 μg or about 18×10⁻⁹ mol to about 9100×10⁻⁹ mol. In some embodiments of the second aspect, the composition comprises from about 40 μg to about 1600 μg or about 91×10⁻⁹ mol to about 3640×10⁻⁹ mol of BNT411. In some embodiments of the second aspect, the composition comprises from about 160 μg to about 800 μg or about 364×10⁻⁹ mol to about 1820×10⁻⁹ mol of BNT411. In some embodiments of the second aspect, the composition comprises from about 192 μg to about 768 μg (such as from about 384 μg to about 768 μg) or about 437×10⁻⁹ mol to about 1747×10⁻⁹ mol (such as about 873×10⁻⁹ mol to about 1747×10⁻⁹ mol) of BNT411. In some embodiments of the second aspect, the composition comprises about 384 μg or about 874×10⁻⁹ mol of BNT411.

In a third aspect, the present disclosure provides a composition of the second aspect for use in a method for reducing or preventing progression of a tumor or treating cancer in a subject. In some embodiments of the third aspect, the composition is administered to the subject in an amount so as to provide about 0.1 μg/kg to about 50 μg/kg body weight of BNT411 or about 8 μg to about 4000 μg of BNT411 in total; and/or about 0.23×10⁻⁹ mol/kg body weight to about 114×10⁻⁹ mol/kg body weight of BNT411 or about 18×10⁻⁹ mol to about 9100×10⁻⁹ mol of BNT411 in total. In some embodiments, the composition is administered to the subject in an amount so as to provide about 0.5 μg/kg to about 20 μg/kg body weight of BNT411 or about 40 μg to about 1600 μg of BNT411 in total; and/or about 1.14×10⁻⁹ mol/kg body weight to about 45.5×10⁻⁹ mol/kg body weight of BNT411 or about 91×10⁻⁹ mol to about 3640×10⁻⁹ mol of BNT411 in total. In some embodiments, the composition is administered to the subject in an amount so as to provide about 2 μg/kg to about 10 μg/kg body weight of BNT411 or about 160 μg to about 800 μg of BNT411 in total; and/or about 4.55×10⁻⁹ mol/kg body weight to about 22.8×10⁻⁹ mol/kg body weight of BNT411 or about 364×10⁻⁹ mol to about 1820×10⁻⁹ mol of BNT411 in total. In some embodiments, the composition is administered to the subject in an amount so as to provide about 2.4 μg/kg to about 9.6 μg/kg body weight (such as about 4.8 μg/kg to about 9.6 μg/kg body weight) of BNT411 or about 192 μg to about 768 μg (such as about 384 μg to about 768 μg) of BNT411 in total; and/or about 5.46×10⁻⁹ mol/kg body weight to about 21.8×10⁻⁹ mol/kg body weight (such as about 10.9×10⁻⁹ mol/kg body weight to about 21.8×10⁻⁹ mol/kg body weight) of BNT411 or about 437×10⁻⁹ mol to about 1747×10⁻⁹ mol (such as about 873×10⁻⁹ mol to about 1747×10⁻⁹ mol) of BNT411 in total. In some embodiments, the composition is administered to the subject in an amount so as to provide about 4.8 μg/kg body weight of BNT411 or about 384 μg of BNT411 in total; and/or about 10.9×10⁻⁹ mol/kg body weight of BNT411 or about 874×10⁻⁹ mol of BNT411 in total.

In a fourth aspect, the present disclosure provides a kit comprising (i) BNT411, i.e., a compound having the following formula

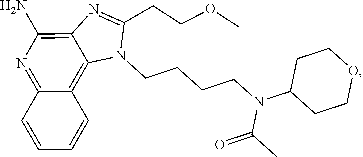 [SMILES: COCCc1nc2c(N)nc3ccccc3c2n1CCCCN(C(C)=O)C1CCOCC1]

or a pharmaceutically acceptable solvate or salt thereof, and (ii) one or more additional therapeutic agents selected from the group consisting of checkpoint inhibitors (CPIs), platinum-based chemotherapeutic agents, topoisomerase inhibitors, and combinations thereof.

In a fifth aspect, the present disclosure provides a kit of the fourth aspect for use in a method for reducing or preventing progression of a tumor or treating cancer in a subject. In some embodiments of the fifth aspect, BNT411 is administered to the subject in an amount of about 0.1 μg/kg to about 50 μg/kg body weight or about 8 μg to about 4000 μg in total; and/or about 0.23×10⁻⁹ mol/kg body weight to about 114×10⁻⁹ mol/kg body weight or about 18×10⁻⁹ mol to about 9100×10⁻⁹ mol in total. In some embodiments, BNT411 is administered to the subject in an amount of about 0.5 μg/kg to about 20 μg/kg body weight or about 40 μg to about 1600 μg in total; and/or about 1.14×10⁻⁹ mol/kg body weight to about 45.5×10⁻⁹ mol/kg body weight or about 91×10⁻⁹ mol to about 3640×10⁻⁹ mol in total. In some embodiments, BNT411 is administered to the subject in an amount of about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total. In some embodiments, BNT411 is administered to the subject in an amount of about 2.4 μg/kg to about 9.6 μg/kg body weight (such as about 4.8 μg/kg to about 9.6 μg/kg body weight) or about 192 μg to about 768 μg (such as about 384 μg to about 768 μg) in total; and/or about 5.46×10⁻⁹ mol/kg body weight to about 21.8×10⁻⁹ mol/kg body weight (such as about 10.9×10⁻⁹ mol/kg body weight to about 21.8×10⁻⁹ mol/kg body weight) or about 437×10⁻⁹ mol to about 1747×10⁻⁹ mol (such as about 873×10⁻⁹ mol to about 1747×10⁻⁹ mol) in total. In some embodiments, BNT411 is administered to the subject in an amount of about 4.8 μg/kg body weight or about 384 μg in total; and/or about 10.9×10⁻⁹ mol/kg body weight or about 874×10⁻⁹ mol in total.

### BRIEF DESCRIPTION OF THE FIGURES

FIG. 1. In vitro human PBMC stimulation assay. Stimulation of IFNa in human PBMC preparations from healthy donors with BNT411 (concentration range: 0.1 nM to 10 μM) or a comparative imidazoquinoline (resiquimod (R848; concentration range: 0.6 nM to 50 μM); 852A (concentration range: 0.6 nM to 50 μM); or imiquimod (R837; concentration range: 0.6 nM to 50 μM)). Data shown as mean±standard deviation (SD), n=5.

FIG. 2. In vivo cytokine release in BALB/c mice upon administration of BNT411. Female BALB/c mice were retro-orbitally administered with doses of 3, 5, 10, and 15 mg/kg BNT411. Blood samples were drawn 1 h after compound application and analyzed with mouse IFNa ELISA.

FIG. 3. Scheme of mode of action of BNT411.

FIG. 4. Anti-tumoral efficacy of BNT411 alone in a tumor mouse model. Mice (n=12 or 13 per group) subcutaneously inoculated with 5×10⁵ CT26 colon carcinoma cells were used as tumor mouse model. BNT411 was administered i.v. at a dose level of 3 mg/kg eight times with 4 to 5 days interval (on days 14, 19, 23, 28, 33, 37, 42, and 47). A control group (n=13) received vehicle alone. Adaptive immunity was evaluated by analyzing gp70-antigen-specific T cell populations in blood and splenocytes 3 and 5 weeks after the first study drug administration. (A) Dosing schedule. (B) Graph showing the tumor volumes vs. days after inoculation. Group mean tumor volumes for all live animals are shown. Additionally, tumor volumes of animals that were euthanized due to tumor load were carried forward for as long as they were larger than the group median tumor volume (SEM: standard error of the mean; LOCF: last observation carried forward values).

FIG. 5. Anti-tumoral efficacy of BNT411 alone and in combination with oxaliplatin in a tumor mouse model. Mice (n=12 or 13 per group) subcutaneously inoculated with 2.5×10⁵ CT26 colon carcinoma cells were used as tumor mouse model. Oxaliplatin was administered intraperitoneally (i.p.) (5 mg/kg, four times with 4 to 5 days between injections). BNT411 was administered i.v. at a dose level of 3 mg/kg eight times with 2 to 5 days interval between injections and at least 2 days after oxaliplatin therapy. Anti-tumor efficacy was evaluated by group mean tumor volumes in test versus control groups (vehicle only) on days 33 and 42 (at least 50% of test and control animals alive). IFNα levels in plasma were analyzed 1 h after first BNT411 administration and gp70+ T-cell populations in blood were analyzed on days 34 and 41. (A) Dosing schedule. (B) Graph showing the tumor volumes vs. days after inoculation. Group mean tumor volumes (±SEM (standard error of the mean)) for all live animals are shown. Treatment groups included nine to ten mice. Additionally, tumor volumes of animals that were euthanized due to tumor load were carried forward for as long as they were larger than the group median tumor volume (last observation carried forward (LOCF) values). Curves were discontinued for groups with less than 50% animals alive. (C) Graph showing IFNα serum level 60 min after first injection for the 4 groups (control (vehicle); BNT411 alone; oxaliplatin alone; BNT411 and oxaliplatin). IFNα levels in control and test groups are shown by vertical bars (±SEM), Statistical significance between groups is indicated by horizontal lines. P-value summary: ****=p≤0.0001; ***=p≤0.001; **=p≤0.01, *=p≤0.1.

FIG. 6. Anti-tumoral efficacy of BNT411 alone and in combination with an anti-PD-L1 antibody in a tumor mouse model. Mice (n=10 per group) subcutaneously inoculated with 5×10⁵ CT26 colon carcinoma cells were used as tumor mouse model. The anti-PD-L1 antibody anti-PD-L1-mIgG1e3 was administered intraperitoneally (i.p.) at a dose level of 200 μg per animal. BNT411 was administered i.v. at a dose level of 3 mg/kg. All treatments were delivered once every five days for a total of six doses. Body weight changes and tumor growth were assessed on study day 21 when there were at least nine animals alive in each group. (A) Dosing schedule. (B) Graph showing the tumor volumes vs. study day. Mean tumor volume±SEM (mm³) is depicted for each treatment. Last observations were carried forward until day 35 when only the animal who cleared the tumor was left. (C) Graph showing the effect of BNT411 and anti-PD-L1 combination treatment on tumor volume in the tumor mouse model (day 21 data). Individual animal values and mean±SEM are depicted for each treatment (*p≤0.05, **p≤0.01—Tukey's multiple comparison test).

FIG. 7. Release of certain cytokines upon treatment of whole blood from human donors with BNT411 at various concentrations. Whole blood drawn from healthy human volunteers was treated in vitro with BNT411 to investigate the induction of the following cytokines/chemokines: IFNα, IP-10, IL-6, and TNFα. BNT411 concentrations ranging from 0.1 nM (1×) to 7.5 μM (75000×). The fold increase to baseline is shown for each cytokine and BNT411 concentration tested.

FIG. 8. Release of certain cytokines from preparations obtained from whole blood of human patients treated with BNT411 at various dose levels. Mean maximum fold change (+SD) calculated from the highest cytokine value observed on day 1 in treatment cycle 1 relative to baseline. IFNα and IL-6 peaked 4 to 8 hours after treatment, IP-10 and TNFa peaked 2.5 to 8 hours after treatment. No meaningful cytokine changes were observed in DL1A, DL2A, or DL3A. IP-10⁺: The upper limit of detection of the assay was reached at DL6A.

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in some embodiments the suitable amount of BNT411 is about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total and in some other embodiments the tumor or cancer to be treated is a small cell lung cancer (SCLC), such as extensive-stage small cell lung cancer (ES-SCLC), then in further embodiments the suitable amount of BNT411 is about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total in order to treat a small cell lung cancer (SCLC), such as extensive-stage small cell lung cancer (ES-SCLC).

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Römpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The term “binding agent” in the context of the present disclosure refers to any agent capable of binding to desired antigens. In certain embodiments of the present disclosure, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In some embodiments, the binding agent is a synthetic construct comprising antigen-binding CDRs or variable regions.

As used herein, “immune checkpoint” refers to regulators of the immune system, and, in particular, co-stimulatory and inhibitory signals that regulate the amplitude and quality of T cell activity, such as the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2. A molecule which prevents the interaction between PD-1 and one or more of its binding partners, so as to remove T-cell dysfunction resulting from signaling on the PD-1 signaling axis—with a result being to restore or enhance T-cell function (e.g., proliferation, cytokine production, target cell killing)—, is also referred to herein as “PD-1 axis binding antagonist”. As used herein, a PD-1 axis binding antagonist includes a PD-1 binding antagonist, a PD-L1 binding antagonist and a PD-L2 binding antagonist.

Further immune checkpoint proteins (such as receptors and their ligands) that can be targeted according the disclosure are described and reviewed in Pardoll, D., Nature. 12:252-264, 2012. herein.

The terms “checkpoint inhibitor” (CPI) and “immune checkpoint (ICP) inhibitor” are used herein synonymously. The terms refer to molecules, such as binding agents, which totally or partially reduce, inhibit, interfere with or negatively modulate one or more checkpoint proteins or that totally or partially reduce, inhibit, interfere with or negatively modulate expression of one or more checkpoint proteins, like molecules, such as binding agents, which inhibit an immune checkpoint, in particular, which inhibit the inhibitory signal of an immune checkpoint. In some embodiments, the immune checkpoint inhibitor binds to one or more checkpoint proteins. In some embodiments, the immune checkpoint inhibitor binds to one or more molecules regulating checkpoint proteins. In some embodiments, the immune checkpoint inhibitor binds to precursors of one or more checkpoint proteins e.g., on DNA- or RNA-level. Any agent that functions as a checkpoint inhibitor according to the present disclosure can be used. The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In some embodiments, the checkpoint inhibitor can be any compound, such as any binding agent, which inhibits the inhibitory signal of an immune checkpoint. In some embodiments, the inhibitory signal is the interaction between PD-1 and PD-L1 and/or PD-L2. In some embodiments, the checkpoint inhibitor is at least one selected from the group consisting of PD-1 inhibitors, PD-L1 inhibitors, and PD-L2 inhibitors. In some embodiments, the checkpoint inhibitor may be a blocking antibody, such as a PD-1 blocking antibody, a PD-L1 blocking antibody, or a PD-L2 blocking antibody. Examples of a PD-1 blocking antibody include pembrolizumab, nivolumab, cemiplimab, and spartalizumab. Examples of a PD-L1 blocking antibody include atezolizumab, durvalumab, and avelumab.

The term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies.

The structure of immunoglobulins has been well characterized. See, e.g., Fundamental Immunology Ch. 7 (Paul, W., ed., 2^{nd} ed. Raven Press, N.Y. (1989)). Briefly, immunoglobulins generally comprise several chains, typically two identical heavy chains and two identical light chains which are linked via disulfide bonds. These chains are primarily composed of immunoglobulin domains or regions, such as the V_{L} or VL (variable light chain) domain/region, C_{L} or CL (constant light chain) domain/region, V_{H} or VH (variable heavy chain) domain/region, and the C_{H} or CH (constant heavy chain) domains/regions C_{H}1 (CH1), C_{H}2 (CH2), C_{H}3 (CH3), and C_{H}4 (CH4). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a VL and a CL. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P. Nucleic Acids Res., 38, D301-307 (2010); see also internet http address www.imgt.org. Unless otherwise stated or contradicted by context, reference to amino acid positions in the constant regions in the present disclosure is according to the EU-numbering (Edelman et al., Proc Natl Acad Sci USA. 1969 May; 63 (1): 78-85; Kabat et al., Sequences of Proteins of Immunological Interest, Fifth Edition. 1991 NIH Publication No. 91-3242).

There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics. Thus, classes of amino acids may be reflected in one or both of the following tables:

TABLE 1
Main classification based on structure and
general chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 2
Alternative Physical and Functional
Classifications of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, R, T, |
| | V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, |
| | R, S, P, and T |
| Flexible residues | Q, T, K, S, G, P, D, E, and R |

For the purposes of the present disclosure, “variants” of an amino acid sequence (peptide or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Substitution of one amino acid for another may be classified as a conservative or non-conservative substitution. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. In the context of the present disclosure, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues. Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

The term “amino acid corresponding to position . . . ” and similar expressions as used herein refer to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present disclosure.

The term “antibody” (Ab) in the context of the present disclosure refers to an immunoglobulin molecule, a fragment of an immunoglobulin molecule, or a derivative of either thereof, which has the ability to specifically bind to an antigen (in particular an epitope on an antigen) under typical physiological conditions, preferably with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3 (or CDR-H1, CDR-H2 and CDR-H3), the CDRs of a VL are termed LCDR1, LCDR2 and LCDR3 (or CDR-L1, CDR-L2 and CDR-L3). The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and components of the complement system such as C1q. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The variable regions of the heavy and light chains of the immunoglobulin molecule contain a binding domain that interacts with an antigen. The terms “binding region” and “antigen-binding region” are used herein interchangeably and refer to the region which interacts with the antigen and comprises both a VH region and a VL region. An antibody as used herein comprises not only monospecific antibodies, but also multispecific antibodies which comprise multiple, such as two or more, e.g., three or more, different antigen-binding regions.

As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO 2007/059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody; (v) a dAb fragment (Ward et al., Nature 341, 544-546 (1989)), which consists essentially of a VH domain and also called domain antibodies (Holt et al; Trends Biotechnol. 2003 November; 21 (11): 484-90); (vi) camelid or Nanobody molecules (Revets et al; Expert Opin Biol Ther. 2005 January; 5 (1): 111-24); and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

An antibody as generated can possess any isotype. As used herein, the term “isotype” refers to the immunoglobulin class (for instance IgG (such as IgG1, IgG2, IgG3, IgG4), IgD, IgA (such as IgA1, IgA2), IgE, IgM, or IgY) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody disclosed herein may be a sequence variant of a naturally-occurring IgG1 antibody, including variations in the constant regions.

IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The term “multispecific antibody” in the context of the present disclosure refers to an antibody having at least two different antigen-binding regions defined by different antibody sequences. In some embodiments, said different antigen-binding regions bind different epitopes on the same antigen. However, in some preferred embodiments, said different antigen-binding regions bind different target antigens. In some embodiments, the multispecific antibody is a “bispecific antibody” or “bs”. A multispecific antibody, such as a bispecific antibody, can be of any format, including any of the bispecific or multispecific antibody formats described herein below.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

The term “human antibody”, as used herein, is intended to include antibodies having variable and framework regions derived from human germline immunoglobulin sequences and a human immunoglobulin constant domain. The human antibodies disclosed herein may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generically for different kinds of modifications of antibodies, and processes for antibody engineering are well-known for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody may be performed by other methods than those described herein. Chimeric monoclonal antibodies for therapeutic applications in humans are developed to reduce anticipated antibody immunogenicity of non-human antibodies, e.g. rodent antibodies. They may typically contain non-human (e.g. murine or rabbit) variable regions, which are specific for the antigen of interest, and human constant antibody heavy and light chain domains. The terms “variable region” or “variable domain” as used in the context of chimeric antibodies, refer to a region which comprises the CDRs and framework regions of both the heavy and light chains of an immunoglobulin, as described below.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO 92/22653 and EP 0 629 240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

As used herein, a protein which is “derived from” another protein, e.g., a parent protein, means that one or more amino acid sequences of the protein are identical or similar to one or more amino acid sequences in the other or parent protein. For example, in an antibody, binding arm, antigen-binding region, constant region, or the like which is derived from another or a parent antibody, binding arm, antigen-binding region, or constant region, one or more amino acid sequences are identical or similar to those of the other or parent antibody, binding arm, antigen-binding region, or constant region. Examples of such one or more amino acid sequences include, but are not limited to, those of the VH and VL CDRs and/or one or more or all of the framework regions, VH, VL, CL, hinge, or CH regions. For example, a humanized antibody can be described herein as “derived from” a non-human parent antibody, meaning that at least the VL and VH CDR sequences are identical or similar to the VH and VL CDR sequences of said non-human parent antibody. A chimeric antibody can be described herein as being “derived from” a non-human parent antibody, meaning that typically the VH and VL sequences may be identical or similar to those of the non-human parent antibody. Another example is a binding arm or an antigen-binding region which may be described herein as being “derived from” a particular parent antibody, meaning that said binding arm or antigen-binding region typically comprises identical or similar VH and/or VL CDRs, or VH and/or VL sequences to the binding arm or antigen-binding region of said parent antibody. As described elsewhere herein, however, amino acid modifications such as mutations can be made in the CDRs, constant regions or elsewhere in the antibody, binding arm, antigen-binding region or the like, to introduce desired characteristics. When used in the context of one or more sequences derived from a first or parent protein, a “similar” amino acid sequence preferably has a sequence identity of at least about 50%, such as at least about 60%, at least about 70%, at least about 80%, at least about 90%, at least about 95%, or at least about 97%, 98% or 99%.

Non-human antibodies can be generated in a number of different species, such as mouse, rabbit, chicken, guinea pig, llama and goat.

Monoclonal antibodies can be produced by a variety of techniques, including conventional monoclonal antibody methodology, e.g., the standard somatic cell hybridization technique of Kohler and Milstein, Nature 256:495 (1975). Other techniques for producing monoclonal antibodies can be employed, e.g., viral or oncogenic transformation of B-lymphocytes or phage display techniques using libraries of antibody genes, and such methods are well known to a person skilled in the art.

Hybridoma production in such non-human species is a very well established procedure. Immunization protocols and techniques for isolation of splenocytes of immunized animals/non-human species for fusion are known in the art. Fusion partners (e.g., murine myeloma cells) and fusion procedures are also known.

When used herein, unless contradicted by context, the term “Fab-arm” or “arm” refers to one heavy chain-light chain pair and is used interchangeably with “half molecules” herein.

The term “binding arm comprising an antigen-binding region” means an antibody molecule or fragment that comprises an antigen-binding region. Thus, a binding arm can comprise, e.g., the six VH and VL CDR sequences, the VH and VL sequences, a Fab or Fab′ fragment, or a Fab-arm.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain. In some embodiments, the term “Fc region”, as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

The term “hinge region” as used herein refers to the hinge region of an immunoglobulin heavy chain. Thus, for example, the hinge region of a human IgG1 antibody corresponds to amino acids 216-230 according to the EU numbering as set forth in Kabat (Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example, the CH1 region of a human IgG1 antibody corresponds to amino acids 118-215 according to the EU numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example, the CH2 region of a human IgG1 antibody corresponds to amino acids 231-340 according to the EU numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example, the CH3 region of a human IgG1 antibody corresponds to amino acids 341-447 according to the EU numbering as set forth in Kabat (ibid). However, the CH3 region may also be any of the other subtypes as described herein.

The term “monovalent antibody” means in the context of the present disclosure that an antibody molecule is capable of binding a single molecule of the antigen, and thus is not capable of antigen cross-linking.

Based on the type of receptor through which they signal, interferons are typically divided among three classes: type I interferon, type II interferon, and type III interferon. All type I interferons bind to a specific cell surface receptor complex known as the IFN-α/β receptor (IFNAR) that consists of IFNAR1 and IFNAR2 chains.

The type I interferons present in humans are IFNα, IFNβ, IFNε, IFNε and IFNω. In general, type I interferons are produced when the body recognizes a virus that has invaded it. They are produced by fibroblasts and monocytes. Once released, type I interferons bind to specific receptors on target cells, which leads to expression of proteins that will prevent the virus from producing and replicating its RNA and DNA. The IFNβ proteins are produced in large quantities by fibroblasts. They have antiviral activity that is involved mainly in innate immune response. Two types of IFNβ have been described, IFNβ1 and IFNβ3. The natural and recombinant forms of IFNβ1 have antiviral, antibacterial, and anticancer properties. According to the disclosure, a type I interferon is preferably IFNα. “IFNa” or “IFNα” are used herein interchangeably and mean interferon alfa (Uniprot #P01563). Dendritic cells are a major source of IFNa and the protein is involved in the innate immune response to viral infection, but also cell differentiation and anti-tumor responses.

Type II interferon (IFNγ in humans) is also known as immune interferon and is activated by IL12. Furthermore, type II interferons are released by cytotoxic T cells and T helper cells.

Type III interferons signal through a receptor complex consisting of IL10R2 (also called CRF2-4) and IFNLR1 (also called CRF2-12). Although discovered more recently than type I and type II IFNs, recent information demonstrates the importance of type III IFNs in some types of virus or fungal infections.

“IP-10” as used herein means interferon gamma induced protein 10 (also known as 10 kDa Interferon-Gamma-Induced Protein or CXCL10) (Uniprot #P02778). IP-10 is induced in a variety of cells in response to interferon gamma. IP-10 inhibits bone marrow colony formation and angiogenesis, stimulates NK and T cell migration, regulates T cell maturation, and modulates adhesion molecule expression.

“IFN gamma” as used herein means interferon gamma (Uniprot #P01579). Its major functions are to activate macrophages and to increase the expression of class II MHC on APCs. IFN gamma stimulated macrophages are more phagocytic, they are more capable of killing intracellular pathogens and they have increased ability to present antigen.

“IL6” and “IL-6” are used herein interchangeably and mean interleukin-6 (Uniprot #P05231). IL-6 is a cytokine released by leukocytes in response to a number of inciting stimuli. In addition to its role as an acute phase reactant and endogenous pyrogen, IL-6 is also involved in B-cell differentiation into plasma cells. IL-6 is usually not detected in normal serum, plasma, cerebrospinal fluid (CSF), or joint fluid. Elevated levels are observed in a variety of inflammatory processes, including infections (endotoxemia) and collagen vascular diseases.

“TNFa” and “TNFα” are used herein interchangeably and mean tumor necrosis factor alpha (Uniprot #P01375). TNFa is secreted by macrophages, monocytes, neutrophils, T-cells and NK-cells following many different stimuli including interferon, IL-2, GM-CSF, bradykinin, immune complexes, inhibitors of cyclooxygenase and PAF (platelet activating factor).

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely-related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

Two antibodies have the “same specificity” if they bind to the same antigen and to the same epitope. Whether an antibody to be tested recognizes the same epitope as a certain antigen-binding antibody, i.e., the antibodies bind to the same epitope, may be tested by different methods well known to a person skilled in the art.

The competition between the antibodies can be detected by a cross-blocking assay. For example, a competitive ELISA assay may be used as a cross-blocking assay. E.g., target antigen may be coated on the wells of a microtiter plate and antigen-binding antibody and candidate competing test antibody may be added. The amount of the antigen-binding antibody bound to the antigen in the well indirectly correlates with the binding ability of the candidate competing test antibody that competes therewith for binding to the same epitope. Specifically, the larger the affinity of the candidate competing test antibody is for the same epitope, the smaller the amount of the antigen-binding antibody bound to the antigen-coated well. The amount of the antigen-binding antibody bound to the well can be measured by labeling the antibody with detectable or measurable labeling substances.

An antibody competing for binding to an antigen with another antibody, e.g., an antibody comprising heavy and light chain variable regions as described herein, or an antibody having the specificity for an antigen of another antibody, e.g., an antibody comprising heavy and light chain variable regions as described herein, may be an antibody comprising variants of said heavy and/or light chain variable regions as described herein, e.g. modifications in the CDRs and/or a certain degree of identity as described herein.

An “isolated multispecific antibody” as used herein is intended to refer to a multispecific antibody which is substantially free of other antibodies having different antigenic specificities (for instance an isolated bispecific antibody that specifically binds to PD-1 and a heterologous protein is substantially free of monospecific antibodies that specifically bind to PD-1 or the heterologous protein).

The term “monoclonal antibody” as used herein refers to a preparation of antibody molecules of single molecular composition. A monoclonal antibody composition displays a single binding specificity and affinity for a particular epitope.

The present disclosure also describes multispecific antibodies, such as bispecific antibodies, comprising functional variants of the VL regions, VH regions, or one or more CDRs of the bispecific antibodies disclosed herein. A functional variant of a VL, VH, or CDR used in the context of a bispecific antibody still allows each antigen-binding region of the bispecific antibody to retain at least a substantial proportion (at least about 50%, 60%, 70%, 80%, 90%, 95% or more) of the affinity and/or the specificity/selectivity of the parent bispecific antibody and in some cases such a bispecific antibody may be associated with greater affinity, selectivity and/or specificity than the parent bispecific antibody.

Such functional variants typically retain significant sequence identity to the parent bispecific antibody. The percent identity between two sequences is a function of the number of identical positions shared by the sequences (i.e., % homology=# of identical positions/total #of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

Exemplary variants include those which differ from the VH and/or VL and/or CDRs of the parent sequences mainly by conservative substitutions; for example, 12, such as 11, 10, 9, 8, 7, 6, 5, 4, 3, 2 or 1 of the substitutions in the variant are conservative amino acid residue replacements.

In the context of the present disclosure, conservative substitutions may be defined by substitutions within the classes of amino acids as defined in tables 1 and 2.

The “Programmed Death-1 (PD-1)” receptor refers to an immuno-inhibitory receptor belonging to the CD28 family. PD-1 (also known as CD279 or SLEB2) is expressed predominantly on previously activated T cells in vivo, and binds to two ligands, PD-L1 (also known as B7-H1, B7-4, CD274, or B7-H) and PD-L2 (also known as B7-DC, Btdc, or CD273). The term “PD-1” as used herein includes human PD-1 (hPD-1), variants, isoforms, and species homologs of hPD-1, and analogs having at least one common epitope with hPD-1. The sequence of human PD-1 is known (cf., e.g., Genbank accession no. AAC51773.1). “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, such as macaque (cynomolgus monkey), African elephant, wild boar and mouse PD-L1 (cf., e.g., Genbank accession no. NP_054862.1, XP_005581836, XP_003413533, XP_005665023 and NP_068693, respectively), and analogs having at least one common epitope with hPD-L1. The sequence of human PD-L1 is known (cf., e.g., Genbank accession no. NP_054862, wherein amino acids 1-18 are predicted to be a signal peptide). The sequence of macaque (cynomolgus monkey) PD-L1 is shown in Genbank accession no. XP_005581836, wherein amino acids 1-18 are predicted to be a signal peptide. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins which in some embodiments is modulated according to the present disclosure is typically the regulation of T cell activation, T cell proliferation and/or T cell function. Immune checkpoint proteins thus regulate and maintain self-tolerance and the duration and amplitude of physiological immune responses. Many of the immune checkpoint proteins belong to the B7: CD28 family or to the tumor necrosis factor receptor (TNFR) super family and, by binding to specific ligands, activate signaling molecules that are recruited to the cytoplasmic domain (Suzuki et al., 2016, Jap J Clin Onc, 46:191-203).

As used herein, the term “immune checkpoint modulator” or “checkpoint modulator” refers to a molecule or to a compound that modulates the function of one or more checkpoint proteins. Immune checkpoint modulators are typically able to modulate self-tolerance and/or the amplitude and/or the duration of the immune response. Preferably, the immune checkpoint modulator modulates the function of one or more human checkpoint proteins and is, thus, a “human checkpoint modulator”. Specifically, the human checkpoint modulator is an immune checkpoint inhibitor.

The term “dysfunctional”, as used herein, refers to an immune cell that is in a state of reduced immune responsiveness to antigen stimulation. Dysfunctional includes unresponsive to antigen recognition and impaired capacity to translate antigen recognition into downstream T cell effector functions, such as proliferation, cytokine production (e.g., IL-2) and/or target cell killing.

The term “anergy”, as used herein, refers to the state of unresponsiveness to antigen stimulation resulting from incomplete or insufficient signals delivered through the T cell receptor (TCR). T cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T cells do not undergo clonal expansion and/or acquire effector functions.

The term “exhaustion”, as used herein, refers to immune cell exhaustion, such as T cell exhaustion as a state of T cell dysfunction that arises from sustained TCR signaling that occurs during many chronic infections and cancer. It is distinguished from anergy in that it arises not through incomplete or deficient signaling, but from sustained signaling. Exhaustion is defined by poor effector function, sustained expression of inhibitory receptors and a transcriptional state distinct from that of functional effector or memory T cells. Exhaustion prevents optimal control of diseases (e.g., infection and tumors). Exhaustion can result from both extrinsic negative regulatory pathways (e.g., immunoregulatory cytokines) as well as cell intrinsic negative regulatory pathways (inhibitory immune checkpoint pathways, such as described herein).

“Enhancing T cell function” means to induce, cause or stimulate a T cell to have a sustained or amplified biological function, or renew or reactivate exhausted or inactive T cells. Examples of enhancing T cell function include increased secretion of y-interferon from CD8+ T cells, increased proliferation, increased antigen responsiveness (e.g., tumor clearance) relative to such levels before the intervention. In some embodiments, the level of enhancement is as least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 200%, or more. Manners of measuring this enhancement are known to one of ordinary skill in the art.

The term “inhibitory nucleic acid” or “inhibitory nucleic acid molecule” as used herein refers to a nucleic acid molecule, e.g., DNA or RNA, that totally or partially reduces, inhibits, interferes with or negatively modulates one or more checkpoint proteins. Inhibitory nucleic acid molecules include, without limitation, oligonucleotides, siRNA, shRNA, antisense DNA or RNA molecules, and aptamers (e.g., DNA or RNA aptamers).

The term “oligonucleotide” as used herein refers to a nucleic acid molecule that is able to decrease protein expression, in particular expression of a checkpoint protein, such as the checkpoint proteins described herein. Oligonucleotides are short DNA or RNA molecules, typically comprising from 2 to 50 nucleotides. Oligonucleotides maybe single-stranded or double-stranded. A checkpoint inhibitor oligonucleotide may be an antisense-oligonucleotide.

Antisense-oligonucleotides are single-stranded DNA or RNA molecules that are complementary to a given sequence, in particular to a sequence of the nucleic acid sequence (or a fragment thereof) of a checkpoint protein. Antisense RNA is typically used to prevent protein translation of mRNA, e.g., of mRNA encoding a checkpoint protein, by binding to said mRNA. Antisense DNA is typically used to target a specific, complementary (coding or non-coding) RNA. If binding takes place, such a DNA/RNA hybrid can be degraded by the enzyme RNase H. Moreover, morpholino antisense oligonucleotides can be used for gene knockdowns in vertebrates. For example, Kryczek et al., 2006 (J Exp Med, 203:871-81) designed B7-H4-specific morpholinos that specifically blocked B7-H4 expression in macrophages, resulting in increased T cell proliferation and reduced tumor volumes in mice with tumor associated antigen (TAA)-specific T cells.

The terms “siRNA” or “small interfering RNA” or “small inhibitory RNA” are used interchangeably herein and refer to a double-stranded RNA molecule with a typical length of 20-25 base pairs that interferes with expression of a specific gene, such as a gene coding for a checkpoint protein, with a complementary nucleotide sequence. In some embodiments, siRNA interferes with mRNA therefore blocking translation, e.g., translation of an immune checkpoint protein. Transfection of exogenous siRNA may be used for gene knockdown, however, the effect maybe only transient, especially in rapidly dividing cells. Stable transfection may be achieved, e.g., by RNA modification or by using an expression vector. Useful modifications and vectors for stable transfection of cells with siRNA are known in the art, siRNA sequences may also be modified to introduce a short loop between the two strands resulting in a “small hairpin RNA” or “shRNA”. shRNA can be processed into a functional siRNA by Dicer. shRNA has a relatively low rate of degradation and turnover. Accordingly, the immune checkpoint inhibitor may be a shRNA.

The term “aptamer” as used herein refers to a single-stranded nucleic acid molecule, such as DNA or RNA, typically in a length of 25-70 nucleotides that is capable of binding to a target molecule, such as a polypeptide. In some embodiments, the aptamer binds to an immune checkpoint protein such as the immune checkpoint proteins described herein. For example, an aptamer according to the disclosure can specifically bind to an immune checkpoint protein or polypeptide, or to a molecule in a signaling pathway that modulates the expression of an immune checkpoint protein or polypeptide. The generation and therapeutic use of aptamers is well known in the art (see, e.g., U.S. Pat. No. 5,475,096).

The terms “small molecule inhibitor” or “small molecule” are used interchangeably herein and refer to a low molecular weight organic compound, usually up to 1000 daltons, that totally or partially reduces, inhibits, interferes with, or negatively modulates one or more checkpoint proteins as described above. Such small molecular inhibitors are usually synthesized by organic chemistry, but may also be isolated from natural sources, such as plants, fungi, and microbes. The small molecular weight allows a small molecule inhibitor to rapidly diffuse across cell membranes. For example, various A2AR antagonists known in the art are organic compounds having a molecular weight below 500 daltons.

The term “cell based therapy” refers to the transplantation of cells (e.g., T lymphocytes, dendritic cells, or stem cells) expressing an immune checkpoint inhibitor into a subject for the purpose of treating a disease or disorder (e.g., a cancer disease).

The term “oncolytic virus” as used herein, refers to a virus capable of selectively replicating in and slowing the growth or inducing the death of a cancerous or hyperproliferative cell, either in vitro or in vivo, while having no or minimal effect on normal cells. An oncolytic virus for the delivery of an immune checkpoint inhibitor comprises an expression cassette that may encode an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. The oncolytic virus preferably is replication competent and the expression cassette is under the control of a viral promoter, e.g., synthetic early/late poxvirus promoter. Exemplary oncolytic viruses include vesicular stomatitis virus (VSV), rhabdoviruses (e.g., picornaviruses such as Seneca Valley virus; SVV-001), coxsackievirus, parvovirus, Newcastle disease virus (NDV), herpes simplex virus (HSV; OncoVEX GMCSF), retroviruses (e.g., influenza viruses), measles virus, reovirus, Sinbis virus, vaccinia virus, as exemplarily described in WO 2017/209053 (including Copenhagen, Western Reserve, Wyeth strains), and adenovirus (e.g., Delta-24, Delta-24-RGD, ICOVIR-5, ICOVIR-7, Onyx-015, ColoAd1, H101, AD5/3-D24-GMCSF). Generation of recombinant oncolytic viruses comprising a soluble form of an immune checkpoint inhibitor and methods for their use are disclosed in WO 2018/022831, herein incorporated by reference in its entirety. Oncolytic viruses can be used as attenuated viruses.

“Treatment cycle” is herein defined as the time period, within the effects of separate dosages of a compound (such as BNT411 and/or one or more additional therapeutic agents) add on due to its pharmacodynamics, or in other words the time period after the subject's body is essentially cleared from the administrated biding agent. Multiple small doses in a small time window, e.g. within 2-24 few hours, such as 2-12 hours or on the same day, might be equal to a larger single dose.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications. In some embodiments, “treatment” refers to the administration of an effective amount of a therapeutically active compound (such as BNT411 and/or one or more additional therapeutic agents of the present disclosure) with the purpose of easing, ameliorating, arresting or eradicating (curing) symptoms or disease states.

The resistance to, failure to respond to and/or relapse from treatment with a compound (such as BNT411 and/or one or more additional therapeutic agents) of the present disclosure may be determined according to the Response Evaluation Criteria in Solid Tumors; version 1.1 (RECIST Criteria v1.1). The RECIST Criteria are set forth in the table below (LD: longest dimension).

TABLE 3
Definition of Response (RECIST Criteria v1.1)

| | Category | Criteria |
|---|---|---|
| Based | Complete | Disappearance of all target lesions. Any |
| on | Response | pathological lymph nodes must have |
| target | (CR) | reduction in short axis to <10 mm. |
| lesions | Partial | ≥30% decrease in the sum of the LD of target |
| | Response | lesions, taking as reference the |
| | (PR) | baseline sum LD. |
| | Stable | Neither sufficient shrinkage to qualify for PR |
| | Disease | nor sufficient increase to qualify for PD, |
| | (SD) | taking as reference the smallest sum of |
| | | LDs since the treatment started. |
| | Progressive | ≥20% increase in the sum of the LDs of target |
| | Disease | lesions, taking as reference the smallest sum of the |
| | (PD) | LDs recorded since the treatment started or the |
| | | appearance of one or more new lesions. |
| Based- | CR | Disappearance of all non-target lesions and |
| on non | | normalization of tumor marker level. |
| target | | All lymph nodes must be non- |
| | | pathological in size (<10 mm short axis). |
| lesions | SD | Persistence of one or more non-target lesion(s) |
| | | or/and maintenance of tumor marker level |
| | | above the normal limits. |
| | PD | Appearance of one or more new lesions and/or |
| | | unequivocal progression of |
| | | existing non-target lesions. |

The “best overall response” is the best response recorded from the start of the treatment until disease progression/recurrence (the smallest measurements recorded since the treatment started will be used as the reference for PD). Subjects with CR or PR are considered to be objective response. Subjects with CR, PR or SD are considered to be in disease control. Subjects with NE are counted as non-responders. The best overall response is the best response recorded from the start of the treatment until disease progression/recurrence (the smallest measurements recorded since the treatment started will be used as the reference for PD). Subjects with CR, PR or SD are considered to be in disease control. Subjects with NE are counted as non-responders.

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from day 1 in cycle 1 to the first documented progression or death due to any cause.

“Overall survival (OS)” is defined as the number of days from day 1 in cycle 1 to death due to any cause. If a subject is not known to have died, then OS will be censored at the latest date the subject was known to be alive (on or before the cut-off date).

In the context of the present disclosure, the term “treatment regimen” refers to a structured treatment plan designed to improve and maintain health.

The term “effective amount” or “therapeutically effective amount” refers to an amount effective, at dosages and for periods of time necessary, to achieve a desired therapeutic result. A therapeutically effective amount of a compound (such as BNT411, an antibody or a fragment of an antibody) may vary according to factors such as the disease state, age, sex, and weight of the individual, and the ability of the compound to elicit a desired response in the individual. A therapeutically effective amount is also one in which any toxic or detrimental effects of the compound are outweighed by the therapeutically beneficial effects. In the case that a reaction in a patient is insufficient with an initial dose, higher doses (or effectively higher doses achieved by a different, more localized route of administration) may be used. In case that unwanted side effects occur in a patient with a dose, lower doses (or effectively lower doses achieved by a different, more localized route of administration) may be used.

As used herein, the term “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease.

The term “cancer” according to the present disclosure comprises leukemias, seminomas, melanomas, sarcomas, myelomas (such as multiple myeloma), teratomas, lymphomas (such as Hodgkin's lymphoma, non-Hodgkin's lymphoma), mesotheliomas, neuroblastomas, gliomas, myelodysplastic syndromes, rectal cancer, endometrial cancer, ureteral cancer, kidney cancer, renal cancer, urothelial cancer, bladder cancer, esophageal cancer, adrenal cancer, adrenocortical cancer, thyroid cancer, blood cancer, skin cancer (such as Merkel cell carcinoma), cancer of the brain, cervical cancer, malignant solitary fibrous tumor, intestinal cancer, liver cancer, thymoma and thymic carcinoma, colon cancer, stomach cancer, intestine cancer, head and neck cancer, gastric cancer, gastrointestinal cancer, lymph node cancer, esophagus cancer, colorectal cancer, pancreas cancer, ear, nose and throat (ENT) cancer, breast cancer, prostate cancer, penile cancer, cancer of the uterus, ovarian cancer and lung cancer (e.g., non-small cell lung cancer (NSCLC) or small cell lung cancer (SCLC)) and the metastases thereof. Examples thereof are lung carcinomas, mamma carcinomas, prostate carcinomas, colon carcinomas, renal cell carcinomas, cervical carcinomas, or metastases of the cancer types or tumors described above.

The term “cancer” according to the present disclosure also comprises cancer metastases. By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In some embodiments, the term “metastasis” according to the present disclosure relates to “distant metastasis” which relates to a metastasis which is remote from the primary tumor and the regional lymph node system.

Terms such as “reduce”, “inhibit”, “interfere”, and “negatively modulate” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in some embodiments relate to an increase or enhancement, for example, by at least about 5%, at least about 10%, at least about 15%, at least about 20%, at least about 25%, at least about 30%, at least about 40%, at least about 50%, at least about 75%, or at least about 100%, in the level.

“Physiological pH” as used herein refers to a pH of about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” refers to weight percent, which is a unit of concentration measuring the amount of a substance in grams (g) expressed as a percent of the total weight of the total composition in grams (g).

The term “freezing” relates to the solidification of a liquid, usually with the removal of heat.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., e.g., from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide or polypeptide), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

According to the present disclosure, a part or fragment of a peptide or polypeptide preferably has at least one functional property of the peptide or polypeptide from which it has been derived. Such functional properties comprise a pharmacological activity, the interaction with other peptides or polypeptides, an enzymatic activity, the interaction with antibodies, and the selective binding of nucleic acids. E.g., a pharmacological active fragment of a peptide or polypeptide has at least one of the pharmacological activities of the peptide or polypeptide from which the fragment has been derived. A part or fragment of a peptide or polypeptide preferably comprises a sequence of at least 6, in particular at least 8, at least 10, at least 12, at least 15, at least 20, at least 30 or at least 50, consecutive amino acids of the peptide or polypeptide. A part or fragment of a peptide or polypeptide preferably comprises a sequence of up to 8, in particular up to 10, up to 12, up to 15, up to 20, up to 30 or up to 55, consecutive amino acids of the peptide or polypeptide.

“Variant”, as used herein and with reference to an amino acid sequence (peptide or polypeptide), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

In some embodiment, the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the degree of similarity or identity is given for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference amino acid sequence consists of 200 amino acid residues, the degree of identity is given for at least about 100, at least about 120, at least about 140, at least about 160, at least about 180, or about 200 amino acid residues, in some embodiments continuous amino acid residues. In some embodiments, the degree of similarity or identity is given for the entire length of the reference sequence.

Homologous amino acid sequences exhibit according to the present disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or polypeptides having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In some embodiments, a fragment or variant of an amino acid sequence (peptide or polypeptide) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide or polypeptide) “derived from” a designated amino acid sequence (peptide or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In some embodiments, “isolated” means altered or removed from the natural state. For example, a nucleic acid, peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell.

The term “genetic modification” or simply “modification” includes the transfection of cells with nucleic acid.

The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the present disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded peptide or polypeptide.

According to the present disclosure, an analog of a peptide or polypeptide is a modified form of said peptide or polypeptide from which it has been derived and has at least one functional property of said peptide or polypeptide. E.g., a pharmacological active analog of a peptide or polypeptide has at least one of the pharmacological activities of the peptide or polypeptide from which the analog has been derived. Such modifications include any chemical modification and comprise single or multiple substitutions, deletions and/or additions of any molecules associated with the peptide or polypeptide, such as carbohydrates, lipids and/or peptides or polypeptides. In some embodiments, “analogs” of peptides or polypeptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said peptides and polypeptides.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or polypeptide, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In some embodiments, an antigen is a disease-associated antigen, such as a tumor antigen, a viral antigen, or a bacterial antigen, or an epitope derived from such antigen.

According to the present disclosure, any suitable antigen may be used, which is a candidate for an immune response, wherein the immune response may be both a humoral as well as a cellular immune response. In the context of some embodiments of the present disclosure, the antigen is preferably presented by a cell, preferably by an antigen presenting cell, in the context of MHC molecules, which results in an immune response against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In some preferred embodiments, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4+ and CD8+ lymphocytes, in particular via the modulation of the activity of a T-cell receptor.

The term “tumor antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, α1-fetoprotein, isoferritin, and fetal sulphoglycoprotein, α2-H-ferroprotein and γ-fetoprotein, as well as various virus tumor antigens. According to the present disclosure, a tumor antigen preferably comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies T cells or B cells, in particular when presented in the context of MHC molecules. In some embodiments, “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen-binding peptide (in other words, the amino acid residue is within the footprint of the specifically antigen-binding peptide).

An epitope of a protein preferably comprises a continuous or discontinuous portion of said protein and is preferably between about 5 and about 100, preferably between about 5 and about 50, more preferably between about 8 and about 0, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present disclosure is a T cell epitope.

Terms such as “epitope”, “fragment of an antigen”, “immunogenic peptide” and “antigen peptide” are used interchangeably herein and preferably relate to an incomplete representation of an antigen which is preferably capable of eliciting an immune response against the antigen or a cell expressing or comprising and preferably presenting the antigen. Preferably, the terms relate to an immunogenic portion of an antigen. Preferably, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide or polypeptide antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or polypeptide antigen can be any peptide or polypeptide that can induce or increase the ability of the immune system to develop antibodies and T cell responses to the peptide or polypeptide.

In some embodiments, vaccine antigen, i.e., an antigen whose inoculation into a subject induces an immune response, is recognized by an immune effector cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell. In some embodiments, an antigen is presented by a diseased cell (such as tumor cell or an infected cell). In some embodiments, an antigen receptor is a TCR which binds to an epitope of an antigen presented in the context of MHC. In some embodiments, binding of a TCR when expressed by T cells and/or present on T cells to an antigen presented by cells such as antigen presenting cells results in stimulation, priming and/or expansion of said T cells. In some embodiments, binding of a TCR when expressed by T cells and/or present on T cells to an antigen presented on diseased cells results in cytolysis and/or apoptosis of the diseased cells, wherein said T cells preferably release cytotoxic factors, e.g., perforins and granzymes.

In some embodiments, an antigen receptor is an antibody or B cell receptor which binds to an epitope in an antigen. In some embodiments, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

The term “target” shall mean an agent such as a cell or tissue which is a target for an immune response such as a cellular immune response. Targets include cells that present an antigen or an antigen epitope, i.e., a peptide fragment derived from an antigen. In some embodiments, the target cell is a cell expressing an antigen and preferably presenting said antigen with class I MHC.

“Antigen processing” refers to the degradation of an antigen into processing products which are fragments of said antigen (e.g., the degradation of a protein into peptides) and the association of one or more of these fragments (e.g., via binding) with MHC molecules for presentation by cells, preferably antigen-presenting cells to specific T-cells.

By “antigen-responsive CTL” is meant a CD8⁺ T-cell that is responsive to an antigen or a peptide derived from said antigen, which is presented with class I MHC on the surface of antigen presenting cells.

According to the present disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and preferably refers to a cellular immune response, a humoral immune response, or both. According to the present disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, preferably CD4⁺ and CD8⁺ T-lymphocytes, more preferably CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, preferably the induction of a cellular immune response, a humoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. Preferably, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The terms “vaccination” and “immunization” describe the process of treating an individual for therapeutic or prophylactic reasons and relate to the procedure of administering one or more immunogen(s) or antigen(s) or derivatives thereof, e.g., in the form of RNA coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, preferably MHC class I and/or MHC class II molecules, most preferably MHC class I molecules.

In the context of the present disclosure, the term “transcription” relates to a process, wherein the genetic code in a DNA sequence is transcribed into RNA (especially mRNA). Subsequently, the RNA (especially mRNA) may be translated into peptide or polypeptide.

The term “expression” as used herein is defined as the transcription and/or translation of a particular nucleotide sequence. With respect to RNA, the term “expression” or “translation” relates to the process in the ribosomes of a cell by which a strand of mRNA directs the assembly of a sequence of amino acids to make a peptide or polypeptide.

The term “optional” or “optionally” as used herein means that the subsequently described event, circumstance or condition may or may not occur, and that the description includes instances where said event, circumstance, or condition occurs and instances in which it does not occur.

As used herein “endogenous” refers to any material from or produced inside an organism, cell, tissue or system.

As used herein, the terms “linked”, “fused”, or “fusion” are used interchangeably. These terms refer to the joining together of two or more elements or components or domains.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. One example of a disease is a tumor or cancer.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably. Similarly, the term “method for preventing” in the context of progression of a disease, such as progression of a tumor or cancer, relates to any method that is intended to prevent the disease from progressing in an individual.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject, especially a diseased human.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions/formulations of the present disclosure or be shipped together with a container which contains the compositions/formulations. Alternatively, the instructional material may be shipped separately from the container with the intention that the instructional material and the compositions be used cooperatively by the recipient.

Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

In the present specification, a structural formula of a compound may represent a certain isomer of said compound. It is to be understood, however, that the present disclosure includes all isomers such as geometrical isomers, optical isomers based on an asymmetrical carbon, stereoisomers, tautomers and the like which occur structurally and isomer mixtures and is not limited to the description of the formula. Furthermore, in the present specification, a structural formula of a compound may represent a specific salt and/or solvate of said compound. It is to be understood, however, that the present disclosure includes all salts (e.g., pharmaceutically acceptable salts) and solvates (e.g., hydrates) and is not limited to the description of the specific salt and/or solvate.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (+). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimentional forms of (hetero)cyclic rings, such as chair, half-chair, boat, and twist-boat forms of cyclohexane.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, dimethylsulfoxide (DMSO), and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate wherein the solvent is water.

In isotopically labeled compounds one or more atoms are replaced by a corresponding atom having the same number of protons but differing in the number of neutrons. For example, a hydrogen atom may be replaced by a deuterium or tritium atom. Exemplary isotopes which can be used in the present disclosure include deuterium, tritium, ¹¹C, ¹³C, ¹⁴C, ¹⁵N, ¹⁸F, ³²P, ³²S, ³⁵S, ³⁶Cl, and ¹²⁵I.

The term “dose” as used herein refers in general to a “dose amount” which relates to the amount of a compound (such as BNT411) administered per administration, i.e., per dosing.

An “adverse event” or AE is any unfavorable and unintended sign (including an abnormal laboratory finding), symptom, or disease temporally associated with the use of a medical treatment or procedure that may or may not be considered related to the medical treatment or procedure. In some embodiments, AEs are classified using “Common Terminology Criteria for Adverse Events v3.0 (CTCAE)” (published: Aug. 9, 2006).

### Aspects and Embodiments of the Present Disclosure

In a first aspect, the present disclosure provides BNT411 (including a pharmaceutically acceptable solvate or salt thereof) for use in a method for reducing or preventing progression of a tumor or treating cancer in a subject, said method comprising administering to said subject BNT411 in a suitable amount.

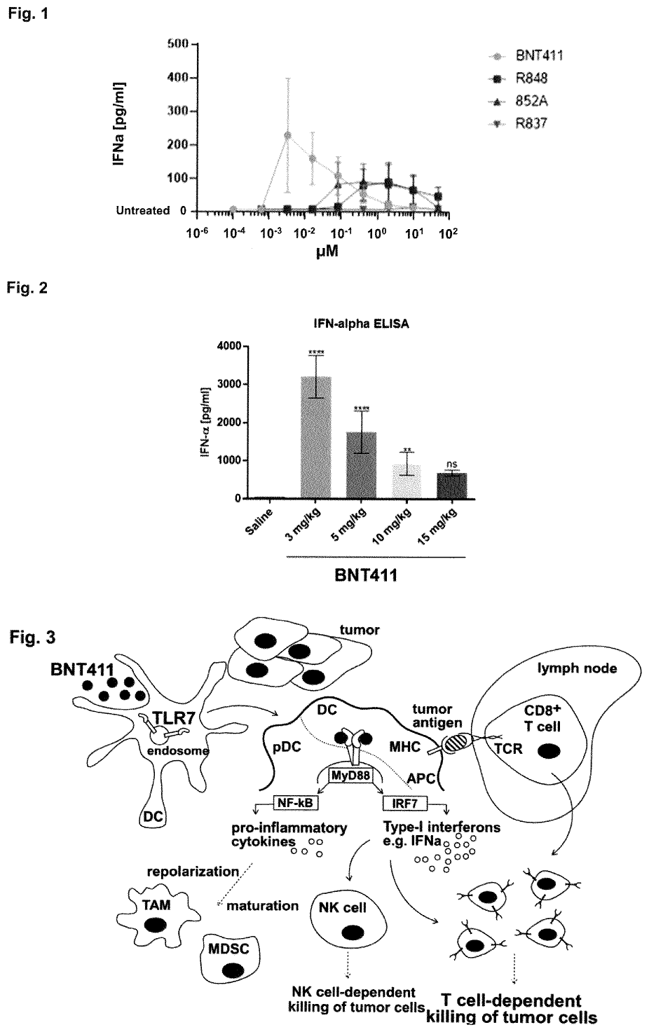

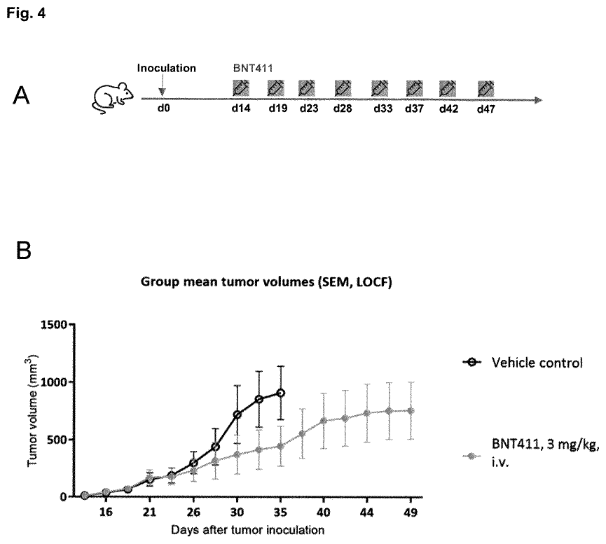

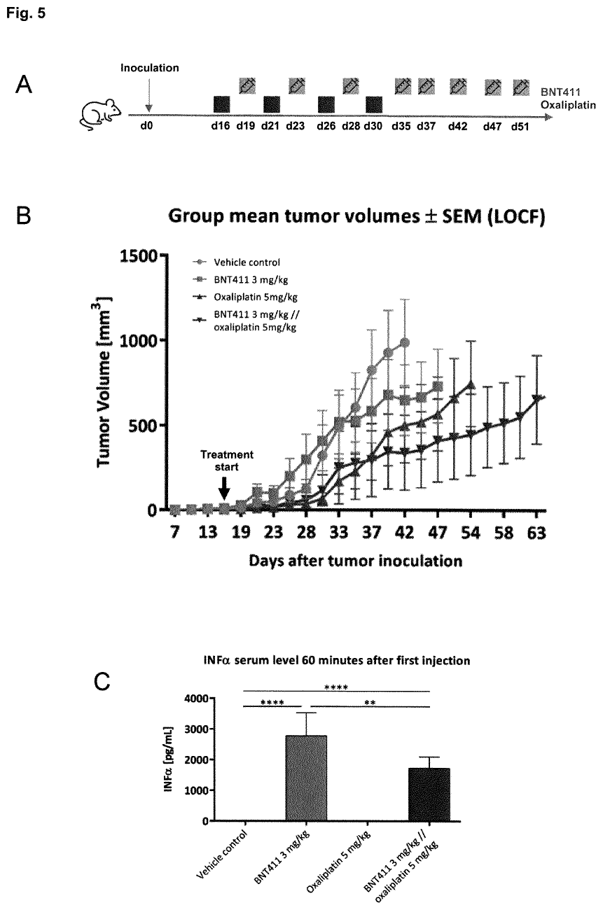

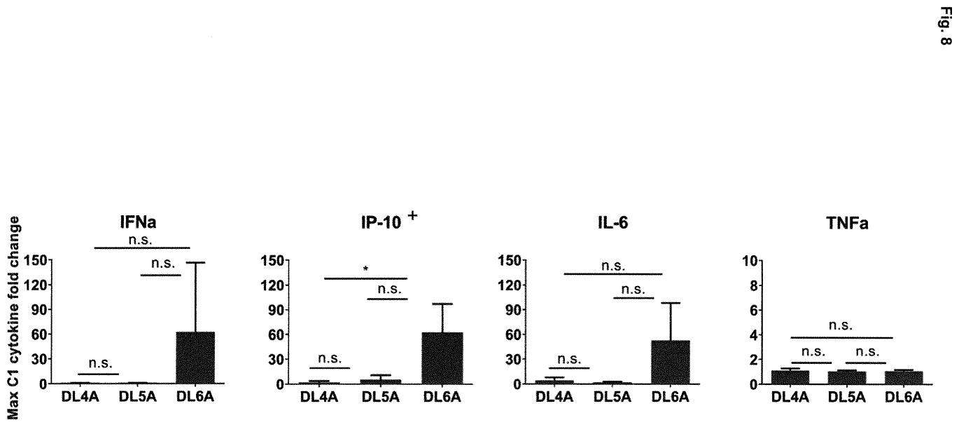

As demonstrated in the present disclosure, upon BNT411 exposure, a higher amount of IFNα was induced at concentrations much lower than the comparative imidazoquinolines imiquimod, resiquimod and 852A; cf., e.g., FIG. 1. For inducing secretion of pyrogenic proinflammatory cytokines (e.g., TNFα) up to two orders of magnitude higher concentrations of BNT411 are required; cf., e.g., FIG. 7. Taken together, this stronger activity and greater selectivity indicates a higher potency and broader therapeutic window for systemically administered BNT411. In vitro and in vivo studies with BNT411 in mouse models demonstrate release of high levels of therapeutically relevant IFNα; cf., e.g., FIG. 2. In addition, the present disclosure demonstrates that in mouse tumor models BNT411 exhibits anti-tumor activity in monotherapy as well as in combination with standard therapies (such as in combination with a platinum-based chemotherapeutic agent or a checkpoint inhibitor); cf., e.g., FIGS. 4 to 6. Moreover, the present inventors show for the first time that BNT411 (i) has an acceptable safety profile at several doses tested in human patients; (ii) induces a substantial type-I IFN-dominated cytokine response in human patients; and (iii) does not lead to a substantial increase of proinflammatory cytokines such as tumor necrosis factor alpha (TNFα), optionally TNFα and IL-6, at least at some therapeutically relevant doses; cf., e.g., Examples 6 and 7 as well as FIG. 8 of the present application.

### Compound BNT411

Compound BNT411 has the following formula:

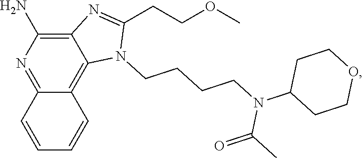 [SMILES: COCCc1nc2c(N)nc3ccccc3c2n1CCCCN(C(C)=O)C1CCOCC1]

or is a pharmaceutically acceptable solvate and/or salt thereof.

In some embodiments, BNT411 is a solvate. In some embodiments, the solvate of BNT411 is selected from the group consisting of a hydrate and a dimethylsulfoxide solvate. In some embodiments, the solvate of BNT411 is a hydrate. In some embodiments, the solvate of BNT411 is a dimethylsulfoxide solvate.

In some embodiments, BNT411 is anhydrous.

BNT411 is a selective TLR7 agonist. Signaling through TLR7 initiates a cascade of effects culminating in a spectrum of innate and adaptive immune mechanisms. In some embodiments, engagement of TLR7 on plasmacytoid dendritic cells (pDCs) results in the recruitment of MyD88, interferon regulatory factor 7 and NF-κB, which induces the secretion of IFNα and proinflammatory cytokines, maturation of antigen-presenting cells and upregulation of costimulatory factors (CD80/CD86) (cf., e.g., FIG. 3 of the present disclosure; Gilliet et al. Nat Rev Immunol. 2008; 8:594-606; Swiecki and Colonna. Nat Rev Immunol. 2015; 15 (8): 471-485). IFNα is considered to be the critical cytokine of TLR7 activation, which via pleiotropic mechanisms (direct mediation of tumor cell death; anti-angiogenesis; inhibition of suppressive regulatory T-cells; activation of immune cells, Th1 response) promotes anti-tumoral effects (Smits et al., Oncologist. 2008; 13 (8): 859-75). Furthermore, the TLR7-induced maturation of pDCs to antigen-presenting cells upregulates processing and (cross) presentation of antigens released from dying tumor cells. Taken together, simultaneous IFNa secretion and antigen (cross) presentation promote the efficient priming and activation of antigen-specific CD4 and CD8 T-cells and expansion of cytotoxic T-lymphocytes (CTLs). These CTLs migrate into the tumor tissue, recognize tumor-specific antigens and lyse tumor cells (Markov et al., Acta Naturae. 2016; 8 (3): 17-30). In addition, TLR7 signaling drives a strong innate immune response. As an example, IFNa secreted by pDCs activates natural killer (NK) cells, which enhances their cytotoxic potential towards major histocompatibility complex (MHC)-deficient targets (Wiedemann et al., Oncoimmunology. 2016; 5 (7): e1189051). Furthermore, a reduction of the suppressive index of the tumor microenvironment is observed since the frequency of polymorphonuclear myeloid-derived suppressor cells (PMN-MDSC) is reduced and repolarization of tumor-associated macrophages is induced (Williams et al., NPJ Breast Cancer. 2016; 2. pii: 15025).

TLR7 ligands of the imidazoquinoline family such as imiquimod, resiquimod or 852A have been extensively studied as immunotherapy drugs. In animal models strong anti-tumoral effects have been observed, however only imiquimod has been approved for human use so far. Repeated i.v. administrations of imiquimod at a systemically effective IFNa-inducing dose level are associated with dose limiting adverse reactions (Witt et al., Cancer Research. 1993; 53, 5176-80; Holldack, Drug Discov Today. 2014; April; 19 (4): 379-82). As a consequence, imiquimod is registered for topical use only, in localized diseases such as basal cell carcinoma, genital warts and bladder cancer (Edwards, J Am Acad Dermatol. 2000; July; 43 (1 Pt 2): S12-7; Spencer, Dermatol Surg. 2006; January; 32 (1): 63-9; Stanley, Clin Exp Dermatol. 2002; October; 27 (7): 571-7; Vacchelli et al., Oncoimmunology. 2012; 1 (6): 894-907). Similarly, repeat-dose regimens of resiquimod and 852A are not well tolerated, limiting the clinical use of these compounds again to the treatment of localized disease (Rook et al., Blood. 2015; 17; 126 (12): 1452-6; Sabado et al., Cancer Immunol Res. 2015; 3 (3): 278-87; Geller et al., Cancer Immunol. Immunother. 2010; 59:1877-84).

The extensive data on strong anti-tumor activity of TLR7 agonists in animal cancer models are in strong contrast to the limited clinical efficacy of systemic TLR7 agonists observed so far. Without being bound to any theory, the reasons for that may be a combination of: i) dose limiting toxicities due to release of a broad spectrum of proinflammatory cytokines, ii) limited IFNa release due to low agonist potency, iii) exhaustion of pDCs due to short dosing intervals, iv) use of the TLR7 agonist in monotherapy whereas a greater efficacy is expected in combination therapy as defined by the underlying mode-of-action.

The unfavorable safety profile of other TLR7 agonists may be attributable to the release of a broader array of proinflammatory cytokines on top of the intended type-I IFN induction (Gorden et al., J Immunol. 2005 Feb. 1; 174 (3): 1259-68; Hamm et al., J Immunotoxicol. 2009; December; 6 (4): 257-65). The induction of a broader range of proinflammatory cytokines beyond IFNa, is a known challenge of TLR7 agonists and is hypothesized to be linked to TLR8 coactivation. To optimize the safety profile and to increase the therapeutic window, BNT411 was developed to selectively bind to TLR7.

As shown in the present application, BNT411 induces primarily IFNa-release at the doses tested. Besides this improved cytokine profile, higher amounts of IFNa are secreted as compared to other TLR7 agonists such as resiquimod. This optimized cytokine profile and increased potency provides a broad therapeutic window and results in a better risk benefit profile as supported by the clinical safety data presented herein; cf., e.g., Example 7.

In some embodiments, the effects of TLR7 engagement by BNT411 result in immune responses that may synergize with and complement current treatment regimens. For example, TLR7-triggered pDC activation may enhance priming and expansion of antigen-specific cytotoxic T lymphocytes (CTLs), once tumor antigens are released through radio/chemotherapy-induced cell death. However, the checkpoint blockade may counteract immunosuppressive signals and avoid exhaustion of CTLs which works together with the changes of the tumor microenvironment (TME) induced by downstream effects of TLR7 activation. As shown in the present application (cf., Examples 3 to 5), BNT411 was tested in a syngenic subcutaneous mouse CT-26 tumor model. BNT411 as single agent was shown to cause tumor growth retardation and increase the frequency of T-cells specific for the gp70 antigen present on CT-26 tumor cells, indicating that adaptive immunity contributes to the anti-tumor effect. Furthermore, combining BNT411 with oxaliplatin had an even more profound effect on tumor growth retardation.

In some embodiments, compound BNT411 is administered in a suitable amount, i.e., the amount of compound BNT411 administered, e.g., in each dose and/or treatment cycle, may induce intracellular signaling when binding to TLR7 expressed on cells, in particular plasmacytoid dendritic cells (pDCs).

In some embodiments, the suitable amount provides one or more of the following:
inducing the secretion of IFNa, optionally IP-10, from cells (in particular, blood cells, especially pDCs);
recruiting MyD88, interferon regulatory factor 7 (IRF7) and/or NF-κB;
inducing a type-I IFN-dominated cytokine profile;
triggering maturation of pDCs into APCs, preferably thereby upregulating processing and (cross) presentation of antigens released from dying tumor cells;
activating natural killer (NK) cells (in particular by IFNa secreted from cells such as pDCs).

In some embodiments, the suitable amount induces the secretion of IFNa from cells (in particular blood cells, especially pDCs). In some embodiments, the amount of IFNa secreted from cells (in particular blood cells, especially pDCs) is increased compared to control cells (in particular, cells not treated by BNT411). In some embodiments, the increase of secreted IFNa is at least 10-fold (such as at least 20-fold, at least 30-fold, at least 40-fold, or at least 50-fold) compared to control cells.

In some embodiments, the suitable amount induces the secretion of IFNa and IP-10 from cells (in particular blood cells, especially pDCs). In some embodiments, the amount of IFNa and IP-10 secreted from cells (in particular blood cells, especially pDCs) is increased compared to control cells (in particular, cells not treated by BNT411). In some embodiments, the increase of secreted IFNa is at least 10-fold (such as at least 20-fold, at least 30-fold, at least 40-fold, or at least 50-fold) compared to control cells and/or the increase of secreted IP-10 is at least 10-fold (such as at least 20-fold, at least 30-fold, at least 40-fold, or at least 50-fold) compared to control cells. In some embodiments, the increase of secreted IFNa and secreted IP-10 is each at least 10-fold (such as at least 20-fold, at least 30-fold, at least 40-fold, or at least 50-fold) compared to control cells.

In some embodiments, the suitable amount induces a type-I IFN-dominated cytokine profile. The expression “type-I IFN-dominated cytokine profile” as used herein means that upon activation (in particular, upon treatment with a TLR7 agonist) the cytokine profile secreted from cells (in particular blood cells, especially pDCs) mainly differs from the cytokine profile secreted from control cells (i.e., non-activated cells, in particular cells not treated with a TLR7 agonist) in that IFNa (and optionally IP-10 or a further type-I IFN cytokine) is present, preferably its amount is increased (at least 10-fold, such as at least 20-fold, at least 30-fold, at least 40-fold, or at least 50-fold). In certain embodiments, the expression “type-I IFN-dominated cytokine profile” as used herein means that upon activation (in particular upon treatment with a TLR7 agonist) the cytokine profile secreted from cells (in particular blood cells, especially pDCs) mainly differs the cytokine profile secreted from control cells (i.e., non-activated cells, in particular cells not treated with a TLR7 agonist) in that IFNa (and optionally IP-10 or a further type-I IFN cytokine) is present, preferably its amount is increased (at least 10-fold, such as at least 20-fold, at least 30-fold, at least 40-fold, or at least 50-fold), and the amount of TNFa remains essentially unchanged (in particular, the amount of secreted TNFa upon activation is at most 2-fold (such as at most 1.5-fold, 1.4-fold, 1.3-fold, 1.2-fold or 1.2-fold) the amount of secreted TNFa without activation) or is decreased.

In some embodiments, the suitable amount results in recruiting MyD88, interferon regulatory factor 7 (IRF7) and/or NF-κB.

In some embodiments, the suitable amount results in maturation of pDCs into APCs. In some embodiments, this maturation upregulates processing and (cross) presentation of antigens released from dying tumor cells.

In some embodiments, by simultaneous IFNα secretion and antigen (cross) presentation, the suitable amount is able to promote the efficient priming and activation of antigen-specific CD4 and CD8 T-cells and expansion of cytotoxic T-lymphocytes (CTLs). In some embodiments, these CTLs migrate into the tumor tissue, recognize tumor-specific antigens and lyse tumor cells.

In some embodiments, the suitable amount results in the activation of natural killer (NK) cells (in particular by IFNa secreted from cells such as pDCs). In certain embodiments, the activation of NK cells drives an innate immune response, which, for example, enhances cytotoxic potential of NK cells towards MHC-deficient targets.

In some embodiments, the suitable amount is safe. In some embodiments, a safe amount is an amount resulting in no adverse events related to the medical treatment or procedure (in particular related to the administration of BNT411). In some embodiments, a safe amount is an amount resulting in adverse events of at most grade 3. In some embodiments, a safe amount is an amount resulting in adverse events of at most grade 2. In some embodiments, a safe amount is an amount resulting in adverse events of at most grade 3 which are related to the medical treatment or procedure (in particular related to the administration of BNT411). In some embodiments, a safe amount is an amount resulting in adverse events of at most grade 2 which are related to the medical treatment or procedure (in particular related to the administration of BNT411).

In some embodiments, the suitable amount of BNT411, e.g., in each dose and/or in each treatment cycle, is
a) about 0.1 μg/kg to about 50 μg/kg body weight or about 8 μg to about 4000 μg in total; and/or
b) about 0.23×10⁻⁹ mol/kg body weight to about 114×10⁻⁹ mol/kg body weight or about 18×10⁻⁹ mol to about 9100×10⁻⁹ mol in total.

In some embodiments, the suitable amount of BNT411, e.g., in each dose and/or in each treatment cycle, is
a) about 0.5 μg/kg to about 20 μg/kg body weight or about 40 μg to about 1600 μg in total; and/or
b) about 1.14×10⁻⁹ mol/kg body weight to about 45.5×10⁻⁹ mol/kg body weight or about 91×10⁻⁹ mol to about 3640×10⁻⁹ mol in total.

In some embodiments, the suitable amount of BNT411, e.g., in each dose and/or in each treatment cycle, is
a) about 2 μg/kg to about 10 μg/kg body weight or about 160 μg to about 800 μg in total; and/or
b) about 4.55×10⁻⁹ mol/kg body weight to about 22.8×10⁻⁹ mol/kg body weight or about 364×10⁻⁹ mol to about 1820×10⁻⁹ mol in total.

In some embodiments, the suitable amount of BNT411, e.g., in each dose and/or in each treatment cycle, is
a) about 2 μg/kg to about 9 μg/kg body weight or about 160 μg to about 720 μg in total; and/or
b) about 4.55×10⁻⁹ mol/kg body weight to about 20.5×10⁻⁹ mol/kg body weight or about 364×10⁻⁹ mol to about 1638×10⁻⁹ mol in total.

In some embodiments, the suitable amount of BNT411, e.g., in each dose and/or in each treatment cycle, is
a) about 2 μg/kg to about 5 μg/kg body weight or about 160 μg to about 400 μg in total; and/or
b) about 4.55×10⁻⁹ mol/kg body weight to about 11.4×10⁻⁹ mol/kg body weight or about 364×10⁻⁹ mol to about 910×10⁻⁹ mol in total.

In some embodiments, the suitable amount of BNT411, e.g., in each dose and/or in each treatment cycle, is
a) about 2.4 μg/kg to about 9.6 μg/kg body weight or about 192 μg to about 768 μg in total; and/or
b) about 5.46×10⁻⁹ mol/kg body weight to about 21.8×10⁻⁹ mol/kg body weight or about 437×10⁻⁹ mol to about 1747×10⁻⁹ mol in total.

In some embodiments, the suitable amount of BNT411, e.g., in each dose and/or in each treatment cycle, is
a) about 4.8 μg/kg to about 9.6 μg/kg body weight or about 384 μg to about 768 μg in total; and/or
b) about 10.9×10⁻⁹ mol/kg body weight to about 21.8×10⁻⁹ mol/kg body weight or about 873×10⁻⁹ mol to about 1747×10⁻⁹ mol in total.

In some embodiments, the suitable amount of BNT411, e.g., in each dose and/or in each treatment cycle, is
a) about 4.8 μg/kg body weight or about 384 μg in total; and/or
b) about 10.9×10⁻⁹ mol/kg body weight or about 874×10⁻⁹ mol in total.

According to these embodiments, the dose defined in μg/kg body weight (or mol/kg body weight) may be converted to flat dose, and vice versa, based on the median body weight of the subjects to whom BNT411 is administered being 80 kg. Furthermore, the dose defined in mol (or mol/kg body weight) may be converted the dose in μg (or μg/kg body weight), and vice versa, based on the molecular weight of BNT411 (i.e., 439.55 g/mol for anhydrous BNT411).

BNT411 may be administered in any manner and by any route known in the art. In some embodiments, BNT411 is administered intravenously, intraarterially, subcutaneously, intradermally, intramuscularly, intranodally, or intratumorally. In some embodiments, BNT411 is administered systemically, such as parenterally, in particular intravenously.

BNT411 may be administered in the form of any suitable pharmaceutical composition as described herein. In some embodiments, BNT411 is administered in the form of an infusion.

In some embodiments, BNT411 is administered in at least one treatment cycle. In some embodiments, each treatment cycle is about two weeks (14 days), three weeks (21 days) or four weeks (28 days). In some embodiments, each treatment cycle is three weeks (21 days).

In some embodiments, one dose of BNT411 is administered (such as infused) once a week (Q1W) for at least 1 treatment cycle. In some embodiments, one dose of BNT411 is administered (such as infused) Q1W for at least 4 treatment cycles and then every 3 weeks (Q3W). In some embodiments, the administration of BNT411 is continued until disease progression, occurrence of unacceptable adverse effects, or death.

In some embodiments, each dose of BNT411 is infused over a minimum of 10 minutes, such as over a minimum of 15 minutes, a minimum of 20 minutes, a minimum of 25 minutes or a minimum of 30 minutes.

In some embodiments, each dose of BNT411 is administered as bolus injection, such as slow bolus injection. In some embodiments, each dose of BNT411 is administered as bolus injection over a minimum of 1 min, such as over a minimum of 2 min, a minimum of 3 min, a minimum of 4 min, or a minimum of 5 min, and/or over a maximum of 9 min, such as over a maximum of 8 min, a maximum of 7 min, a maximum of 6 min, or a maximum of 5 min. In some embodiments, each dose of BNT411 is administered as bolus injection within 1 to 10 min, such as within 3 to 5 min.

### Additional Treatments

In certain embodiments, additional treatments may be administered to a patient in combination with the monotherapy with BNT411 described herein. Such additional treatments include classical cancer therapy, e.g., radiation therapy, surgery, hyperthermia therapy and/or chemotherapy. Furthermore, such additional treatments include treatments involving immune checkpoint modulators.

Chemotherapy is a type of cancer treatment that uses one or more anti-cancer drugs (chemotherapeutic agents), usually as part of a standardized chemotherapy regimen. The term chemotherapy has come to connote non-specific usage of intracellular poisons to inhibit mitosis. The connotation excludes more selective agents that block extracellular signals (signal transduction). The development of therapies with specific molecular or genetic targets, which inhibit growth-promoting signals from classic endocrine hormones (primarily estrogens for breast cancer and androgens for prostate cancer) are now called hormonal therapies. By contrast, other inhibitions of growth-signals like those associated with receptor tyrosine kinases are referred to as targeted therapy.

Importantly, the use of drugs (whether chemotherapy, hormonal therapy or targeted therapy) constitutes systemic therapy for cancer in that they are introduced into the blood stream and are therefore in principle able to address cancer at any anatomic location in the body. Systemic therapy is often used in conjunction with other modalities that constitute local therapy (i.e. treatments whose efficacy is confined to the anatomic area where they are applied) for cancer such as radiation therapy, surgery or hyperthermia therapy.

Traditional chemotherapeutic agents are cytotoxic by means of interfering with cell division (mitosis) but cancer cells vary widely in their susceptibility to these agents. To a large extent, chemotherapy can be thought of as a way to damage or stress cells, which may then lead to cell death if apoptosis is initiated.

In some embodiments, the effects of TLR7 engagement by BNT411 result in immune responses that may synergize with and complement current treatment regimens. For example, TLR7-triggered pDC activation may enhance priming and expansion of antigen-specific cytotoxic T lymphocytes (CTLs), once tumor antigens are released through radio/chemotherapy-induced cell death. However, the checkpoint blockade may counteract immunosuppressive signals and avoid exhaustion of CTLs which works together with the changes of the tumor microenvironment (TME) induced by downstream effects of TLR7 activation. Thus, in some embodiments, in order to avoid such checkpoint blockade, BNT411 therapy is combined with therapy using a checkpoint modulator, in particular a checkpoint inhibitor.

### Additional Therapeutic Agents

Besides the monotherapy, in some embodiments, compound BNT411 is used in combination with one or more additional therapeutic agents. In some embodiments, the one or more additional therapeutic agents comprise those therapeutic agents which are commonly used in the treatment of a tumor or cancer as described herein, such as checkpoint inhibitors (CPIs), chemotherapeutic agents (e.g., platinum-based chemotherapeutic agents and/or topoisomerase inhibitors), and combinations thereof.

### Chemotherapeutic Agents

In some embodiments, the one or more additional therapeutic agents comprise one or more chemotherapeutic agents, in particular, those chemotherapeutic agents which are commonly used in the treatment of a tumor or cancer as described herein.

Chemotherapeutic agents include alkylating agents, antimetabolites, anti-microtubule agents, topoisomerase inhibitors, and cytotoxic antibiotics.

Alkylating agents have the ability to alkylate many molecules, including proteins, RNA and DNA. The subtypes of alkylating agents are the nitrogen mustards, nitrosoureas, tetrazines, aziridines, platinum-based chemotherapeutic compounds, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Platinum-based chemotherapeutic compounds include cisplatin, carboplatin and oxaliplatin. They impair cell function by forming covalent bonds with the amino, carboxyl, sulfhydryl, and phosphate groups in biologically important molecules. Non-classical alkylating agents include procarbazine and hexamethylmelamine.

Anti-metabolites are a group of molecules that impede DNA and RNA synthesis. Many of them have a similar structure to the building blocks of DNA and RNA. Anti-metabolites resemble either nucleobases or nucleosides, but have altered chemical groups. These drugs exert their effect by either blocking the enzymes required for DNA synthesis or becoming incorporated into DNA or RNA. Subtypes of the anti-metabolites are the anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents block cell division by preventing microtubule function. The vinca alkaloids prevent the formation of the microtubules, whereas the taxanes prevent the microtubule disassembly. Vinca alkaloids include vinorelbine, vindesine, and vinflunine. Taxanes include docetaxel (Taxotere) and paclitaxel (Taxol).

Topoisomerase inhibitors are drugs that affect the activity of two enzymes: topoisomerase I and topoisomerase II and include irinotecan, topotecan, camptothecin, etoposide, doxorubicin, mitoxantrone, teniposide, novobiocin, merbarone, and aclarubicin.

The cytotoxic antibiotics are a varied group of drugs that have various mechanisms of action. The common theme that they share in their chemotherapy indication is that they interrupt cell division. The most important subgroup is the anthracyclines (e.g., doxorubicin, daunorubicin, epirubicin, idarubicin pirarubicin, and aclarubicin) and the bleomycins; other prominent examples include mitomycin C, mitoxantrone, and actinomycin.

In some embodiments, e.g., prior to administration of immune effector cells, a lymphodepleting treatment may be applied, e.g., by administering cyclophosphamide and fludarabine. Such treatment may increase cell persistence and the incidence and duration of clinical responses.

In some embodiments, a chemotherapeutic agent (or a combination of two or more chemotherapeutic agents) is administered in a suitable amount. In some embodiments, the amount of chemotherapeutic agent (or the amount of a combination of two or more chemotherapeutic agents) administered, e.g., in each dose and/or treatment cycle, may totally or partially reduce or prevent progression of a tumor or cancer.

The amount of a chemotherapeutic agent administered, e.g., in each dose and/or in each treatment cycle, will depend, inter alia, on the chemotherapeutic agent administered.

In some embodiments, where the chemotherapeutic agent comprises a platinum-based chemotherapeutic agent, the amount of platinum-based chemotherapeutic agent administered, e.g., in each dose and/or in each treatment cycle, is
a) about 100-1000 mg in total; and/or
b) about 0.27×10⁻³−2.7×10⁻³ mol in total.

In some embodiments, where the chemotherapeutic agent comprises a topoisomerase inhibitor (such as etoposide), the amount of topoisomerase inhibitor administered, e.g., in each dose and/or in each treatment cycle, is
a) about 150-400 mg in total; and/or
b) about 0.17×10⁻³−0.68×10⁻³ mol in total.

Chemotherapeutic agents may be administered in any manner and by any route known in the art. The mode and route of administration will depend on the type of chemotherapeutic agent to be used. In some embodiments, in particular those where the chemotherapeutic agent comprises a platinum-based chemotherapeutic agent (such as carboplatin), the platinum-based chemotherapeutic agent is administered systemically, such as parenterally, in particular intravenously. In some embodiments, in particular those where the chemotherapeutic agent comprises a topoisomerase inhibitor (such as etoposide), the topoisomerase inhibitor is administered systemically, such as parenterally, in particular intravenously, or is administered orally. In some embodiments, in particular those where the chemotherapeutic agent comprises a topoisomerase inhibitor (such as etoposide) and is administered more than once per treatment cycle, the administration route may be the same or different. In some embodiments, in particular those where the chemotherapeutic agent comprises a topoisomerase inhibitor (such as etoposide) and is administered more than once per treatment cycle, the first dose of the topoisomerase inhibitor in one treatment cycle is administered systemically, such as parenterally, in particular intravenously, and the second (and each subsequent dose) of the topoisomerase inhibitor in said treatment cycle is administered enterally, in particular orally.

Chemotherapeutic agents may be administered in the form of any suitable pharmaceutical composition as described herein. In some embodiments, in particular those where the chemotherapeutic agent comprises a platinum-based chemotherapeutic agent (such as carboplatin), the platinum-based chemotherapeutic agent is administered in the form of an infusion. In some embodiments, in particular those where the chemotherapeutic agent comprises a platinum-based chemotherapeutic agent (such as carboplatin), the platinum-based chemotherapeutic agent is infused over a minimum of 30 minutes, such as over a minimum of 40 minutes, a minimum of 45 minutes, a minimum of 50 minutes or a minimum of 60 minutes.

In some embodiments, in particular those where the chemotherapeutic agent comprises a topoisomerase inhibitor (such as etoposide), the topoisomerase inhibitor is administered in the form of an infusion and/or capsules (such as soft capsules). In some embodiments, in particular those where the chemotherapeutic agent comprises a topoisomerase inhibitor (such as etoposide) and is administered more than once per treatment cycle, the administration form may be the same or different. In some embodiments, in particular those where the chemotherapeutic agent comprises a topoisomerase inhibitor (such as etoposide) and is administered more than once per treatment cycle, the first dose of the topoisomerase inhibitor in one treatment cycle is administered in the form of an infusion, and the second (and each subsequent dose) of the topoisomerase inhibitor in said treatment cycle is administered in the form of capsules (such as soft capsules). In some embodiments, in particular those where the chemotherapeutic agent comprises a topoisomerase inhibitor (such as etoposide) and is administered in the form of an infusion, the topoisomerase inhibitor may be infused over any period of time, such as at least 5 min (e.g., at least 10 min, at least 15 min, at least 20 min, or at least 30 min) and/or at most 120 min (such as at most 100 min, at most 90 min, at most 80 minutes, at most 70 min, at most 60 min, at most 50 min, or at most 45 min).

In some embodiments, the chemotherapeutic agent is administered at least once for one treatment cycle. In some embodiments, in particular those where the chemotherapeutic agent comprises a platinum-based chemotherapeutic agent (such as carboplatin), the platinum-based chemotherapeutic agent is administered at least once for two or more (such as two to four) treatment cycles. In some embodiments, the treatment cycle has three weeks (21 days). In some embodiments, in particular those where the chemotherapeutic agent comprises a platinum-based chemotherapeutic agent (such as carboplatin), the platinum-based chemotherapeutic agent is administered Q3W (preferably at day 1 of a treatment cycle) for at least two treatment cycles (such as two to four treatment cycles). In some embodiments, in particular those where the chemotherapeutic agent comprises a platinum-based chemotherapeutic agent (such as carboplatin), the platinum-based chemotherapeutic agent is administered Q3W (preferably at day 1 of a treatment cycle) for four treatment cycles.

In some embodiments, in particular those where the chemotherapeutic agent comprises a topoisomerase inhibitor (such as etoposide), the topoisomerase inhibitor is administered at least once for two or more (such as at least three, or at least four) treatment cycles. In some embodiments, the treatment cycle has three weeks (21 days). In some embodiments, in particular those where the chemotherapeutic agent comprises a topoisomerase inhibitor (such as etoposide), the topoisomerase inhibitor is administered three times in the first week of a treatment cycle (preferably at day 1, day 2 and day 3 of a treatment cycle) for two or more (such as at least three, or at least four) treatment cycles. In some embodiments, in particular those where the chemotherapeutic agent comprises a topoisomerase inhibitor (such as etoposide), the topoisomerase inhibitor is administered three times in the first week of a treatment cycle (preferably at day 1, day 2 and day 3 of a treatment cycle) for 4 treatment cycles.

In some embodiments, the one or more chemotherapeutic agents comprise a platinum-based chemotherapeutic agent. In some embodiments, the one or more chemotherapeutic agents comprise cisplatin, oxaliplatin, or carboplatin. In some embodiments, the one or more chemotherapeutic agents comprise oxaliplatin, or carboplatin. In some embodiments, the one or more chemotherapeutic agents comprise carboplatin. In some embodiments, the platinum-based chemotherapeutic agent is administered in an amount (e.g., about 100-1000 mg in total; and/or about 0.27×10⁻³−2.7×10⁻³ mol in total), by a route (e.g., intravenously), in a form (e.g., infusion), within a time period (e.g., over a minimum of 30 minutes), and/or in a dose regimen (e.g., Q3W (preferably at day 1 of a treatment cycle) for four treatment cycles) as specified above.

In some embodiments, the one or more chemotherapeutic agents comprise a topoisomerase inhibitor, such as an inhibitor of topoisomerase I and/or topoisomerase II. In some embodiments, the one or more chemotherapeutic agents comprise a compound selected from the group consisting of irinotecan, topotecan, camptothecin, etoposide, ciprofloxaxin, doxorubicin, mitoxantrone, teniposide, novobiocin, merbarone, and aclarubicin. In some embodiments, the one or more chemotherapeutic agents comprise a compound selected from the group consisting of etoposide, ciprofloxaxin, and doxorubicin. In some embodiments, the one or more chemotherapeutic agents comprise etoposide. In some embodiments, the topoisomerase inhibitor is administered in an amount (e.g., about 150-400 mg in total; and/or about 0.17×10⁻³−0.68×10⁻³ mol in total), by a route (e.g., intravenously or orally), in a form (e.g., infusion or capsules), within a time period (e.g., at least 5 min), and/or in a dose regimen (e.g., three times in the first week of a treatment cycle (preferably at day 1, day 2 and day 3 of a treatment cycle) for 4 treatment cycles) as specified above.

### Checkpoint Inhibitors

In some embodiments, the immune checkpoint inhibitor suitable for use in the methods disclosed herein, is an antagonist of inhibitory signals, e.g., an antibody which targets, for example, PD-1, or PD-L1. These ligands and receptors as well as other checkpoint proteins (such as receptors and their ligands) are described and reviewed in Pardoll, D., Nature. 12:252-264, 2012. Further immune checkpoint proteins that can be targeted according to the disclosure are described herein.

In some embodiments, the immune checkpoint inhibitor prevents inhibitory signals associated with the immune checkpoint. In some embodiments, the immune checkpoint inhibitor is an antibody, or fragment thereof that disrupts or inhibits inhibitory signaling associated with the immune checkpoint. In some embodiments, the immune checkpoint inhibitor is a small molecule inhibitor that disrupts or inhibits inhibitory signaling. In some embodiments, the immune checkpoint inhibitor is a peptide-based inhibitor that disrupts or inhibits inhibitory signaling. In some embodiments, the immune checkpoint inhibitor is an inhibitory nucleic acid molecule that disrupts or inhibits inhibitory signaling.

In some embodiments, inhibiting or blocking of inhibitory immune checkpoint signaling, as described herein, results in preventing or reversing immune-suppression and establishment or enhancement of T cell immunity, in particular against cancer cells. In some embodiments, inhibition of immune checkpoint signaling, as described herein, reduces or inhibits dysfunction of the immune system. In some embodiments, inhibition of immune checkpoint signaling, as described herein, renders dysfunctional immune cells less dysfunctional. In some embodiments, inhibition of immune checkpoint signaling, as described herein, renders a dysfunctional T cell less dysfunctional.

In some embodiments, the immune checkpoint inhibitor prevents the interaction between checkpoint blocker proteins, e.g., the interaction between PD-1 and PD-L1 or PD-L2.

The immune checkpoint inhibitor may be an antibody, an antigen-binding fragment thereof, or a construct thereof comprising an antibody portion with an antigen-binding fragment of the required specificity. Antibodies or antigen-binding fragments thereof are as described herein. Antibodies or antigen-binding fragments thereof that are immune checkpoint inhibitors include in particular antibodies or antigen-binding fragments thereof that bind to immune checkpoint proteins, such as immune checkpoint receptors or immune checkpoint receptor ligands. Antibodies or antigen-binding fragments may also be conjugated to further moieties, as described herein. In particular, antibodies or antigen-binding fragments thereof are chimerized, humanized or human antibodies. Preferably, immune checkpoint inhibitor antibodies or antigen-binding fragments thereof are antagonists of immune checkpoint receptors or of immune checkpoint receptor ligands.

In some preferred embodiments, an antibody that is an immune checkpoint inhibitor is an isolated antibody.

In some embodiments, the immune checkpoint inhibitor is an antibody, a fragment, construct thereof or antibody mimic that prevents the interaction between checkpoint blocker proteins, e.g., an antibody, fragment, construct thereof, or antibody mimic that prevents the interaction between PD-1 and PD-L1 or PD-L2.

The immune checkpoint inhibitor may be an inhibitory nucleic acid molecule, such as an oligonucleotide, siRNA, shRNA, an antisense DNA or RNA molecule, and an aptamer (e.g., DNA or RNA aptamer), in particular an antisense-oligonucleotide. In some embodiments, the immune checkpoint inhibitor being siRNA interferes with mRNA therefore blocking translation, e.g., translation of an immune checkpoint protein, e.g., translation of PD-1, PD-L1, and/or PD-L2.

The checkpoint inhibitor may also be in the form of the soluble form of the molecules (or variants thereof) themselves, e.g., a soluble PD-L1 or PD-L1 fusion.

In the context of the disclosure, more than one checkpoint inhibitor can be used, wherein the more than one checkpoint inhibitors are targeting distinct checkpoint pathways or the same checkpoint pathway. Preferably, the more than one checkpoint inhibitors are distinct checkpoint inhibitors. Preferably, if more than one distinct checkpoint inhibitor is used, in particular at least 2, 3, 4, 5, 6, 7, 8, 9 or 10 distinct checkpoint inhibitors are used, preferably 2, 3, 4 or 5 distinct checkpoint inhibitors are used, more preferably 2, 3 or 4 distinct checkpoint inhibitors are used, even more preferably 2 or 3 distinct checkpoint inhibitors are used and most preferably 2 distinct checkpoint inhibitors are used.

In some embodiments, the immune checkpoint inhibitor is a PD-1 axis binding antagonist. As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In some embodiments, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to one or more of its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In some embodiments, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In some embodiments, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

In some embodiments, the inhibitory immunoregulator (immune checkpoint blocker) is a component of the PD-1/PD-L1 or PD-1/PD-L2 signaling pathway. Accordingly, in some embodiments of the disclosure, the checkpoint inhibitor is an inhibitor of the PD-1 signaling pathway. In certain embodiments, the checkpoint inhibitor of the PD-1 signaling pathway is a PD-1 inhibitor. In certain embodiments, the checkpoint inhibitor of the PD-1 signaling pathway is a PD-1 ligand inhibitor, such as a PD-L1 inhibitor or a PD-L2 inhibitor. In some preferred embodiments, the checkpoint inhibitor of the PD-1 signaling pathway is an antibody, an antigen-binding portion thereof or a construct thereof that disrupts or inhibits the interaction between the PD-1 receptor and one or more of its ligands, PD-L1 and/or PD-L2. Antibodies which bind to PD-1 and disrupt or inhibit the interaction between PD-1 and one or more of its ligands are known in the art. In certain embodiments, the antibody, antigen-binding portion thereof or a construct thereof binds specifically to PD-1. In certain embodiments, the antibody, antigen-binding portion thereof or a construct thereof binds specifically to PD-L1 and disrupts or inhibits its interaction with PD-1, thereby increasing immune activity. In certain embodiments, the antibody, antigen-binding portion thereof or a construct thereof binds specifically to PD-L2 and disrupts or inhibits its interaction with PD-1, thereby increasing immune activity.

In some embodiments, the PD-1 binding antagonist is a molecule that inhibits the binding of PD-1 to its ligand binding partner(s). In a specific aspect, the PD-1 ligand binding partners are PD-L1 and/or PD-L2.

In some embodiments, the PD-L1 binding antagonist is a molecule that inhibits the binding of PD-L1 to its binding partner(s). In a specific aspect, PD-L1 binding partner(s) are PD-1 and/or B7-1.

In some embodiments, the PD-L2 binding antagonist is a molecule that inhibits the binding of PD-L2 to its binding partner(s). In a specific aspect, a PD-L2 binding partner is PD-1.

The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody).

In some embodiments, the PD-L1 binding antagonist is an anti-PD-L1 antibody.

PD-1 axis binding antagonists such as anti-PD-1 antibodies and anti-PD-L1 antibodies may be administered in any manner and by any route known in the art. The mode and route of administration will depend on the type of PD-1 axis binding antagonist to be used.

PD-1 axis binding antagonists may be administered in the form of any suitable pharmaceutical composition as described herein.

PD-1 axis binding antagonists such as anti-PD-1 antibodies and anti-PD-L1 antibodies may be administered in the form of nucleic acid, such DNA or RNA, encoding a PD-1 axis binding antagonist such as anti-PD-1 antibody or anti-PD-L1 antibody. For example, antibodies can be delivered encoded in expressing nucleic acids, as described herein. Nucleic acid molecules can be delivered as such, e.g., in the form of a plasmid or mRNA molecule, or complexed with a delivery vehicle, e.g., a liposome, lipoplex or any other nucleic-acid particle such as nucleic-acid lipid particle. PD-1 axis binding antagonists such as anti-PD-1 antibodies and anti-PD-L1 antibodies may also be administered via an oncolytic virus comprising an expression cassette encoding the PD-1 axis binding antagonist.

Exemplary PD-1 inhibitors include, without limitation, anti-PD-1 antibodies such as BGB-A317 (BeiGene; see U.S. Pat. No. 8,735,553, WO 2015/35606 and US 2015/0079109), lambrolizumab (e.g., disclosed as hPD109A and its humanized derivatives h409A1, h409A16 and h409A17 in WO2008/156712), AB137132 (Abcam), EH12.2H7 and RMP1-14 (#BE0146; Bioxcell Lifesciences Pvt. LTD.), MIH4 (Affymetrix eBioscience), nivolumab (OPDIVO, BMS-936558; Bristol Myers Squibb; see U.S. Pat. No. 8,008,449; WO 2013/173223; WO 2006/121168), pembrolizumab (KEYTRUDA; MK-3475; Merck; see WO 2008/156712), pidilizumab (CT-011; CureTech; see Hardy et al., 1994, Cancer Res., 54 (22): 5793-6 and WO 2009/101611), PDR001 (Novartis; see WO 2015/112900), MEDI0680 (AMP-514; AstraZeneca; see WO 2012/145493), TSR-042 (see WO 2014/179664), cemiplimab (REGN-2810; Regeneron; H4H7798N; cf. US 2015/0203579 and WO 2015/112800), JS001 (TAIZHOU JUNSHI PHARMA; see Si-Yang Liu et al., 2007, J. Hematol. Oncol. 70:136), AMP-224 (GSK-2661380; cf. Li et al., 2016, Int J Mol Sci 17 (7): 1151 and WO 2010/027827 and WO 2011/066342), PF-06801591 (Pfizer), tislelizumab (BGB-A317; BeiGene; see WO 2015/35606, U.S. Pat. No. 9,834,606, and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANB011; see WO2014/179664), GLS-010 (Wuxi/Harbin Gloria Pharmaceuticals; also known as WBP3055; see Si-Yang et al., 2017, J. Hematol. Oncol. 70:136), STI-1110 (Sorrento Therapeutics; see WO 2014/194302), AGEN2034 (Agenus; see WO 2017/040790), MGA012 (Macrogenics; see WO 2017/19846), IBI308 (Innovent; see WO 2017/024465, WO 2017/025016, WO 2017/132825, and WO 2017/133540), cetrelimab (JNJ-63723283; JNJ-3283; see Calvo et al., J. Clin. Oncol. 36, no. 5_suppl (2018) 58), genolimzumab (CBT-501; see Patel et al., J. ImmunoTher. Cancer, 2017, 5 (Suppl 2): P242), sasanlimab (PF-06801591; see Youssef et al., Proc. Am. Assoc. Cancer Res. Ann. Meeting 2017; Cancer Res 2017; 77 (13 Suppl): Abstract), toripalimab (JS-001; see US 2016/0272708), camrelizumab (SHR-1210; INCSHR-1210; see US 2016/376367; Huang et al., Clin. Cancer Res. 2018; 24 (6): 1296-1304), spartalizumab (PDR001; see WO 2017/106656; Naing et al., J. Clin. Oncol. 34, no. 15_suppl (2016) 3060-3060), BCD-100 (JSC BIOCAD, Russia; see WO 2018/103017), balstilimab (AGEN2034; see WO 2017/040790), sintilimab (IBI-308; see WO 2017/024465 and WO 2017/133540), ezabenlimab (BI-754091; see US 2017/334995; Johnson et al., J. Clin. Oncol. 36, no. 5_suppl (2018) 212-212), zimberelimab (GLS-010; see WO 2017/025051), LZM-009 (see US 2017/210806), AK-103 (see WO 2017/071625, WO 2017/166804, and WO 2018/036472), retifanlimab (MGA-012; see WO 2017/019846), Sym-021 (see WO 2017/055547), CS1003 (see CN107840887), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105, 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28 (9): 665-678 and Sasikumar and Ramachandra, 2018, BioDrugs, 32 (5): 481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831.

In a certain embodiment, the PD-1 inhibitor is nivolumab (OPDIVO; BMS-936558), pembrolizumab (KEYTRUDA; MK-3475), pidilizumab (CT-011), PDR001, MEDI0680 (AMP-514), TSR-042, REGN2810, JS001, AMP-224 (GSK-2661380), PF-06801591, BGB-A317, BI 754091, or SHR-1210.

In certain embodiments, the inhibitory immunoregulator is an anti-PD-1 antibody or antigen-binding fragment thereof comprising the complementary determining regions (CDRs) of one of the anti-PD-1 antibodies or antigen-binding fragments described above, such as the CDRs of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In some embodiments, the CDRs of the anti-PD-1 antibody are delineated using the Kabat numbering scheme (Kabat, E. A., et al. (1991) Sequences of Proteins of Immunological Interest, Fifth Edition, U.S. Department of Health and Human Services, NTH Publication No. 91-3242).

In certain embodiments, the inhibitory immunoregulator is an anti-PD-1 antibody or antigen-binding fragment thereof comprising the heavy chain variable region and the light chain variable region of one of the anti-PD-1 antibodies or antigen-binding fragments described above, such as the heavy chain variable region and the light chain variable region of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In certain embodiments, the inhibitory immunoregulator is an anti-PD-1 antibody or antigen-binding fragment thereof selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

Anti-PD-1 antibodies of the disclosure are preferably monoclonal, and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein binds specifically to PD-1 (e.g., human PD-1).

The immunoglobulin molecules of the disclosure can be of any isotype (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-PD-1 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken.

The anti-PD-1 antibodies disclosed herein may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

The anti-PD-1 antibodies disclosed herein may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003; 27 (1): 55-77 (“IMGT” numbering scheme); Honegger A and Plückthun A, “Yet another numbering scheme for immunoglobulin variable domains: an automatic modeling and analysis tool,” J Mol Biol, 2001; 309 (3): 657-70, (“Aho” numbering scheme); and Martin et al., “Modeling antibody hypervariable loops: a combined algorithm,” PNAS, 1989, 86 (23): 9268-9272, (“AbM” numbering scheme). The boundaries of a given CDR may vary depending on the scheme used for identification. In some embodiments, a CDR or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-1 antibodies or antigen-binding fragments thereof provided herein are according to the IMGT numbering scheme as described in Lefranc, M. P. et al., Dev. Comp. Immunol., 2003, 27, 55-77.

In some embodiments, the anti-PD-1 antibodies disclosed herein comprise the CDRs of the antibody nivolumab. See WO 2006/121168. In some embodiments, the CDRs of the antibody nivolumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-1 antibody or derivative thereof comprising a heavy or light chain variable domain, said variable domain comprising (a) a set of three CDRs, in which said set of CDRs are from the monoclonal antibody nivolumab, and (b) a set of four framework regions, in which said set of framework regions differs from the set of framework regions in the monoclonal antibody nivolumab, and in which said anti-PD-1 antibody or derivative thereof binds to PD-1. In certain embodiments, the anti-PD-1 antibody is nivolumab.

Anti-PD-1 antibodies disclosed herein may also be described or specified in terms of their binding affinity to PD-1 (e.g., human PD-1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The anti-PD-1 antibodies also include derivatives and constructs that are modified, i.e., by the covalent attachment of any type of molecule to the antibody such that covalent attachment does not prevent the antibody from binding to PD-1. For example, but not by way of limitation, the anti-PD-1 antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative or construct may contain one or more non-classical amino acids.

Exemplary PD-1 ligand inhibitors are PD-L1 inhibitors and PD-L2 inhibitors and include, without limitation, anti-PD-L1 antibodies such as MEDI4736 (durvalumab; AstraZeneca; see WO 2011/066389), MSB-0010718C (see US 2014/0341917), YW243.55.S70 (see SEQ ID NO: 20 of WO 2010/077634 and U.S. Pat. No. 8,217,149), MIH1 (Affymetrix eBioscience; cf. EP 3 230 319), MDX-1105 (Roche/Genentech; see WO2013019906 and U.S. Pat. No. 8,217,149) STI-1014 (Sorrento; see WO2013/181634), CK-301 (Checkpoint Therapeutics), KN035 (3D Med/Alphamab; see Zhang et al., 2017, Cell Discov. 3:17004), atezolizumab (TECENTRIQ; RG7446; MPDL3280A; R05541267; see U.S. Pat. No. 9,724,413), BMS-936559 (Bristol Myers Squibb; see U.S. Pat. No. 7,943,743, WO 2013/173223), avelumab (bavencio; cf. US 2014/0341917), LY3300054 (Eli Lilly Co.), CX-072 (Proclaim-CX-072; also called CytomX; see WO2016/149201), FAZ053, KN035 (see WO2017020801 and WO2017020802), MDX-1105 (see US 2015/0320859), anti-PD-L1 antibodies disclosed in U.S. Pat. No. 7,943,743, including 3G10, 12A4 (also referred to as BMS-936559), 10A5, 5F8, 10H10, 1B12, 7H1, 11E6, 12B7, and 13G4, anti-PD-L1 antibodies as described in WO 2010/077634, U.S. Pat. No. 8,217,149, WO 2010/036959, WO 2010/077634, WO 2011/066342, U.S. Pat. Nos. 8,217,149, 7,943,743, WO 2010/089411, U.S. Pat. Nos. 7,635,757, 8,217,149, US 2009/0317368, WO 2011/066389, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO 2018/222711, WO2015/112805, WO2015/061668, WO2014/159562, WO2014/165082, WO2014/100079.

In some embodiments, the one or more chemotherapeutic agents comprise atezolizumab.

According to the disclosure, the immune checkpoint inhibitor is an inhibitor of an inhibitory checkpoint protein but preferably not an inhibitor of a stimulatory checkpoint protein.

In some preferred embodiments, the immune checkpoint inhibitor is an antibody, in particular an antagonistic or blocking antibody, which disrupts or inhibits one of the inhibitory immune checkpoint signaling pathways described herein. In some preferred embodiments, the immune checkpoint inhibitor is an antibody, in particular an antagonistic or blocking antibody, which disrupts or inhibits the PD-1 pathway (interaction of PD-1 with one or more of its ligands (such as PD-L1 and/or PD-L2)). In some preferred embodiments, the immune checkpoint inhibitor is an antibody, in particular an antagonistic or blocking antibody, which disrupts or inhibits the interaction between PD-1 and PD-L1.

Checkpoint inhibitors may be administered in the form of nucleic acid, such DNA or RNA molecules, encoding an immune checkpoint inhibitor, e.g., an inhibitory nucleic acid molecule or an antibody or fragment thereof. For example, antibodies can be delivered encoded in expression vectors, as described herein. Nucleic acid molecules can be delivered as such, e.g., in the form of a plasmid or mRNA molecule, or complexed with a delivery vehicle, e.g., a liposome, lipoplex or nucleic-acid lipid particles. Checkpoint inhibitors may also be administered via an oncolytic virus comprising an expression cassette encoding the checkpoint inhibitor. Checkpoint inhibitors may also be administered by administration of endogeneic or allogeneic cells able to express a checkpoint inhibitor, e.g., in the form of a cell based therapy.

In some embodiments, the cell based therapy comprises genetically engineered cells. In some embodiments, the genetically engineered cells express an immune checkpoint inhibitor, such as described herein. In some embodiments, the genetically engineered cells express an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. Genetically engineered cells may also express further agents that enhance T cell function. Such agents are known in the art. Cell based therapies for the use in inhibition of immune checkpoint signaling are disclosed, e.g., in WO 2018/222711, herein incorporated by reference in its entirety.

In some embodiments, the checkpoint inhibitor is administered in a suitable amount. In some embodiments, the amount of checkpoint inhibitor administered, e.g., in each dose and/or treatment cycle, may totally or partially reduce, inhibit, interfere with or negatively modulate one or more checkpoint proteins or may totally or partially reduce, inhibit, interfere with or negatively modulate expression of one or more checkpoint proteins. Thus, a checkpoint inhibitor in a suitable amount according to the present disclosure is able to totally or partially reduce, inhibit, interfere with or negatively modulate one or more checkpoint proteins or is able to totally or partially reduce, inhibit, interfere with or negatively modulate expression of one or more checkpoint proteins. Therefore, in some embodiments, the checkpoint inhibitors prevent inhibitory signals associated with the immune checkpoint resulting in preventing or reversing immune-suppression and establishment or enhancement of T cell immunity against cancer cells.

In some embodiments, the amount of checkpoint inhibitor administered in each dose and/or treatment cycle may in particular be in a range, wherein more than 5%, preferably more than 10%, more preferably more than 15%, even more preferably more than 20%, even more preferably more than 25%, even more preferably more than 30%, even more preferably more than 35%, even more preferably more than 40%, even more preferably more than 45%, most preferably more than 50% of said checkpoint inhibitors bind to the checkpoint protein.

In some embodiments, the amount of checkpoint inhibitor administered, e.g., in each dose and/or in each treatment cycle, is
a) about 100-2000 mg in total; and/or
b) about 0.20×10⁻⁹−10×10⁻⁶ mol in total.

In some embodiments, where the checkpoint inhibitor is an antibody (such as an anti-PD-L1 antibody, e.g., atezolizumab), the amount of checkpoint inhibitor administered, e.g., in each dose and/or in each treatment cycle, is
a) about 1000-1400 mg in total; and/or
b) about 6.8×10⁻⁶−9.7×10⁻⁶ mol in total.

Checkpoint inhibitors may be administered in any manner and by any route known in the art. The mode and route of administration will depend on the type of checkpoint inhibitor to be used. In some embodiments, in particular those where the checkpoint inhibitor is an antibody (such as an anti-PD-L1 antibody, e.g., atezolizumab), the checkpoint inhibitor is administered systemically, such as parenterally, in particular intravenously.

Checkpoint inhibitors may be administered in the form of any suitable pharmaceutical composition as described herein. In some embodiments, in particular those where the checkpoint inhibitor is an antibody (such as an anti-PD-L1 antibody, e.g., atezolizumab), the checkpoint inhibitor is administered in the form of an infusion.

In some embodiments, in particular those where the checkpoint inhibitor is an antibody (such as an anti-PD-L1 antibody, e.g., atezolizumab), the checkpoint inhibitor is infused over a minimum of 30 minutes, such as over a minimum of 40 minutes, a minimum of 45 minutes, a minimum of 50 minutes or a minimum of 60 minutes.

In some embodiments, in particular those where the checkpoint inhibitor is an antibody (such as an anti-PD-L1 antibody, e.g., atezolizumab), the checkpoint inhibitor is administered at least once per treatment cycle. In some embodiments, the treatment cycle has three weeks (21 days). In some embodiments, in particular those where the checkpoint inhibitor is an antibody (such as an anti-PD-L1 antibody, e.g., atezolizumab), the checkpoint inhibitor is administered Q3W (preferably at day 1 of a treatment cycle) for at least 2 treatment cycles. In some embodiments, in particular those where the checkpoint inhibitor is an antibody (such as atezolizumab), the checkpoint inhibitor is administered Q3W (preferably at day 1 of a treatment cycle) for each treatment cycle.

### Combinations of Additional Therapeutic Agents

Besides BNT411, the treatment regimen according to the present disclosure (e.g., according to the first aspect, the third aspect and/or the fifth aspect of the present disclosure) may further comprise administering to the subject two or more additional therapeutic agents.

In some embodiments, the treatment regimen according to the first aspect of the present disclosure may further comprise administering to the subject two or more additional therapeutic agents selected from the group consisting of checkpoint inhibitors (CPIs), platinum-based chemotherapeutic agents, and topoisomerase inhibitors.

In some embodiments, the treatment regimen according to the first aspect of the present disclosure may further comprise administering to the subject (i) a checkpoint inhibitor and a platinum-based chemotherapeutic agent; (ii) a checkpoint inhibitor and a topoisomerase inhibitor; or (iii) a checkpoint inhibitor, a platinum-based chemotherapeutic agent, and a topoisomerase inhibitor.

In some embodiments, the treatment regimen according to the first aspect of the present disclosure may further comprise administering to the subject an anti-PD-L1 antibody, a platinum-based chemotherapeutic agent (e.g., carboplatin), and a topoisomerase II inhibitor (e.g., etoposide).

In some embodiments, the treatment regimen according to the first aspect of the present disclosure may further comprise administering to the subject two or more additional therapeutic agents selected from the group consisting of atezolizumab, carboplatin, and etoposide.

In some embodiments, the treatment regimen according to the first aspect of the present disclosure may further comprise administering to the subject atezolizumab, carboplatin, and etoposide.

In some embodiments, the two or more additional therapeutic agents are administered as disclosed above (e.g., with respect to administration amount, route, form, time period and/or dose regimen). For example, the platinum-based chemotherapeutic agent being part of a combination therapy with BNT411 and at least one additional therapeutic agent (such as a checkpoint inhibitor, a topoisomerase II inhibitor, or a combination of a checkpoint inhibitor and a topoisomerase II inhibitor) may administered in an amount as specified above (e.g., about 100-1000 mg in total; and/or about 0.27×10⁻³−2.7×10⁻³ mol in total), by a route as specified above (e.g., intravenously), in a form as specified above (e.g., infusion), within a time period as specified above (e.g., over a minimum of 30 minutes), and/or in a dose regimen as specified above (e.g., Q3W (preferably at day 1 of a treatment cycle) for four treatment cycles).

Additionally, or alternatively, in some embodiments, the checkpoint inhibitor being part of a combination therapy with BNT411 and at least one additional therapeutic agent (such as a platinum-based chemotherapeutic agent, a topoisomerase II inhibitor, or a combination of a platinum-based chemotherapeutic agent and a topoisomerase II inhibitor) may be administered in an amount as specified above (e.g., about 150-400 mg in total; and/or about 0.17×10⁻³−0.68×10⁻³ mol in total), by a route as specified above (e.g., intravenously or orally), in a form as specified above (e.g., infusion or capsules), within a time period as specified above (e.g., at least 5 min), and/or in a dose regimen as specified above (e.g., three times in the first week of a treatment cycle (preferably at day 1 (e.g., i.v.), day 2 (e.g., orally) and day 3 (e.g., orally) of a treatment cycle) for 4 treatment cycles).

In some embodiments, where BNT411 is administered in combination with one, two or more additional therapeutic agents, BNT411 is administered as disclosed above (e.g., with respect to administration amount, route, form, time period and/or dose regimen). Thus, in some embodiments, where BNT411 is administered in combination with one, two or more additional therapeutic agents, BNT411 is administered in an amount as specified above (e.g., about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total), by a route as specified above (e.g., intravenously), in a form as specified above (e.g., infusion), within a time period as specified above (e.g., over a minimum of 15 minutes), and/or in a dose regimen as specified above (e.g., Q1W for at least 4 treatment cycles and then every 3 weeks (Q3W)). In some embodiments, BNT411 is administered Q1W at day 2, day 8, and day 15 of a treatment cycle for 4 treatment cycles and then Q3W at day 2 of treatment cycle 5 and each subsequent treatment cycle.

### Subject and Tumor or Cancer to be Treated

The subject to be treated according to the present disclosure is preferably a human subject.

The tumor or cancer to be treated may be any tumor or cancer. Examples of tumors/cancers include, but are not limited to, leukemias, seminomas, melanomas, sarcomas, myelomas (such as multiple myeloma), teratomas, lymphomas (such as Hodgkin's lymphoma, non-Hodgkin's lymphoma), mesotheliomas, neuroblastomas, gliomas, myelodysplastic syndromes, rectal cancer, endometrial cancer, ureteral cancer, kidney cancer, renal cancer, urothelial cancer, bladder cancer, esophageal cancer, adrenal cancer, adrenocortical cancer, thyroid cancer, blood cancer, skin cancer (such as Merkel cell carcinoma), cancer of the brain, cervical cancer, malignant solitary fibrous tumor, intestinal cancer, liver cancer, thymoma and thymic carcinoma, colon cancer, stomach cancer, intestine cancer, head and neck cancer, gastric cancer, gastrointestinal cancer, lymph node cancer, esophagus cancer, colorectal cancer, pancreas cancer, ear, nose and throat (ENT) cancer, breast cancer, prostate cancer, penile cancer, cancer of the uterus, ovarian cancer and lung cancer (e.g., non-small cell lung cancer (NSCLC) or small cell lung cancer (SCLC)) and the metastases thereof.

In some embodiments, the tumor or cancer to be treated is a solid tumor or cancer.

In some embodiments, the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

In some embodiments, the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

In some embodiments, the subject is chemotherapy-naïve. Thus, in some embodiments, the subject has not received prior chemotherapy, i.e., before the treatment according to the present disclosure (in particular, according to the first aspect, the third aspect, and/or the fifth aspect), the subject has not received any chemotherapeutic agents.

The tumor or cancer may in particular be a lung cancer. The lung cancer may be a non-small cell lung cancer (NSCLC), such as a squamous or a non-squamous NSCLC, or may be a small cell lung cancer (SCLC), such as ES-SCLC. Lung cancer is the second most common malignancy with an estimated age-standardized incidence rate of 22.4 per 100,000 and a leading cause of cancer death for both men and women (Kantar, 2021, SEER, 2018). Worldwide, approximately 2,206,771 new cases of lung cancer and 1,796,144 deaths are estimated in 2020 (GLOBOCAN, 2020). Non-small-cell lung cancer (NSCLC) accounts for 85% to 90% of all cases, with a 5-year survival rate of approximately 18% across all stages of the disease, and only 3.5% for metastatic disease (Jemal et al., 2011) (Kantar, 2021). In the 1L setting, treatment typically consists of platinum-based chemotherapy in combination with immunotherapy, or a targeted therapy, depending on molecular and biomarker analysis and the histology of the tumor (NCCN, 2021d). More recently, the advent of PD-1 and programmed death ligand 1 (PD-L1) inhibitors have improved outcomes for patients without driver mutations (approximately 62% of the non-squamous population and 77% of the squamous population (Kantar, 2021)). More treatment alternatives are needed for patients whose tumors do not harbor certain oncogenic mutations or do not express the biomarker for checkpoint inhibitor (CPI) options. Novel combinations with complementary approaches to enhance response may further address the unmet need in this population. For patients in the second line (2L) setting, standard-of-care chemotherapy (SOC) is limited to platinum-based chemotherapy, a CPI monotherapy or docetaxel with or without ramucirumab depending on the previous therapy received. For patients in the third-line (3L) setting, chemotherapy monotherapy is the standard. Novel therapies are needed to limit toxicity and potentially enhance efficacy in this population (NCCN, 2021d).

In some embodiments, the tumor or cancer is a small cell lung cancer (SCLC), such as extensive-stage small cell lung cancer (ES-SCLC).

### Treatment Regimen

BNT411 and, if present or used, the one or more additional therapeutic agents can be administered as specified above, in particular with respect to the administration amount, route, form, time period and/or dose regimen.

In some embodiments, BNT411 is administered in an amount of about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total. In some embodiments, BNT411 is administered in an amount of about 2.4 μg/kg to about 9.6 μg/kg body weight (such as about 4.8 μg/kg to about 9.6 μg/kg body weight) or about 192 μg to about 768 μg (such as about 384 μg to about 768 μg) in total; and/or about 5.46×10⁻⁹ mol/kg body weight to about 21.8×10⁻⁹ mol/kg body weight (such as about 10.9×10⁻⁹ mol/kg body weight to about 21.8×10⁻⁹ mol/kg body weight) or about 437×10⁻⁹ mol to about 1747×10⁻⁹ mol (such as about 873×10⁻⁹ mol to about 1747×10⁻⁹ mol) in total. In some embodiments, BNT411 is administered in an amount of about 4.8 μg/kg body weight or about 384 μg in total; and/or about 10.9×10⁻⁹ mol/kg body weight or about 874×10⁻⁹ mol in total.

In some embodiments, BNT411 is administered by any suitable way, such as intravenously (e.g., by infusion).

In some embodiments, the one or more additional therapeutic agents, if present or used, are administered by any suitable way, such as intravenously (e.g., by infusion), intraarterially, subcutaneously, intradermally, intramuscularly, intranodally, intratumorally, or orally (e.g., in the form of capsules, such as soft capsules). In some embodiments, where the one or more additional therapeutic agents comprise a checkpoint inhibitor and a platinum-based chemotherapeutic agent, the checkpoint inhibitor and the platinum-based chemotherapeutic agent are administered systemically, in particular intravenously, (e.g., by infusion). In some embodiments, where the one or more additional therapeutic agents comprise a topoisomerase inhibitor, the topoisomerase inhibitor is administered systemically, in particular intravenously, (e.g., by infusion), or enterally, in particular orally (e.g., by capsules). In some embodiments, where the one or more additional therapeutic agents comprise a topoisomerase inhibitor, the topoisomerase inhibitor is administered at least two times (such as at least three times) for at least one treatment cycle (such as for at least 2, 3, or 4 treatment cycles). In certain embodiments, the first dose of the topoisomerase inhibitor of one treatment cycle is administered systemically, in particular intravenously, (e.g., by infusion). In some embodiments, the second dose (and, if present or used, any subsequent dose) of the topoisomerase inhibitor of said treatment cycle is administered orally (e.g., by capsules).

In some embodiments, BNT411 and one or more additional therapeutic agents are administered to the subject in at least one treatment cycle, in particular as specified above with respect to the administration amount, route, form, time period and/or dose regimen.

In some embodiments, the treatment cycle is about two weeks (14 days), three weeks (21 days) or four weeks (28 days). In some embodiments, each treatment cycle is three weeks (21 days).

In some embodiments, BNT411 and, if present or used, the one or more additional therapeutic agents are administered simultaneously. In some embodiments, BNT411 and, if present or used, the one or more additional therapeutic agents the checkpoint inhibitor are administered separately.

In some embodiments, BNT411 is administered Q1W for at least two (such as at least three or at least four) treatment cycles. In some embodiments, BNT411 is administered Q1W at day 2, day 8, and day 15 of a treatment cycle. In some embodiments, each treatment cycle is three weeks (21 days). In some embodiments, BNT411 is administered Q1W for at least two (such as at least three or at least four) treatment cycles and then every 3 weeks (Q3W), in particular at day 2. In some embodiments, BNT411 is administered Q1W at day 2, day 8, and day 15 of a treatment cycle for 4 treatment cycles and then Q3W at day 2 of treatment cycle 5 and each subsequent treatment cycle.

In some embodiments, where the one or more additional therapeutic agents comprise a checkpoint inhibitor (such as an anti-PD-L1 antibody, e.g., atezolizumab), the checkpoint inhibitor is administered at least once per treatment cycle. In some embodiments, each treatment cycle is three weeks (21 days). In some embodiments, the checkpoint inhibitor is administered Q3W (preferably at day 1 of a treatment cycle) for at least 2 treatment cycles. In some embodiments, the checkpoint inhibitor is administered Q3W (preferably at day 1 of a treatment cycle) for each treatment cycle.

In some embodiments, where the one or more additional therapeutic agents comprise a platinum-based chemotherapeutic agent (such as carboplatin), the platinum-based chemotherapeutic agent is administered at least once for two or more (such as two to four) treatment cycles. In some embodiments, each treatment cycle is three weeks (21 days). In some embodiments, the platinum-based chemotherapeutic agent is administered Q3W (preferably at day 1 of a treatment cycle) for at least two treatment cycles (such as two to four treatment cycles). In some embodiments, the platinum-based chemotherapeutic agent is administered Q3W (preferably at day 1 of a treatment cycle) for four treatment cycles (in particular, treatment cycles 1, 2, 3, and 4 (or the initial four treatment cycles)).

In some embodiments, where the one or more additional therapeutic agents comprise a topoisomerase inhibitor (such as etoposide), the topoisomerase inhibitor is administered at least once for two or more (such as at least three, or at least four) treatment cycles. In some embodiments, each treatment cycle is three weeks (21 days). In some embodiments, the topoisomerase inhibitor is administered three times in the first week of a treatment cycle (preferably at day 1, day 2 and day 3 of a treatment cycle) for two or more (such as at least three, or at least four) treatment cycles. In some embodiments, the topoisomerase inhibitor is administered three times in the first week of a treatment cycle (preferably at day 1, day 2 and day 3 of a treatment cycle) for 4 treatment cycles (in particular, treatment cycles 1, 2, 3, and 4 (or the initial four treatment cycles)).

In some embodiments, where the one or more additional therapeutic agents comprise a checkpoint inhibitor (such as an anti-PD-L1 antibody, e.g., atezolizumab), a platinum-based chemotherapeutic agent (such as carboplatin), and a topoisomerase inhibitor (such as etoposide), the checkpoint inhibitor is administered Q3W (preferably at day 1 of a treatment cycle) for each treatment cycle; the platinum-based chemotherapeutic agent is administered Q3W (preferably at day 1 of a treatment cycle) for four treatment cycles (in particular, treatment cycles 1, 2, 3, and 4 (or the initial four treatment cycles)); and the topoisomerase inhibitor is administered three times in the first week of a treatment cycle (preferably at day 1, day 2 and day 3 of a treatment cycle) for 4 treatment cycles (in particular, treatment cycles 1, 2, 3, and 4 (or the initial four treatment cycles)). In some embodiments, BNT411 is administered Q1W at day 2, day 8, and day 15 of a treatment cycle for 4 treatment cycles and then Q3W at day 2 of treatment cycle 5 and each subsequent treatment cycle. In some embodiments, each treatment cycle is three weeks (21 days).

BNT411 and, if present or used, the one or more additional therapeutic agents may be administered in any suitable form (e.g., naked as such). However, in some embodiments, BNT411 and, if present or used, the one or more additional therapeutic agents are administered in the form of any suitable pharmaceutical composition as described herein. In some embodiments, at least BNT411 is administered in the form of a separate pharmaceutical composition. In some embodiments, where one or more additional therapeutic agents are used, BNT411 and at least one (such as at least two or at least three) of the one or more additional therapeutic agents are administered in the form of separate pharmaceutical compositions. For example, in some embodiments, where the one or more additional therapeutic agents comprise a platinum-based chemotherapeutic agent, the platinum-based chemotherapeutic agent is administered in the form of a separate pharmaceutical composition. In some embodiments, where one or more additional therapeutic agents are used, BNT411 and any of the one or more additional therapeutic agents are administered in the form of separate pharmaceutical compositions (i.e., one pharmaceutical composition for BNT411 and one pharmaceutical composition for each of the one or more additional therapeutic agents).

### Compositions

A composition or pharmaceutical composition may be formulated with a carrier, excipient and/or diluent as well as any other components suitable for pharmaceutical compositions, including known adjuvants, in accordance with conventional techniques such as those disclosed in Remington: The Science and Practice of Pharmacy, 19th Edition, Gennaro, Ed., Mack Publishing Co., Easton, PA, 1995. The pharmaceutically acceptable carriers or diluents as well as any known adjuvants and excipients should be suitable for BNT411 and/or, if present or used, the one or more additional therapeutic agents, and the chosen mode of administration. In some embodiments, suitability for carriers and other components of pharmaceutical compositions is determined based on the lack of significant negative impact on the desired biological properties of the chosen compound or pharmaceutical composition (e.g., less than a substantial impact [10% or less relative inhibition, 5% or less relative inhibition, etc.]).

In a second aspect, the present disclosure provides a composition, in particular a pharmaceutical composition, comprising BNT411 in an amount of between about 8 μg to 4000 μg or about 18×10⁻⁹ mol to about 9100×10⁻⁹ mol. In some embodiments, a composition, in particular a pharmaceutical composition, comprising BNT411 comprises BNT411 in an amount of about 40 μg to about 1600 μg or about 91×10⁻⁹ mol to about 3640×10⁻⁹ mol. In some embodiments, a composition, in particular a pharmaceutical composition, comprising BNT411 comprises BNT411 in an amount of about 160 μg to about 800 μg or about 364×10⁻⁹ mol to about 1820×10⁻⁹ mol. In some embodiments, a composition, in particular a pharmaceutical composition, comprising BNT411 comprises BNT411 in an amount of about 160 μg to about 720 μg or about 364×10⁻⁹ mol to about 1638×10⁻⁹ mol. In some embodiments, a composition, in particular a pharmaceutical composition, comprising BNT411 comprises BNT411 in an amount of about 192 μg to about 768 μg (such as about 384 μg to about 768 μg) or about 437×10⁻⁹ mol to about 1747×10⁻⁹ mol (such as about 873×10⁻⁹ mol to about 1747×10⁻⁹ mol). In some embodiments, a composition, in particular a pharmaceutical composition, comprising BNT411 comprises BNT411 in an amount of 384 μg or about 874×10⁻⁹ mol.

In some embodiments, a composition, in particular a pharmaceutical composition, comprising BNT411 is for systemic administration. In some embodiments, the composition is for injection or infusion, such as intravenous injection or infusion. In some embodiments, BNT411 is in aqueous solution, such as in 0.9% NaCl (saline), at a volume of 1-500 ml, such as 5-250 ml. In some embodiments, the composition is a dosage unit form.

In some embodiments, a composition, in particular a pharmaceutical composition, comprising BNT411 is for use in a method for reducing or preventing progression of a tumor or treating cancer in a subject, in particular for use in any method for reducing or preventing progression of a tumor or treating cancer in a subject as described herein. Thus, in a third aspect, the present disclosure provides a composition, in particular a pharmaceutical composition, of the second aspect for use in a method for reducing or preventing progression of a tumor or treating cancer in a subject.

The embodiments disclosed herein with respect to the first aspect (in particular regarding BNT411, its manner of administration (in particular with respect to the administration amount, route, form, time period and/or dose regimen), the optional one or more additional therapeutic agents (in particular with respect to the administration amount, route, form, time period and/or dose regimen), the tumor/cancer to be treated, and/or the subject to be treated) also apply to the composition of the second aspect and/or the composition for use of the third aspect.

Thus, in some embodiments of the second and/or third aspect, the composition is administered to the subject in an amount so as to provide about 0.1 μg/kg to about 50 μg/kg body weight of the compound or about 8 μg to about 4000 μg of the compound in total; and/or about 0.23×10⁻⁹ mol/kg body weight to about 114×10⁻⁹ mol/kg body weight of the compound or about 18×10⁻⁹ mol to about 9100×10⁻⁹ mol of the compound in total.

In some embodiments of the second and/or third aspect, the composition is administered to the subject in an amount so as to provide about 0.5 μg/kg to about 20 μg/kg body weight of the compound or about 40 μg to about 1600 μg of the compound in total; and/or about 1.14×10⁻⁹ mol/kg body weight to about 45.5×10⁻⁹ mol/kg body weight of the compound or about 91×10⁻⁹ mol to about 3640×10⁻⁹ mol of the compound in total.

In some embodiments of the second and/or third aspect, the composition is administered to the subject in an amount so as to provide about 2 μg/kg to about 10 μg/kg body weight of the compound or about 160 μg to about 800 μg of the compound in total; and/or about 4.55×10⁻⁹ mol/kg body weight to about 22.8×10⁻⁹ mol/kg body weight of the compound or about 364×10⁻⁹ mol to about 1820×10⁻⁹ mol of the compound in total.

In some embodiments of the second and/or third aspect, the composition is administered to the subject in an amount so as to provide about 2 μg/kg to about 9 μg/kg body weight of the compound or about 160 μg to about 720 μg of the compound in total; and/or about 4.55×10⁻⁹ mol/kg body weight to about 20.5×10⁻⁹ mol/kg body weight of the compound or about 364×10⁻⁹ mol to about 1638×10⁻⁹ mol of the compound in total.

In some embodiments of the second and/or third aspect, the composition is administered to the subject in an amount so as to provide about 2 μg/kg to about 5 μg/kg body weight of the compound or about 160 μg to about 400 μg of the compound in total; and/or about 4.55×10⁻⁹ mol/kg body weight to about 11.4×10⁻⁹ mol/kg body weight of the compound or about 364×10⁻⁹ mol to about 910×10⁻⁹ mol of the compound in total.

In some embodiments of the second and/or third aspect, the composition is administered to the subject in an amount so as to provide about 2.4 μg/kg to about 9.6 μg/kg body weight of the compound or about 192 μg to about 768 μg of the compound in total; and/or about 5.46×10⁻⁹ mol/kg body weight to about 21.8×10⁻⁹ mol/kg body weight of the compound or about 437×10⁻⁹ mol to about 1747×10⁻⁹ mol of the compound in total.

In some embodiments of the second and/or third aspect, the composition is administered to the subject in an amount so as to provide about 4.8 μg/kg to about 9.6 μg/kg body weight of the compound or about 384 μg to about 768 μg of the compound in total; and/or about 10.9×10⁻⁹ mol/kg body weight to about 21.8×10⁻⁹ mol/kg body weight of the compound or about 873×10⁻⁹ mol to about 1747×10⁻⁹ mol of the compound in total.

In some embodiments of the second and/or third aspect, the composition is administered to the subject in an amount so as to provide about 4.8 μg/kg body weight of the compound or about 384 μg of the compound in total; and/or about 10.9×10⁻⁹ mol/kg body weight of the compound or about 874×10⁻⁹ mol of the compound in total.

A composition, in particular the pharmaceutical composition of BNT411 and, if present, the at least one pharmaceutical composition of the one or more additional therapeutic agents, may include diluents, fillers, salts, buffers, detergents (e.g., a nonionic detergent, such as Tween-20 or Tween-80), stabilizers (e.g., sugars or protein-free amino acids), preservatives, solubilizers, and/or other materials suitable for inclusion in a pharmaceutical composition.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

Pharmaceutically acceptable carriers include any and all suitable solvents, dispersion media, coatings, antibacterial and antifungal agents, isotonicity agents, antioxidants and absorption-delaying agents, and the like that are physiologically compatible with the active compound, in particular BNT411 and/or, if present or used, the one or more additional therapeutic agents as used herein.

Examples of suitable aqueous and non-aqueous carriers which may be employed in the (pharmaceutical) compositions include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the (pharmaceutical) compositions is contemplated.

The term “excipient” as used herein refers to a substance which may be present in a (pharmaceutical) composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

A (pharmaceutical) composition may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

A (pharmaceutical) composition may also comprise isotonicity agents, such as sugars, polyalcohols, such as mannitol, sorbitol, glycerol or sodium chloride in the composition.

A (pharmaceutical) composition may also contain one or more adjuvants appropriate for the chosen route of administration such as preservatives, wetting agents, emulsifying agents, dispersing agents, preservatives or buffers, which may enhance the shelf life or effectiveness of the composition. The composition as used herein may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

“Pharmaceutically acceptable salts” comprise, for example, acid addition salts which may, for example, be formed by using a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, suitable pharmaceutically acceptable salts may include alkali metal salts (e.g., sodium or potassium salts); alkaline earth metal salts (e.g., calcium or magnesium salts); ammonium (NH₄⁻); and salts formed with suitable organic ligands (e.g., quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, arginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, galactate, galacturonate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isobutyrate, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, phthalate, picrate, pivalate, polygalacturonate, propionate, salicylate, stearate, sulfate, suberate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

Salts which are not pharmaceutically acceptable may be used for preparing pharmaceutically acceptable salts and are included in the present disclosure.

In some embodiments, BNT411 and, if used, the one or more additional therapeutic agents may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except in so far as any conventional media or agent is incompatible with the active compound, use thereof in the compositions is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

### Kits

In a fourth aspect, the present disclosure provides a kit comprising (i) BNT411, and (ii) one or more additional therapeutic agents selected from the group consisting of checkpoint inhibitors (CPIs), platinum-based chemotherapeutic agents, topoisomerase inhibitors, and combinations thereof.

In some embodiments, the one or more additional therapeutic agents in the kit of the fourth aspect are selected from the group consisting of atezolizumab, carboplatin, etoposide, and combinations thereof.

In some embodiments, BNT411 and the one or more additional therapeutic agents are for systemic administration, in particular for injection or infusion, such as intravenous injection or infusion.

In some embodiments, the kit of the fourth aspect is for use in a method for reducing or preventing progression of a tumor or treating cancer in a subject, in particular for use in any method for reducing or preventing progression of a tumor or treating cancer in a subject as described herein. Thus, in a fifth aspect, the present disclosure provides a kit of the fourth aspect for use in a method for reducing or preventing progression of a tumor or treating cancer in a subject.

The embodiments disclosed herein with respect to the first aspect (in particular regarding BNT411, its manner of administration (in particular with respect to the administration amount, route, form, time period and/or dose regimen), the optional one or more additional therapeutic agents (in particular with respect to the administration amount, route, form, time period and/or dose regimen), the tumor/cancer to be treated, and/or the subject to be treated) also apply to the kit of the fourth aspect and/or the kit for use of the fifth aspect.

In some embodiments, the kit comprises at least two containers, wherein one thereof contains BNT411 (as such or in the form of a (pharmaceutical) composition) and the second container contains the one or more additional therapeutic agents (as such or in the form of a (pharmaceutical) composition). If the kit comprises more than one additional therapeutic agent, it is preferred that the kit comprises at least three containers, one containing BNT411 (as such or in the form of a (pharmaceutical) composition), one containing the first additional therapeutic agent (as such or in the form of a (pharmaceutical) composition), and at least a third container containing the third additional therapeutic agent (as such or in the form of (a) (pharmaceutical) composition(s)). If the kit comprises at least three additional therapeutic agents, it is preferred that the kit comprises at least four containers, one containing BNT411 (as such or in the form of a (pharmaceutical) composition), one containing the first additional therapeutic agent (as such or in the form of a (pharmaceutical) composition), one containing the second additional therapeutic agent (as such or in the form of a (pharmaceutical) composition), and least a fourth container containing the third additional therapeutic agent (as such or in the form of (a) (pharmaceutical) composition(s)).

The embodiments disclosed herein with respect to any one of the first, second and third aspect (in particular regarding BNT411, its manner of administration (in particular with respect to the administration amount, route, form, time period and/or dose regimen), the one or more additional therapeutic agents (in particular with respect to the administration amount, route, form, time period and/or dose regimen), the tumor/cancer to be treated, and/or the subject to be treated) also apply to the kit of the fourth aspect and/or the kit for use of the fifth aspect.

### Further Aspects

In further aspects, the present disclosure provides methods for reducing or preventing progression of a tumor or treating cancer in a subject using the compound BNT411 as described herein, e.g., using the composition described herein and/or the kit described herein. In some embodiments, such further aspects include (i) a method for reducing or preventing progression of a tumor or treating cancer in a subject, said method comprising administering to said subject BNT411 as specified in the first aspect of the present disclosure; (ii) a method for reducing or preventing progression of a tumor or treating cancer in a subject, said method comprising administering to said subject a composition as specified in the second or third aspect of the present disclosure; and (iii) a method for reducing or preventing progression of a tumor or treating cancer in a subject, said method comprising administering to said subject BNT411 and one or more additional therapeutic agents contained in a kit as specified in the fourth or fifth aspect of the present disclosure.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Further itemized embodiments are as follows:

1. A compound having the following formula

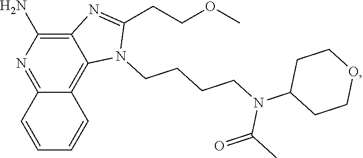 [SMILES: COCCc1nc2c(N)nc3ccccc3c2n1CCCCN(C(C)=O)C1CCOCC1]

or a pharmaceutically acceptable solvate or salt thereof, for use in a method for reducing or preventing progression of a tumor or treating cancer in a subject, said method comprising administering to said subject the compound in a suitable amount.

2. The compound for use of item 1, wherein the suitable amount of the compound is a therapeutically effective and safe amount.

3. The compound for use of any one of the preceding items, wherein the suitable amount of the compound is about 0.1 μg/kg to about 50 μg/kg body weight or about 8 μg to about 4000 μg in total; and/or about 0.23×10⁻⁹ mol/kg body weight to about 114×10⁻⁹ mol/kg body weight or about 18×10⁻⁹ mol to about 9100×10⁻⁹ mol in total.

4. The compound for use of any one of the preceding items, wherein the suitable amount of the compound is about 0.5 μg/kg to about 20 μg/kg body weight or about 40 μg to about 1600 μg in total; and/or about 1.14×10⁻⁹ mol/kg body weight to about 45.5×10⁻⁹ mol/kg body weight or about 91×10⁻⁹ mol to about 3640×10⁻⁹ mol in total.

5. The compound for use of any one of the preceding items, wherein the suitable amount of the compound is about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total.

5a. The compound for use of any one of items 1 to 5, wherein the suitable amount of the compound is about 2.4 μg/kg to about 9.6 μg/kg body weight or about 192 μg to about 768 μg in total; and/or about 5.46×10⁻⁹ mol/kg body weight to about 21.8×10⁻⁹ mol/kg body weight or about 437×10⁻⁹ mol to about 1747×10⁻⁹ mol in total.

5b. The compound for use of any one of items 1 to 5, wherein the suitable amount of the compound is about 4.8 μg/kg to about 9.6 μg/kg body weight or about 384 μg to about 768 μg in total; and/or about 10.9×10⁻⁹ mol/kg body weight to about 21.8×10⁻⁹ mol/kg body weight or about 873×10⁻⁹ mol to about 1747×10⁻⁹ mol in total.

5c. The compound for use of any one of items 1 to 5, wherein the suitable amount of the compound is about 4.8 μg/kg body weight or about 384 μg in total; and/or about 10.9×10⁻⁹ mol/kg body weight or about 874×10⁻⁹ mol in total.

6. The compound for use of any one of the preceding items, wherein the compound is a solvate.

7. The compound for use of item 6, wherein the solvate is selected from the group consisting of a hydrate and a dimethylsulfoxide solvate.

8. The compound for use of any one of items 1 to 5c, wherein the compound is anhydrous.

9. The compound for use of any one of the preceding items, wherein the compound is administered systemically, preferably intravenously.

10. The compound for use of any one of the preceding items, wherein the subject is a human subject.

11. The compound for use of any one of the preceding items, wherein the tumor or cancer is a solid tumor or cancer.

12. The compound for use of any one of the preceding items, wherein the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

13. The compound for use of any one of the preceding items, wherein the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

14. The compound for use of any one of the preceding items, wherein the tumor or cancer is a small cell lung cancer (SCLC), such as extensive-stage small cell lung cancer (ES-SCLC).

15. The compound for use of any one of the preceding items, wherein the subject is chemotherapy-naïve.

16. The compound for use of any one of the preceding items, wherein the compound is administered in at least one treatment cycle, each treatment cycle being three weeks (21 days).

17. The compound for use of any one of the preceding items, wherein one dose of the compound is administered once a week (Q1W) for at least 1 treatment cycle.

18. The compound for use of any one of the preceding items, wherein one dose of the compound is administered Q1W for at least 4 treatment cycles and then every 3 weeks (Q3W).

19. The compound for use of any one of the preceding items, wherein each dose of the compound is infused over a minimum of 10 minutes, such as over a minimum of 15 minutes, a minimum of 20 minutes, a minimum of 25 minutes or a minimum of 30 minutes.

20. The compound for use of any one of the preceding items, wherein the method further comprises administering to said subject one or more additional therapeutic agents.

21. The compound for use of item 20, wherein the one or more additional therapeutic agents are selected from the group consisting of checkpoint inhibitors (CPIs), platinum-based chemotherapeutic agents, topoisomerase inhibitors, and combinations thereof.

22. The compound for use of item 20 or 21, wherein the one or more additional therapeutic agents are selected from the group consisting of atezolizumab, carboplatin, etoposide, and combinations thereof.

23. The compound for use of any one of items 20 to 22, wherein the one or more additional therapeutic agents are a combination of atezolizumab, carboplatin, and etoposide.

24. The compound for use of any one of items 20 to 23, wherein the one or more additional therapeutic agents are administered in at least one treatment cycle, each treatment cycle being three weeks (21 days).

25. The compound for use of any one of items 20 to 24, wherein one dose of each of the one or more additional therapeutic agents is administered at least Q3W for at least 1 treatment cycle, each treatment cycle being three weeks (21 days).

26. The compound for use of any one of items 20 to 25, wherein the one or more additional therapeutic agents are a combination of atezolizumab, carboplatin, and etoposide; one dose of atezolizumab is administered Q3W (preferably at day 1 of a treatment cycle); one dose of carboplatin is administered Q3W (preferably at day 1 of a treatment cycle) for 4 treatment cycles; and one dose of etoposide is administered three times in the first week of a treatment cycle (preferably at day 1, day 2 and day 3 of a treatment cycle) for 4 treatment cycles, each treatment cycle being three weeks (21 days).

27. The compound for use of item 26, wherein one dose of the compound is administered Q1W (preferably at day 2, day 8, and day 15 of a treatment cycle) for 4 treatment cycles and then Q3W (preferably at day 2 of treatment cycle 5 and each subsequent treatment cycle).

28. A composition comprising a compound having the following formula

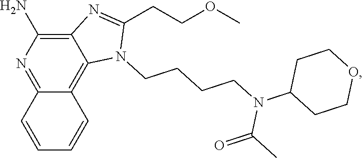 [SMILES: COCCc1nc2c(N)nc3ccccc3c2n1CCCCN(C(C)=O)C1CCOCC1]

or a pharmaceutically acceptable solvate or salt thereof, wherein the amount of the compound in the composition is between about 8 μg to 4000 μg or about 18×10⁻⁹ mol to about 9100×10⁻⁹ mol.

29. The composition of item 28, comprising from about 40 μg to about 1600 μg or about 91×10⁻⁹ mol to about 3640×10⁻⁹ mol of said compound, preferably from about 160 μg to about 800 μg or about 364×10⁻⁹ mol to about 1820×10⁻⁹ mol of said compound.

29a. The composition of item 28 or 29, comprising from about 192 μg to about 768 μg or about 437×10⁻⁹ mol to about 1747×10⁻⁹ mol of said compound.

29b. The composition of item 28 or 29, comprising from about 384 μg to about 768 μg or about 873×10⁻⁹ mol to about 1747×10⁻⁹ mol of said compound.

29c. The composition of item 28 or 29, comprising about 384 μg or about 874×10⁻⁹ mol of said compound.

30. The composition of any one of items 28 to 29c, wherein the composition is for systemic administration.

31. The composition of any one of items 28 to 30, wherein the composition is for injection or infusion, such as intravenous injection or infusion.

32. The composition of any one of items 28 to 31, wherein the compound is in aqueous solution, such as in 0.9% NaCl (saline), at a volume of 1-500 ml, such as 5-250 ml.

33. The composition of any one of items 28 to 32, said composition being a dosage unit form.

34. The composition of any one of items 28 to 33 for use in a method for reducing or preventing progression of a tumor or treating cancer in a subject.

35. The composition for use of item 34, wherein the composition is administered to the subject in an amount so as to provide about 0.1 μg/kg to about 50 μg/kg body weight of the compound or about 8 μg to about 4000 μg of the compound in total; and/or about 0.23×10⁻⁹ mol/kg body weight to about 114×10⁻⁹ mol/kg body weight of the compound or about 18×10⁻⁹ mol to about 9100×10⁻⁹ mol of the compound in total.

36. The compound for use of item 34 or 35, wherein the composition is administered to the subject in an amount so as to provide about 0.5 μg/kg to about 20 μg/kg body weight of the compound or about 40 μg to about 1600 μg of the compound in total; and/or about 1.14×10⁻⁹ mol/kg body weight to about 45.5×10⁻⁹ mol/kg body weight of the compound or about 91×10⁻⁹ mol to about 3640×10⁻⁹ mol of the compound in total.

37. The compound for use of any one of items 34 to 36, wherein the composition is administered to the subject in an amount so as to provide about 2 μg/kg to about 10 μg/kg body weight of the compound or about 160 μg to about 800 μg of the compound in total; and/or about 4.55×10⁻⁹ mol/kg body weight to about 22.8×10⁻⁹ mol/kg body weight of the compound or about 364×10⁻⁹ mol to about 1820×10⁻⁹ mol of the compound in total.

37a. The compound for use of any one of items 34 to 37, wherein the composition is administered to the subject in an amount so as to provide about 2.4 μg/kg to about 9.6 μg/kg body weight of the compound or about 192 μg to about 768 μg of the compound in total; and/or about 5.46×10⁻⁹ mol/kg body weight to about 21.8×10⁻⁹ mol/kg body weight of the compound or about 437×10⁻⁹ mol to about 1747×10⁻⁹ mol of the compound in total.

37b. The compound for use of any one of items 34 to 37, wherein the composition is administered to the subject in an amount so as to provide about 4.8 μg/kg to about 9.6 μg/kg body weight of the compound or about 384 μg to about 768 μg of the compound in total; and/or about 10.9×10⁻⁹ mol/kg body weight to about 21.8×10⁻⁹ mol/kg body weight of the compound or about 873×10⁻⁹ mol to about 1747×10⁻⁹ mol of the compound in total.

37c. The compound for use of any one of items 34 to 37, wherein the composition is administered to the subject in an amount so as to provide about 4.8 μg/kg body weight of the compound or about 384 μg of the compound in total; and/or about 10.9×10⁻⁹ mol/kg body weight of the compound or about 874×10⁻⁹ mol of the compound in total.

38. The composition for use of any one of items 34 to 37c, wherein the subject is a human subject.

39. The composition for use of any one of items 34 to 38, wherein the tumor or cancer is a solid tumor or cancer.

40. The composition for use of any one of items 34 to 39, wherein the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

41. The composition for use of any one of items 34 to 40, wherein the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

42. The composition for use of any one of items 34 to 41, wherein the tumor or cancer is a small cell lung cancer (SCLC), such as extensive-stage small cell lung cancer (ES-SCLC).

43. The composition for use of any one of items 34 to 42, wherein the subject is chemotherapy-naïve.

44. The composition for use of any one of items 34 to 43, wherein the composition is administered in at least one treatment cycle, each treatment cycle being three weeks (21 days).

45. The composition for use of any one of items 34 to 44, wherein one dose of the composition is administered once a week (Q1W) for at least 1 treatment cycle.

46. The composition for use of any one of items 34 to 45, wherein one dose of the composition is administered Q1W for at least 4 treatment cycles and then every 3 weeks (Q3W).

47. The composition for use of any one of items 34 to 46, wherein the composition is infused over a minimum of 10 minutes, such as over a minimum of 15 minutes, a minimum of 20 minutes, a minimum of 25 minutes or a minimum of 30 minutes.

48. The composition for use of any one of items 34 to 47, wherein the method further comprises administering to said subject one or more additional therapeutic agents.

49. The composition for use of item 48, wherein the one or more additional therapeutic agents are selected from the group consisting of checkpoint inhibitors (CPIs), platinum-based chemotherapeutic agents, topoisomerase inhibitors, and combinations thereof.

50. The composition for use of item 48 or 49, wherein the one or more additional therapeutic agents are selected from the group consisting of atezolizumab, carboplatin, etoposide, and combinations thereof.

51. The composition for use of any one of items 48 to 50, wherein the one or more additional therapeutic agents are a combination of atezolizumab, carboplatin, and etoposide.

52. The composition for use of any one of items 48 to 51, wherein the one or more additional therapeutic agents are administered in at least one treatment cycle, each treatment cycle being three weeks (21 days).

53. The composition for use of any one of items 48 to 52, wherein one dose of each of the one or more additional therapeutic agents is administered at least Q3W for at least 1 treatment cycle, each treatment cycle being three weeks (21 days).

54. The composition for use of any one of items 48 to 53, wherein the one or more additional therapeutic agents are a combination of atezolizumab, carboplatin, and etoposide; one dose of atezolizumab is administered Q3W (preferably at day 1 of a treatment cycle); one dose of carboplatin is administered Q3W (preferably at day 1 of a treatment cycle) for 4 treatment cycles; and one dose of etoposide is administered three times in the first week of a treatment cycle (preferably at day 1, day 2 and day 3 of a treatment cycle) for 4 treatment cycles, each treatment cycle being three weeks (21 days).

55. The composition for use of item 54, wherein one dose of the composition is administered Q1W (preferably at day 2, day 8, and day 15 of a treatment cycle) for 4 treatment cycles and then Q3W (preferably at day 2 of treatment cycle 5 and each subsequent treatment cycle).

56. A kit comprising (i) a compound having the following formula

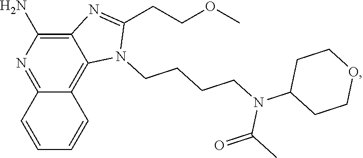 [SMILES: COCCc1nc2c(N)nc3ccccc3c2n1CCCCN(C(C)=O)C1CCOCC1]

or a pharmaceutically acceptable solvate or salt thereof, and (ii) one or more additional therapeutic agents selected from the group consisting of checkpoint inhibitors (CPIs), platinum-based chemotherapeutic agents, topoisomerase inhibitors, and combinations thereof.

57. The kit of item 56, wherein the one or more additional therapeutic agents are selected from the group consisting of atezolizumab, carboplatin, etoposide, and combinations thereof.

58. The kit of item 56 or 57, wherein the compound and the one or more additional therapeutic agents are for systemic administration, in particular for injection or infusion, such as intravenous injection or infusion.

59. The kit of any one of items 56 to 58 for use in a method for reducing or preventing progression of a tumor or treating cancer in a subject.

60. The kit for use of item 59, wherein the compound is administered to the subject in an amount of about 0.1 μg/kg to about 50 μg/kg body weight or about 8 μg to about 4000 μg in total; and/or about 0.23×10⁻⁹ mol/kg body weight to about 114×10⁻⁹ mol/kg body weight or about 18×10⁻⁹ mol to about 9100×10⁻⁹ mol in total.

61. The kit for use of item 59 or 60, wherein the compound is administered to the subject in an amount of about 0.5 g/kg to about 20 μg/kg body weight or about 40 μg to about 1600 μg in total; and/or about 1.14×10⁻⁹ mol/kg body weight to about 45.5×10⁻⁹ mol/kg body weight or about 91×10⁻⁹ mol to about 3640×10⁻⁹ mol in total.

62. The kit for use of any one of items 59 to 61, wherein the compound is administered to the subject in an amount of about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total.

62a. The kit for use of any one of items 59 to 62, wherein the compound is administered to the subject in an amount of about 2.4 μg/kg to about 9.6 μg/kg body weight or about 192 μg to about 768 μg in total; and/or about 5.46×10⁻⁹ mol/kg body weight to about 21.8×10⁻⁹ mol/kg body weight or about 437×10⁻⁹ mol to about 1747×10⁻⁹ mol in total.

62b. The kit for use of any one of items 59 to 62, wherein the compound is administered to the subject in an amount of about 4.8 μg/kg to about 9.6 μg/kg body weight or about 384 μg to about 768 μg in total; and/or about 10.9×10⁻⁹ mol/kg body weight to about 21.8×10⁻⁹ mol/kg body weight or about 873×10⁻⁹ mol to about 1747×10⁻⁹ mol in total.

62c. The kit for use of any one of items 59 to 62, wherein the compound is administered to the subject in an amount of about 4.8 μg/kg body weight or about 384 μg in total; and/or about 10.9×10⁻⁹ mol/kg body weight or about 874×10⁻⁹ mol in total.

63. The kit for use of any one of items 59 to 62c, wherein the subject is a human subject.

64. The kit for use of any one of items 59 to 63, wherein the tumor or cancer is a solid tumor or cancer.

65. The kit for use of any one of items 59 to 64, wherein the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

66. The kit for use of any one of items 59 to 65, wherein the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

67. The kit for use of any one of items 59 to 66, wherein the tumor or cancer is a small cell lung cancer (SCLC), such as extensive-stage small cell lung cancer (ES-SCLC).

68. The kit for use of any one of items 59 to 67, wherein the subject is chemotherapy-naïve.

69. The kit for use of any one of items 59 to 68, wherein the compound is administered in at least one treatment cycle, each treatment cycle being three weeks (21 days).

70. The kit for use of any one of items 59 to 69, wherein one dose of the compound is administered once a week (Q1W) for at least 1 treatment cycle.

71. The kit for use of any one of items 59 to 70, wherein one dose of the compound is administered Q1W for at least 4 treatment cycles and then every 3 weeks (Q3W).

72. The kit for use of any one of items 59 to 71, wherein each dose of the compound is infused over a minimum of 10 minutes, such as over a minimum of 15 minutes, a minimum of 20 minutes, a minimum of 25 minutes or a minimum of 30 minutes.

73. The kit for use of any one of items 59 to 72, wherein the one or more additional therapeutic agents are administered in at least one treatment cycle, each treatment cycle being three weeks (21 days).

74. The kit for use of any one of items 59 to 73, wherein one dose of each of the one or more additional therapeutic agents is administered at least Q3W for at least 1 treatment cycle, each treatment cycle being three weeks (21 days).

75. The kit for use of any one of items 59 to 74, wherein the one or more additional therapeutic agents are a combination of atezolizumab, carboplatin, and etoposide; one dose of atezolizumab is administered Q3W (preferably at day 1 of a treatment cycle); one dose of carboplatin is administered Q3W (preferably at day 1 of a treatment cycle) for 4 treatment cycles; and one dose of etoposide is administered three times in the first week of a treatment cycle (preferably at day 1, day 2 and day 3 of a treatment cycle) for 4 treatment cycles, each treatment cycle being three weeks (21 days).

76. The kit for use of item 75, wherein one dose of the compound is administered Q1W (preferably at day 2, day 8, and day 15 of a treatment cycle) for 4 treatment cycles and then Q3W (preferably at day 2 of treatment cycle 5 and each subsequent treatment cycle).

Further aspects of the present disclosure are disclosed herein.

### EXAMPLES

### Methods

### Cytometric Bead Array (CBA)

After performing a serial dilution of the test compounds (such as BNT411), 5 μL of each sample is transferred/pipetted to single wells of a 96-well-plate. As negative control, plasma which has been prepared from whole blood of each donor is used. Subsequently, 245 μL of RPMI-medium is added to each well, leading to a 1:50 dilution of each sample, named spiking solutions. The heparinized whole blood is pipetted to separate 96-well-plate. After seeding whole blood, the prepared spiking solutions are added: 190 μL of whole blood is seeded in each well and 10 μL of spiking solutions is added to get a final volume of 200 μL per well and additional 1:20 dilution of all samples. Finally, the plates are incubated at 37° C. and 5% CO₂. After 24 h incubation the plates are centrifuged at 500×g for 5 min. The plasma of all samples is harvested, transferred to a new 96-well-plate and frozen at −15° C. to −25° C. until performing the CBA, but at least 3 h.

The CBA is performed with the frozen/thawed plasma samples according to the manufacturer's instructions for ProCarta multiplex kits. For evaluation of cytokine concentrations, assay duplicates of all samples are measured by using the luminex system. After checking standard curves with the instrument control system (Bio-Plex Manager 4.1.1) the raw data are transferred to excel. Graphical analysis is performed with GraphPad Prism 6.

### IFNa ELISA

Serum levels of IFNα were measured by sandwich ELISA (Pbl Assay Systems) according to manufacturer's instructions.

### CT26 Tumor Model

BALB/c mice were inoculated s.c. with 2.5×10⁵−5×10⁵ CT26-WT into the flank, respectively. Once tumors reached palpable sizes, mice were randomly divided into treatment and control groups. Tumor volume was measured unblinded with a caliper and calculated using the formula (a×b²)/2 (a being the largest and b being the smallest diameter of the tumor). Tumor growth was documented as mean tumor size with standard error disregarding single distant outliers. Treatment was initiated after 7-10 d with tumors having reached an average volume of about 10 mm³. 3 mg/kg (75 μg/mouse; BALB/c mice) BNT411 was administered by i.v. injection to the retro-orbital venous plexus. Control groups received an equal volume of 100 μl saline vehicle (0.1% 1 M HCl, 0.1% 1 M NaOH, 99.8% isotonic NaCl solution pH 5.63).

### Example 1

Human PBMCs (peripheral blood mononuclear cells) were obtained from healthy donors and treated in vitro with BNT411 (concentration range: 0.1 nM to 10 μM) or a comparative imidazoquinoline (resiquimod (R848; concentration range: 0.6 nM to 50 μM); 852A (concentration range: 0.6 nM to 50 μM); or imiquimod (R837; concentration range: 0.6 nM to 50 μM). 24 h after stimulation secretion of several cytokines including IFNα was analyzed by a multiplexed CBA. The results of the CBA measurement are shown in FIG. 1 (data shown as mean±standard deviation (SD), n=5).

As can be seen from FIG. 1, upon BNT411 exposure, a significantly higher amount of IFNα was induced at lower concentrations (about 10 to 30-fold lower concentrations) compared to the imidazoquinolines imiquimod (R837), resiquimod (R848) and 852A. Thus, FIG. 1 demonstrates that BNT411 is more potent on human PBMCs regarding IFNα induction than competitor compounds like resiquimod (R848). Since IFNa is a key driver of the mode of action provided by TLR7 agonists and an important biomarker for TLR7 activation, it can be expected that upon administration BNT411 will exert its effects as selective TLR7 agonist, in particular binding to TLR7 on plasmacytoid dendritic cells (pDCs), triggering their activation and maturation to antigen-presenting cells (APCs), the downstream activation and expansion of cytotoxic CD8⁺ T cells and the broad modulation of the innate immune system thereby enhancing pre-existing anti-tumor responses and inducing de novo responses, especially in combination with cytotoxic therapies and immune checkpoint inhibitors.

### Example 2

This example was done in order to determine a suitable starting dose of BNT411 for further in vivo experiments, in particular in tumor mouse models.

Female BALB/c mice were retro-orbitally administered with doses of 3, 5, 10, and 15 mg/kg BNT411. Blood samples were drawn 1 h after compound application and analyzed with mouse IFNa ELISA. The results of the IFNa ELISA are shown in FIG. 2.

As can be seen from FIG. 2, a dose of 3 mg/kg BNT411 induced a stronger IFNa secretion as compared to higher BNT411 doses, supporting a bell-shaped concentration-response-curve as observed in human in vitro assays with whole blood preparations. Thus, the dose of 3 mg/kg BNT411 was used for further in vivo experiments in mice.

### Example 3

This experiment has been performed in order to assess the anti-tumor efficacy of BNT411 alone. To this end, the CT26 tumor model has been utilized.

Eight doses of BNT411 were administered i.v. at a dose level of 3 mg/kg eight times with 4 to 5 days interval (on days 14, 19, 23, 28, 33, 37, 42, and 47); cf., FIG. 4A. At start of treatment, tumors had an average volume of 10 mm³. A control group (n=13) received vehicle alone. Adaptive immunity was evaluated by analyzing gp70-antigen-specific T cell populations in blood and splenocytes 3 and 5 weeks after the first study drug administration. Gp70 is the immune dominant CD8⁺ T-cell recognized antigen of the CT26 tumor model. The results of this experiment are shown in FIG. 4B.

As can be seen from FIG. 4B, BNT411 showed potent tumor growth inhibition with a test/control value of 43.3% on day 35 of the experiment. A Mann-Whitney test of individual tumor volumes on the same day showed a trend towards statistical significance (p=0.0513). On day 49, the last day of the experiment, 8 of 13 BNT411 treated animals and 2 of 13 vehicle treated animals were still alive.

Furthermore, BNT411 treatment resulted in a 50-fold increase in blood IFNα levels measured 1 h after the first dosing compared with the control group. Increased frequencies of gp70+ T-cells in blood were observed in BNT411-treated animals compared against the control group on day 35 (8.72% vs. 2.15%) and in splenocytes on day 50 (0.76% vs. 0.44%).

Blood parameters (alanine transaminase, aspartate transaminase, creatine kinase, glutamate dehydrogenase, lactate dehydrogenase, and bilirubin) measured on day 19 in serum were comparable for the BNT411 and control groups.

Thus, this example demonstrates that BNT411 exerts anti-tumoral efficacy in immunogenic mouse tumor models in monotherapy as exemplified with mouse colorectal cancer cell line CT-26. In this context, BNT411 stimulates the innate and the adaptive immune system as could be shown by induction of IFNa secretion and the increase in frequency of gp70+ T-cells which are specific for the gp70 antigen present on the surface of CT-26 tumor cells. Therefore, it can be asserted that the anti-tumoral activity of BNT411 is in line with its postulated mode-of-action.

### Example 4

This experiment has been performed in order to assess the anti-tumor efficacy of BNT411 in mono- and combination therapy with oxaliplatin in the CT26 tumor model.

Oxaliplatin was administered intraperitoneally (i.p.) (5 mg/kg, four times with 4 to 5 days between injections). BNT411 was administered i.v. (single-dose level of 3 mg/kg, eight times with 2 to 5 days between injections and at least 2 days after oxaliplatin therapy); cf., FIG. 5A. Anti-tumor efficacy was evaluated by group mean tumor volumes in test versus control groups (vehicle only) on study days 33 and 42, (at least 50% of test and control animals alive). IFNα levels in plasma were analyzed 1 h after first BNT411 administration and gp70+ T-cell populations in blood on study days 34 and 41. The results of this experiment are shown in FIGS. 5B and 5C.

As can be seen from FIG. 5B, BNT411/oxaliplatin combination therapy showed strongest anti-tumor efficacy with a T/C value of 34.3% compared to 50.7% for oxaliplatin in monotherapy and 65.8% for BNT411 in monotherapy.

IFNα serum levels were statistically significantly increased in all BNT411 treated groups compared to the control group; cf., FIG. 5C. IFNα levels in the BNT411 monotherapy group were statistically significantly higher than in the combination of BNT411 and oxaliplatin (two-way analysis of variance (ANOVA test).

Tumor-specific gp70+ T-cells were induced in all BNT411 treated groups between study day 34 and 41 (Mann-Whitney test).

In conclusion, BNT411 showed anti-tumor activity in monotherapy, and combining BNT411 with oxaliplatin had additive effects on tumor growth retardation.

### Example 5

This experiment has been performed in order to assess the anti-tumor efficacy of BNT411 in mono- and combination therapy with an anti-programmed cell death 1 ligand 1 (PD-L1) antibody (anti-PD-L1-mIgGle3) in the CT26 tumor model.

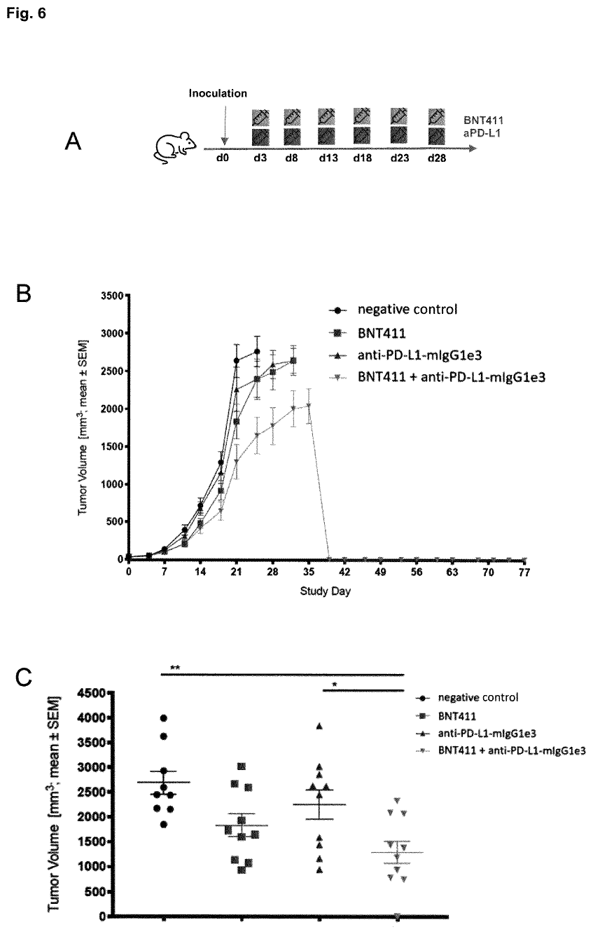

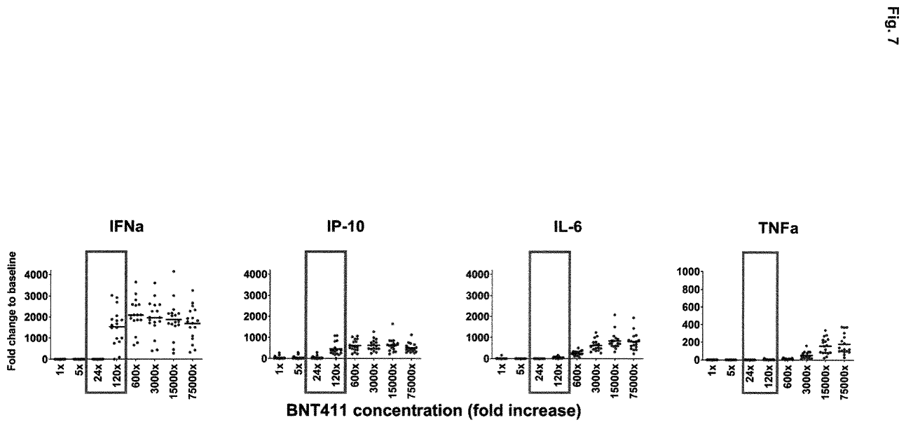

Four groups of 10 animals were inoculated with 1×10⁶ CT26 cells per animal into the dorsal right flank (day 0). The anti-PD-L1 antibody was administered intraperitoneally (i.p.; 200 μg per animal) and BNT411 was administered i.v. (3 mg/kg). All treatments were delivered once every five days for a total of six doses; cf., FIG. 6A. Body weight changes and tumor growth were assessed on study day 21 when there were at least nine animals alive in each group. One mouse treated with both anti-PD-L1 antibody and BNT411 cleared the initial tumor. This animal was re-inoculated with CT26 cells into the contralateral flank on study day 77 and received no further treatment. The results of this experiment are shown in FIGS. 6B and 6C.

As can be seen from FIGS. 6B and 6C, the combination of BNT411 with anti-PD-L1-mIgGle3 resulted in significant (p≤0.05) inhibition of tumor growth and a significantly (p≤0.05) smaller increase in body weight compared to the negative control treatment (isotype control antibody plus 0.9% saline) or anti-PD-L1-mIgGle3 alone after five doses of treatment. There was a significant (p≤0.05) survival benefit with all the test article treatments over the negative control treatment (isotype control antibody plus 0.9% saline), however, there were no significant differences between the test article treatments.

Tumor growth from the initial inoculation was successfully cleared in one animal in the group treated with the combination of BNT411 and anti-PD-L1-mIgGle3; cf., FIG. 6B. Secondary inoculation of this tumor-free animal with the same tumor entity induced initial CT26 tumor growth followed by rapid clearance of the CT26 tumor, although the animal remained untreated after re-inoculation.

In conclusion, BNT411 in combination with the murine PD-L1 antibody showed tumor growth retardation and survival benefit. Tumor clearance and subsequent immunity against tumor re-inoculation in one animal of the combination treatment group (BNT411 and anti-PD-L1-mIgGle3) suggests formation of an anti-tumor immune memory during treatment. Throughout treatment, both BNT411 and anti-PD-L1-mIgGle3 as well as the combination of both were well tolerated. There were no adverse clinical effects, higher toxicities, or body weight loss associated with the treatments.

### Example 6

This experiment has been performed in order to assess whether the beneficial effects seen in mice can be also achieved in humans.

Human whole blood was obtained from healthy donors and treated in vitro with BNT411 (concentration range: 0.1 nM to 10 μM). 24 h after stimulation secretion of several cytokines, including IFNα, IP-10, IL-6, and TNFα, was analyzed by a multiplexed CBA. The results of the CBA measurement are shown in FIG. 7.

As can be seen from FIG. 7, at very low doses (120 k=0.012 μM), BNT411 induced significant IFNα release. Importantly, proinflammatory cytokines like IL-6 and TNFa and regulatory cytokines like IL-10 were only induced at higher concentrations (well above the concentrations maximally inducing IFNα) starting at about 0.06 μM.

In conclusion, IFNα secretion was induced at 5- to 25-fold lower concentrations than needed for the induction of proinflammatory and regulatory cytokines such as IL-6, TNFα, and IL-10. Moreover, the BNT411 concentration for maximal induction of IFNα was 25- to 100-fold lower than the concentration necessary for maximal induction of IL-6, TNFα, and IL-10. This further confirms the sufficiently broad therapeutic window to separate the intended secretion of IFNα from that of proinflammatory cytokines.

### Example 7

This experiment has been performed in order to assess whether the beneficial effects seen in vivo in mice and in vitro in humans can be also achieved in human patients.

To this end, a trial was designed to assess the safety profile of BNT411 in monotherapy and in combination with atezolizumab, etoposide, and carboplatin (atezo/EC) and to determine the maximum tolerated dose/recommended phase 2 dose (MTD/RP2D). This trial comprised two parts (1A and 1B) with dose escalation, wherein the patients of part 1A received BNT411 monotherapy and patients of part 1B received BNT411 together with atezolizumab, etoposide, and carboplatin. The patients had solid tumor (preferably ES-SCLC for part 1B) and received the drugs according to the following dosing schedule (until disease progression, unacceptable toxicity, or death):
Part 1A: 3-week cycles, BNT411 i.v. Q1W for 4 cycles, then Q3W
Part 1B: 3-week cycles, BNT411 i.v. Q1W for 4 cycles, then Q3W; atezo/EC Q3W

The eight escalating dose levels (DL1 to DL8) for BNT411 are shown in Table 4:

TABLE 4
Escalating dose levels (DL1 to DL8) for BNT411

| | DL1 | DL2 | DL3 | DL4 | DL5 | DL6 | DL7 | DL8 |
|---|---|---|---|---|---|---|---|---|
| [μg/kg] | 0.05 | 0.15 | 0.45 | 1.2 | 2.4 | 4.8 | 9.6 | 16 |

DL1-DL3 of Part 1A were single-patient cohorts, all other dose levels were 3+3 cohorts.

Efficacy was assessed by CT/MRI imaging according to RECIST 1.1 and iRECIST every 6 weeks for 48 weeks, and every 12 weeks thereafter. Safety was assessed by physical examination, vital signs, electrocardiogram, clinical laboratory assessments, eye examinations, ECOG, pregnancy testing, and ECHO/MUGA. Biomarker assessments included tumor biopsies and blood sampling for cytokine analysis, immune phenotyping, tumor mutational burden, and TCR profiling.

As of Aug. 26, 2021, the patients were located in seven trial centers (UK, USA, Spain, Germany). Twenty-five patients were screened (Part 1A: 22; Part 1B: 3) with seven screen failures (all Part 1A). Eighteen patients enrolled (Part 1A: 15; Part 1B: 3), wherein fifteen patients were evaluable (Part 1A: 12; Part 1B: 3). Two Part 1A patients were replaced (at DL1 due to incorrect dosing and at DL5 due to PD before DLT evaluation completed). One Part 1A patient dropped out of trial before receiving treatment. The demographics and clinical characteristics of these patients are shown in Table 5. The Treatment emergent adverse events (TEAEs) related to BNT411 monotherapy and combination therapy are shown in Tables 6 and 7, respectively.

TABLE 5
Patient demographics and clinical characteristics

| Baseline characteristic | N = 18 |
|---|---|
| Male/Female | 10 (56%)/8 (44%) |
| Median age, years (range) | 58 (32-78) |
| Tumor type | |
| Cervical cancer | 1 |
| Colon cancer | 2 |
| Hepatic cancer | 1 |
| Malignant melanoma | 1 |
| Malignant solitary fibrous tumor | 1 |
| NSCLC | 1 |
| Ovarian cancer | 1 |
| Pancreatic carcinoma | 2 |
| Prostate cancer | 2 |
| Rectal cancer | 1 |
| SCLC | 4 |
| Ureteral cancer | 1 |
| Prior lines of systemic therapy, median (range) | 2 (0-5) |
| ≤1 | 6 (33%) |
| ≥2 | 12 (67%) |
| Prior anti-PD-1/PD-L1 therapy | 6 (33%) |

TABLE 6
TEAEs related to BNT411 monotherapy

| System organ classes/ | Grade | Grade | Grade | Grade |
|---|---|---|---|---|
| preferred terms | 1 | 2 | 3 | 4 |
| Blood and lymphatic | | | | |
| system disorders | | | | |
| Anaemia | 1 | | 1 | 2 (13%) |
| Neutropenia | 1 | | | 1 (7%) |
| Gastrointestinal disorders | | | | |
| Diarrhoea | | 1 | | 1 (7%) |
| Nausea | 1 | | | 1 (7%) |
| General disorders and | | | | |
| administration site conditions | | | | |
| Chills | 2 | | | 2 (13%) |
| Fatigue | | 1 | | 1 (7%) |
| Illness | 1 | | | 1 (7%) |
| Pyrexia | 1 | 2 | | 3 (20%) |
| Hepatobiliary disorders | | | | |
| Hyperbilirubinaemia | 1 | | | 1 (7%) |
| Injury, poisoning and | | | | |
| procedural complications | | | | |
| Infusion related reaction | | 1 | | 1 (7%) |
| Investigations | | | | |
| Blood bilirubin increased | 1 | | | 1 (7%) |
| Nervous system disorders | | | | |
| Headache | 1 | | | 1 (7%) |
| Skin and subcutaneous | | | | |
| tissue disorders | | | | |
| Pruritus | 1 | | | 1 (7%) |
| Vascular disorders | | | | |
| Hypotension | 1 | | | 1 (7%) |
| Total number of patients | | | | 7 (47%) |
| with TEAEs: | | | | |

TABLE 7
TEAEs related to BNT411 combination therapy with atezo/EC

| | Grade | Grade | Grade | Grade |
|---|---|---|---|---|
| System organ classes/preferred terms | 1 | 2 | 3 | 4 |
| General disorders and | | | | |
| administration site conditions | | | | |
| Pyrexia* | 1 | | | 1 (33%) |
| Infections and infestations | | | | |
| Pneumonia | | | 1 | 1 (33%) |
| Total number of patients with TEAEs: | | | | 2 (67%) |

*Assessed by investigator as unrelated to BNT411, serious due to prolonged hospitalization

As can be seen from Tables 6 and 7, BNT411 has an acceptable safety profile at all doses tested as of Aug. 26, 2021 (up to DL6). Most frequent adverse events (AEs) related to BNT411 monotherapy were pyrexia (n=3 patients [20%] grades 1 and 2, Part 1A dose levels (DLs) 1*, 2, and 6), chills (n=2 patients, [13%], grade 1, Part 1A DL1 and DL6) and anaemia (n=2 patients, [13%], grades 1 and 3, Part 1A DL4 and DL5). The affected patient (marked by *) was enrolled at DL1 but received a 22-fold higher dose (close to DL4). There was no dose limiting toxicity (DLT), or related grade 4-5 AEs in either Part 1A or Part 1B. Two serious adverse events with a possible causal relationship to BNT411 monotherapy were two cases of infusion-related reaction (grades 1 and 2, affecting the same patient in Part 1A DL6). One serious adverse event with a possible causal relationship to BNT411+atezo/EC was pneumonia (grade 3, Part 1B DL4). This event was resolved with antibiotic treatment, the cause of this event was considered multifactorial and could either be the underlying disease, or immune activation caused by either BNT411 or atezo/EC.

The results of the cytokine measurement of patients treated with BNT411 monotherapy are shown in FIG. 8, wherein the mean maximum fold change (+SD) calculated from the highest cytokine value observed on day 1 in cycle 1 relative to baseline is depicted. IFNa and IL-6 peaked 4 to 8 hours after treatment, IP-10 and TNFa peaked 2.5 to 8 hours after treatment. No meaningful cytokine changes were observed in DL1A, DL2A, or DL3A.

As can be seen from FIG. 8, there was a dose-dependent cytokine release for ten patients from Part 1A: DL4A (3 patients), DL5A (4 patients), and DL6A (3 patients). A substantial induction of IFNa, IP-10, and other mode-of-action relevant cytokines were observed at DL6A. Importantly, TNFa induction levels remained unchanged; some increase in IL-6 induction observed at DL6A. Thus, these data demonstrate that administration of BNT411 to human patients was well tolerated at all doses tested and results in a type-I interferon-dominated cytokine profile which is in line with the anticipated mode-of-action of BNT411 as selective TLR7 agonist.

In view of the above, it can be concluded that BNT411 has an acceptable safety profile at all doses up to DL6. Furthermore, at DL6A, BNT411 induces a substantial type-I interferon-dominated cytokine response, while levels of IL-6 and TNFa remain relatively low, in line with anticipated mode-of-action of BNT411 as selective TLR7 agonist. The pharmacodynamics responses resulting from the administration of BNT411 at the dose levels tested warrant further evaluation of BNT411 in various cancer indications, as monotherapy and in combination with atezo/EC and other immunotherapy-based regimens.

### Data Update for Part 1A—October 2022

The trial population consists of histologically confirmed solid tumor patients with metastatic or unresectable disease for which there is no available standard therapy likely to confer clinical benefit, or patients who are not candidates for such available therapy.

BNT411 appears to have an acceptable overall safety profile up to DL6.

As of the data cutoff date (Oct. 13, 2022), one patient started with BNT411 at DL4, the dose was increased to DL5 on C8D1 and further increased to DL6 on C12D1. The patient had stable disease (as per RECIST 1.1) until trial Day 449. A second patient is on BNT411 at an intermediate dose level between DL6 and DL7. On trial Day 86, there was a −26.2% change in sum of target lesion diameters compared with baseline. As of Day 420, the patient had stable disease (as per RECIST 1.1) and is still on treatment. A third patient is on BNT411 at DL7, the patient had a partial response (as per RECIST 1.1) on trial Day 251 and is still on the trial treatment with a partial response. These durable tumor responses following BNT411 treatment are encouraging early signs of possible efficacy of BNT411 in the treatment of various solid tumors.

In view of the above, it appears that BNT411 could potentially have an acceptable overall safety profile at therapeutically relevant dose levels. Promising efficacy signals were observed in patients across different tumor types. The tumor response and disease stability are supported by pharmacodynamic activity defined by induction of IFN-a and IP-10, which are increased following BNT411 administration at DL5 onwards and substantially observed starting at DL6. Furthermore, at DL6, BNT411 induces a substantial type-I IFN-dominated cytokine response, while acceptable levels of IL-6 and low levels of TNFa induction are observed, in line with the anticipated mode-of-action of BNT411 as a selective TLR7 agonist. The preliminary safety profile, efficacy signals and pharmacodynamic responses resulting from the administration of BNT411 warrant further investigation of BNT411 in various cancer indications.

1. A compound having the following formula 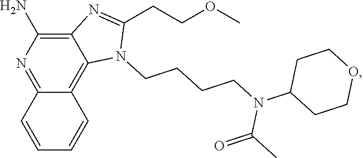 [SMILES: COCCc1nc2c(N)nc3ccccc3c2n1CCCCN(C(C)=O)C1CCOCC1]
or a pharmaceutically acceptable solvate or salt thereof, for use in a method for reducing or preventing progression of a tumor or treating cancer in a subject, said method comprising administering to said subject the compound in a suitable amount.

2. The compound for use of claim 1, wherein the suitable amount of the compound is a therapeutically effective and safe amount.

3. The compound for use of any one of the preceding claims, wherein the suitable amount of the compound is about 0.1 μg/kg to about 50 μg/kg body weight or about 8 μg to about 4000 μg in total; and/or about 0.23×10⁻⁹ mol/kg body weight to about 114×10⁻⁹ mol/kg body weight or about 18×10⁻⁹ mol to about 9100×10⁻⁹ mol in total.

4. The compound for use of any one of the preceding claims, wherein the suitable amount of the compound is about 0.5 μg/kg to about 20 μg/kg body weight or about 40 μg to about 1600 μg in total; and/or about 1.14×10⁻⁹ mol/kg body weight to about 45.5×10⁻⁹ mol/kg body weight or about 91×10⁻⁹ mol to about 3640×10⁻⁹ mol in total.

5. The compound for use of any one of the preceding claims, wherein the suitable amount of the compound is about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total.

6. The compound for use of any one of claims 1 to 5, wherein the suitable amount of the compound is about 2.4 μg/kg to about 9.6 μg/kg body weight or about 192 μg to about 768 μg in total; and/or about 5.46×10⁻⁹ mol/kg body weight to about 21.8×10⁻⁹ mol/kg body weight or about 437×10⁻⁹ mol to about 1747×10⁻⁹ mol in total.

7. The compound for use of any one of claims 1 to 5, wherein the suitable amount of the compound is about 4.8 μg/kg to about 9.6 μg/kg body weight or about 384 μg to about 768 μg in total; and/or about 10.9×10⁻⁹ mol/kg body weight to about 21.8×10⁻⁹ mol/kg body weight or about 873×10⁻⁹ mol to about 1747×10⁻⁹ mol in total.

8. The compound for use of any one of claims 1 to 5, wherein the suitable amount of the compound is about 4.8 μg/kg body weight or about 384 μg in total; and/or about 10.9×10⁻⁹ mol/kg body weight or about 874×10⁻⁹ mol in total.

9. The compound for use of any one of the preceding claims, wherein the compound is a solvate.

10. The compound for use of claim 9, wherein the solvate is selected from the group consisting of a hydrate and a dimethylsulfoxide solvate.

11. The compound for use of any one of claims 1 to 8, wherein the compound is anhydrous.

12. The compound for use of any one of the preceding claims, wherein the compound is administered systemically, preferably intravenously.

13. The compound for use of any one of the preceding claims, wherein the subject is a human subject.

14. The compound for use of any one of the preceding claims, wherein the tumor or cancer is a solid tumor or cancer.

15. The compound for use of any one of the preceding claims, wherein the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

16. The compound for use of any one of the preceding claims, wherein the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

17. The compound for use of any one of the preceding claims, wherein the tumor or cancer is a small cell lung cancer (SCLC), such as extensive-stage small cell lung cancer (ES-SCLC).

18. The compound for use of any one of the preceding claims, wherein the subject is chemotherapy-naïve.

19. The compound for use of any one of the preceding claims, wherein the compound is administered in at least one treatment cycle, each treatment cycle being three weeks (21 days).

20. The compound for use of any one of the preceding claims, wherein one dose of the compound is administered once a week (Q1W) for at least 1 treatment cycle.

21. The compound for use of any one of the preceding claims, wherein one dose of the compound is administered Q1W for at least 4 treatment cycles and then every 3 weeks (Q3W).

22. The compound for use of any one of the preceding claims, wherein each dose of the compound is infused over a minimum of 10 minutes, such as over a minimum of 15 minutes, a minimum of 20 minutes, a minimum of 25 minutes or a minimum of 30 minutes.

23. The compound for use of any one of the preceding claims, wherein the method further comprises administering to said subject one or more additional therapeutic agents.

24. The compound for use of claim 23, wherein the one or more additional therapeutic agents are selected from the group consisting of checkpoint inhibitors (CPIs), platinum-based chemotherapeutic agents, topoisomerase inhibitors, and combinations thereof.

25. The compound for use of claim 23 or 24, wherein the one or more additional therapeutic agents are selected from the group consisting of atezolizumab, carboplatin, etoposide, and combinations thereof.

26. The compound for use of any one of claims 23 to 25, wherein the one or more additional therapeutic agents are a combination of atezolizumab, carboplatin, and etoposide.

27. The compound for use of any one of claims 23 to 26, wherein the one or more additional therapeutic agents are administered in at least one treatment cycle, each treatment cycle being three weeks (21 days).

28. The compound for use of any one of claims 23 to 27, wherein one dose of each of the one or more additional therapeutic agents is administered at least Q3W for at least 1 treatment cycle, each treatment cycle being three weeks (21 days).

29. The compound for use of any one of claims 23 to 28, wherein the one or more additional therapeutic agents are a combination of atezolizumab, carboplatin, and etoposide; one dose of atezolizumab is administered Q3W (preferably at day 1 of a treatment cycle); one dose of carboplatin is administered Q3W (preferably at day 1 of a treatment cycle) for 4 treatment cycles; and one dose of etoposide is administered three times in the first week of a treatment cycle (preferably at day 1, day 2 and day 3 of a treatment cycle) for 4 treatment cycles, each treatment cycle being three weeks (21 days).

30. The compound for use of claim 29, wherein one dose of the compound is administered Q1W (preferably at day 2, day 8, and day 15 of a treatment cycle) for 4 treatment cycles and then Q3W (preferably at day 2 of treatment cycle 5 and each subsequent treatment cycle).

31. A composition comprising a compound having the following formula 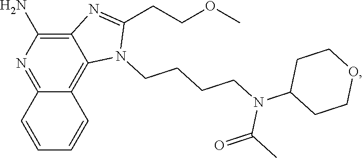 [SMILES: COCCc1nc2c(N)nc3ccccc3c2n1CCCCN(C(C)=O)C1CCOCC1]
or a pharmaceutically acceptable solvate or salt thereof, wherein the amount of the compound in the composition is between about 8 μg to 4000 μg or about 18×10⁻⁹ mol to about 9100×10⁻⁹ mol.

32. The composition of claim 31, comprising from about 40 μg to about 1600 μg or about 91×10⁻⁹ mol to about 3640×10⁻⁹ mol of said compound, preferably from about 160 μg to about 800 μg or about 364×10⁻⁹ mol to about 1820×10⁻⁹ mol of said compound.

33. The composition of claim 31 or 32, comprising from about 192 μg to about 768 μg or about 437×10⁻⁹ mol to about 1747×10⁻⁹ mol of said compound.

34. The composition of claim 31 or 32, comprising from about 384 μg to about 768 μg or about 873×10⁻⁹ mol to about 1747×10⁻⁹ mol of said compound.

35. The composition of claim 31 or 32, comprising about 384 μg or about 874×10⁻⁹ mol of said compound.

36. The composition of any one of claims 31 to 35, wherein the composition is for systemic administration.

37. The composition of any one of claims 31 to 36, wherein the composition is for injection or infusion, such as intravenous injection or infusion.

38. The composition of any one of claims 31 to 37, wherein the compound is in aqueous solution, such as in 0.9% NaCl (saline), at a volume of 1-500 ml, such as 5-250 ml.

39. The composition of any one of claims 31 to 38, said composition being a dosage unit form.

40. The composition of any one of claims 31 to 39 for use in a method for reducing or preventing progression of a tumor or treating cancer in a subject.

41. The composition for use of claim 40, wherein the composition is administered to the subject in an amount so as to provide about 0.1 μg/kg to about 50 μg/kg body weight of the compound or about 8 μg to about 4000 μg of the compound in total; and/or about 0.23×10⁻⁹ mol/kg body weight to about 114×10⁻⁹ mol/kg body weight of the compound or about 18×10⁻⁹ mol to about 9100×10⁻⁹ mol of the compound in total.

42. The compound for use of claim 40 or 41, wherein the composition is administered to the subject in an amount so as to provide about 0.5 μg/kg to about 20 μg/kg body weight of the compound or about 40 μg to about 1600 μg of the compound in total; and/or about 1.14×10⁻⁹ mol/kg body weight to about 45.5×10⁻⁹ mol/kg body weight of the compound or about 91×10⁻⁹ mol to about 3640×10⁻⁹ mol of the compound in total.

43. The compound for use of any one of claims 40 to 42, wherein the composition is administered to the subject in an amount so as to provide about 2 μg/kg to about 10 μg/kg body weight of the compound or about 160 μg to about 800 μg of the compound in total; and/or about 4.55×10⁻⁹ mol/kg body weight to about 22.8×10⁻⁹ mol/kg body weight of the compound or about 364×10⁻⁹ mol to about 1820×10⁻⁹ mol of the compound in total.

44. The compound for use of any one of claims 40 to 43, wherein the composition is administered to the subject in an amount so as to provide about 2.4 μg/kg to about 9.6 μg/kg body weight of the compound or about 192 μg to about 768 μg of the compound in total; and/or about 5.46×10⁻⁹ mol/kg body weight to about 21.8×10⁻⁹ mol/kg body weight of the compound or about 437×10⁻⁹ mol to about 1747×10⁻⁹ mol of the compound in total.

45. The compound for use of any one of claims 40 to 43, wherein the composition is administered to the subject in an amount so as to provide about 4.8 μg/kg to about 9.6 μg/kg body weight of the compound or about 384 μg to about 768 μg of the compound in total; and/or about 10.9×10⁻⁹ mol/kg body weight to about 21.8×10⁻⁹ mol/kg body weight of the compound or about 873×10⁻⁹ mol to about 1747×10⁻⁹ mol of the compound in total.

46. The compound for use of any one of claims 40 to 43, wherein the composition is administered to the subject in an amount so as to provide about 4.8 μg/kg body weight of the compound or about 384 μg of the compound in total; and/or about 10.9×10⁻⁹ mol/kg body weight of the compound or about 874×10⁻⁹ mol of the compound in total.

47. The composition for use of any one of claims 40 to 46, wherein the subject is a human subject.

48. The composition for use of any one of claims 40 to 47, wherein the tumor or cancer is a solid tumor or cancer.

49. The composition for use of any one of claims 40 to 48, wherein the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

50. The composition for use of any one of claims 40 to 49, wherein the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

51. The composition for use of any one of claims 40 to 50, wherein the tumor or cancer is a small cell lung cancer (SCLC), such as extensive-stage small cell lung cancer (ES-SCLC).

52. The composition for use of any one of claims 40 to 51, wherein the subject is chemotherapy-naïve.

53. The composition for use of any one of claims 40 to 52, wherein the composition is administered in at least one treatment cycle, each treatment cycle being three weeks (21 days).

54. The composition for use of any one of claims 40 to 53, wherein one dose of the composition is administered once a week (Q1W) for at least 1 treatment cycle.

55. The composition for use of any one of claims 40 to 54, wherein one dose of the composition is administered Q1W for at least 4 treatment cycles and then every 3 weeks (Q3W).

56. The composition for use of any one of claims 40 to 55, wherein the composition is infused over a minimum of 10 minutes, such as over a minimum of 15 minutes, a minimum of 20 minutes, a minimum of 25 minutes or a minimum of 30 minutes.

57. The composition for use of any one of claims 40 to 56, wherein the method further comprises administering to said subject one or more additional therapeutic agents.

58. The composition for use of claim 57, wherein the one or more additional therapeutic agents are selected from the group consisting of checkpoint inhibitors (CPIs), platinum-based chemotherapeutic agents, topoisomerase inhibitors, and combinations thereof.

59. The composition for use of claim 57 or 58, wherein the one or more additional therapeutic agents are selected from the group consisting of atezolizumab, carboplatin, etoposide, and combinations thereof.

60. The composition for use of any one of claims 57 to 59, wherein the one or more additional therapeutic agents are a combination of atezolizumab, carboplatin, and etoposide.

61. The composition for use of any one of claims 57 to 60, wherein the one or more additional therapeutic agents are administered in at least one treatment cycle, each treatment cycle being three weeks (21 days).

62. The composition for use of any one of claims 57 to 61, wherein one dose of each of the one or more additional therapeutic agents is administered at least Q3W for at least 1 treatment cycle, each treatment cycle being three weeks (21 days).

63. The composition for use of any one of claims 57 to 62, wherein the one or more additional therapeutic agents are a combination of atezolizumab, carboplatin, and etoposide; one dose of atezolizumab is administered Q3W (preferably at day 1 of a treatment cycle); one dose of carboplatin is administered Q3W (preferably at day 1 of a treatment cycle) for 4 treatment cycles; and one dose of etoposide is administered three times in the first week of a treatment cycle (preferably at day 1, day 2 and day 3 of a treatment cycle) for 4 treatment cycles, each treatment cycle being three weeks (21 days).

64. The composition for use of claim 63, wherein one dose of the composition is administered Q1W (preferably at day 2, day 8, and day 15 of a treatment cycle) for 4 treatment cycles and then Q3W (preferably at day 2 of treatment cycle 5 and each subsequent treatment cycle).

65. A kit comprising (i) a compound having the following formula 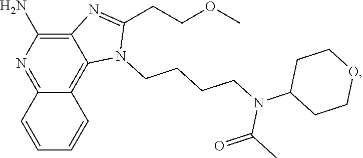 [SMILES: COCCc1nc2c(N)nc3ccccc3c2n1CCCCN(C(C)=O)C1CCOCC1]
or a pharmaceutically acceptable solvate or salt thereof, and (ii) one or more additional therapeutic agents selected from the group consisting of checkpoint inhibitors (CPIs), platinum-based chemotherapeutic agents, topoisomerase inhibitors, and combinations thereof.

66. The kit of claim 65, wherein the one or more additional therapeutic agents are selected from the group consisting of atezolizumab, carboplatin, etoposide, and combinations thereof.

67. The kit of claim 65 or 66, wherein the compound and the one or more additional therapeutic agents are for systemic administration, in particular for injection or infusion, such as intravenous injection or infusion.

68. The kit of any one of claims 65 to 67 for use in a method for reducing or preventing progression of a tumor or treating cancer in a subject.

69. The kit for use of claim 68, wherein the compound is administered to the subject in an amount of about 0.1 μg/kg to about 50 μg/kg body weight or about 8 μg to about 4000 μg in total; and/or about 0.23×10⁻⁹ mol/kg body weight to about 114×10⁻⁹ mol/kg body weight or about 18×10⁻⁹ mol to about 9100×10⁻⁹ mol in total.

70. The kit for use of claim 68 or 69, wherein the compound is administered to the subject in an amount of about 0.5 μg/kg to about 20 μg/kg body weight or about 40 μg to about 1600 μg in total; and/or about 1.14×10⁻⁹ mol/kg body weight to about 45.5×10⁻⁹ mol/kg body weight or about 91×10⁻⁹ mol to about 3640×10⁻⁹ mol in total.

71. The kit for use of any one of claims 68 to 70, wherein the compound is administered to the subject in an amount of about 2 μg/kg to about 10 μg/kg body weight or about 160 μg to about 800 μg in total; and/or about 4.55×10⁻⁹ mol/kg body weight to about 22.8×10⁻⁹ mol/kg body weight or about 364×10⁻⁹ mol to about 1820×10⁻⁹ mol in total.

72. The kit for use of any one of claims 68 to 71, wherein the compound is administered to the subject in an amount of about 2.4 μg/kg to about 9.6 μg/kg body weight or about 192 μg to about 768 μg in total; and/or about 5.46×10⁻⁹ mol/kg body weight to about 21.8×10⁻⁹ mol/kg body weight or about 437×10⁻⁹ mol to about 1747×10⁻⁹ mol in total.

73. The kit for use of any one of claims 68 to 71, wherein the compound is administered to the subject in an amount of about 4.8 μg/kg to about 9.6 μg/kg body weight or about 384 μg to about 768 μg in total; and/or about 10.9×10⁻⁹ mol/kg body weight to about 21.8×10⁻⁹ mol/kg body weight or about 873×10⁻⁹ mol to about 1747×10⁻⁹ mol in total.

74. The kit for use of any one of claims 68 to 71, wherein the compound is administered to the subject in an amount of about 4.8 μg/kg body weight or about 384 μg in total; and/or about 10.9×10⁻⁹ mol/kg body weight or about 874×10⁻⁹ mol in total.

75. The kit for use of any one of claims 68 to 74, wherein the subject is a human subject.

76. The kit for use of any one of claims 68 to 75, wherein the tumor or cancer is a solid tumor or cancer.

77. The kit for use of any one of claims 68 to 76, wherein the tumor or cancer is selected from the group consisting of melanoma, ovarian cancer, lung cancer (e.g., non-small cell lung cancer (NSCLC) or small cell lung cancer (SCLC)), colon cancer, rectal cancer, colorectal cancer, head and neck cancer, gastric cancer, breast cancer, ovarian cancer, renal cancer, urothelial cancer, bladder cancer, esophageal cancer, prostate cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, ureteral cancer, penile cancer, cervical cancer, Hodgkin's lymphoma, non-Hodgkin's lymphoma, Merkel cell carcinoma, malignant solitary fibrous tumor, and mesothelioma.

78. The kit for use of any one of claims 68 to 77, wherein the tumor or cancer is selected from the group consisting of lung cancer (e.g., non-small cell lung cancer (NSCLC) or small cell lung cancer (SCLC)), melanoma, colon cancer, rectal cancer, cervical cancer, ovarian cancer, hepatic cancer, malignant solitary fibrous tumor, prostate cancer, ureteral cancer, and pancreatic cancer.

79. The kit for use of any one of claims 68 to 78, wherein the tumor or cancer is a small cell lung cancer (SCLC), such as extensive-stage small cell lung cancer (ES-SCLC).

80. The kit for use of any one of claims 68 to 79, wherein the subject is chemotherapy-naïve.

81. The kit for use of any one of claims 68 to 80, wherein the compound is administered in at least one treatment cycle, each treatment cycle being three weeks (21 days).

82. The kit for use of any one of claims 68 to 81, wherein one dose of the compound is administered once a week (Q1W) for at least 1 treatment cycle.

83. The kit for use of any one of claims 68 to 82, wherein one dose of the compound is administered Q1W for at least 4 treatment cycles and then every 3 weeks (Q3W).

84. The kit for use of any one of claims 68 to 83, wherein each dose of the compound is infused over a minimum of 10 minutes, such as over a minimum of 15 minutes, a minimum of 20 minutes, a minimum of 25 minutes or a minimum of 30 minutes.

85. The kit for use of any one of claims 68 to 84, wherein the one or more additional therapeutic agents are administered in at least one treatment cycle, each treatment cycle being three weeks (21 days).

86. The kit for use of any one of claims 68 to 85, wherein one dose of each of the one or more additional therapeutic agents is administered at least Q3W for at least 1 treatment cycle, each treatment cycle being three weeks (21 days).

87. The kit for use of any one of claims 68 to 86, wherein the one or more additional therapeutic agents are a combination of atezolizumab, carboplatin, and etoposide; one dose of atezolizumab is administered Q3W (preferably at day 1 of a treatment cycle); one dose of carboplatin is administered Q3W (preferably at day 1 of a treatment cycle) for 4 treatment cycles; and one dose of etoposide is administered three times in the first week of a treatment cycle (preferably at day 1, day 2 and day 3 of a treatment cycle) for 4 treatment cycles, each treatment cycle being three weeks (21 days).

88. The kit for use of claim 87, wherein one dose of the compound is administered Q1W (preferably at day 2, day 8, and day 15 of a treatment cycle) for 4 treatment cycles and then Q3W (preferably at day 2 of treatment cycle 5 and each subsequent treatment cycle).
